(19) United States
(12) Patent Application Publication
(10) Pub. No.: US 2009/0192203 A1
(43) Pub. Date: Jul. 30, 2009

(54) COMPOUNDS AND COMPOSITIONS AS PPAR MODULATORS

(75) Inventors: Robert Epple, San Diego, CA (US); Christopher Cow, San Diego, CA (US); Mihai Azimioara, La Jolla, CA (US); Ross Russo, Encinitas, CA (US); Yongping Xie, San Diego, CA (US); Xing Wang, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 12/092,962

(22) PCT Filed: Nov. 7, 2006
(86) PCT No.: PCT/US2006/043342

§ 371 Date: May 7, 2008

Related U.S. Application Data
(60) Provisional application No. 60/734,683, filed on Nov. 7, 2005.

(51) Int. Cl. A61K 31/426; C07D 263/32; C07D 277/30; A61K 31/421; A61P 25/28

(52) U.S. Cl. 514/365; 548/236; 548/204; 514/372

(57) ABSTRACT

The invention provides compounds, pharmaceutical compositions comprising such compounds and methods of using such compounds to treat or prevent diseases or disorders associated with the activity of the Peroxisome Proliferator-Activated Receptor (PPAR) families.

### CROSS-REFERENCE TO RELATED APPLICATIONS

This application claims the benefit of priority to U.S. Provisional Patent Application No. 60/734,683, filed 7 Nov. 2005. The full disclosure of this application is incorporated herein by reference in its entirety and for all purposes.

### BACKGROUND OF THE INVENTION

1. Field of the Invention

The invention provides compounds, pharmaceutical compositions comprising such compounds and methods of using such compounds to treat or prevent diseases or disorders associated with the activity of the Peroxisome Proliferator-Activated Receptor (PPAR) families.

2. Background

Peroxisome Proliferator Activated Receptors (PPARs) are members of the nuclear hormone receptor super family, which are ligand-activated transcription factors regulating gene expression. Certain PPARs are associated with a number of disease states including dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, IBDs (irritable bowel disease), ulcerative colitis and Crohn's disease. Accordingly, molecules that modulate the activity of PPARs are useful as therapeutic agents in the treatment of such diseases.

### SUMMARY OF THE INVENTION

In one aspect, the present invention provides compounds of Formula I:

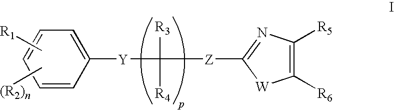

in which

n is selected from 0, 1, 2 and 3;

p is selected from 0, 1, 2 and 3;

Y is selected from O, S, NR₇ₐ and CR₇ₐR_{7b}; wherein R₇ₐ and R_{7b} are independently selected from hydrogen and C₁₋₆alkyl;

Z is selected from CR₈ₐR_{8b} and S; wherein R₈ₐ and R_{8b} are independently selected from hydrogen and C₁₋₆alkyl;

W is selected from O and S;

R₁ is selected from —X₁CR₉R₁₀X₂CO₂R₁₁, —X₁SCR₉R₁₀X₂CO₂R₁₁ and —X₁OCR₉R₁₀X₂CO₂R₁₁; wherein X₁ and X₂ are independently selected from a bond and C₁₋₄alkylene; and R₉ and R₁₀ are independently selected from hydrogen, C₁₋₄alkyl and C₁₋₄alkoxy; or R₉ and R₁₀ together with the carbon atom to which R₉ and R₁₀ are attached form C₃₋₁₂cycloalkyl; and R₁₁ is selected from hydrogen and C₁₋₆alkyl; each

R₂ is independently selected from halo, C₁₋₆alkyl, C₂₋₆alkenyl, C₁₋₄alkoxy, C₁₋₄alkylthio, C₃₋₁₂cycloalkyl, C₃₋₈heterocycloalkyl, C₆₋₁₀aryl and C₅₋₁₀heteroaryl; wherein any aryl, heteroaryl, cycloalkyl or heterocycloalkyl of R₂ is optionally substituted with 1 to 3 radicals independently selected from halo, C₁₋₆alkyl, C₁₋₆alkoxy, C₂₋₆alkenyl, C₁₋₆alkylthio, halo-substituted-C₁₋₆alkyl, halo-substituted-C₁₋₆alkoxy, —C(O)R₁₄ₐ and NR₁₄ₐR_{14b}; wherein R₁₄ₐ and R_{14b} are independently selected from hydrogen and C₁₋₆alkyl;

R₃ and R₄ are independently selected from hydrogen and C₁₋₆alkyl;

R₅ and R₆ are independently selected from hydrogen, C₁₋₆alkyl, C₃₋₁₂cycloalkyl, C₃₋₈heterocycloalkyl, C₆₋₁₀aryl and C₅₋₁₃heteroaryl;

wherein any aryl, heteroaryl, cycloalkyl and heterocycloalkyl of R₅ and R₆ is optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈heterocycloalkyl, C₆₋₁₀aryl, C₅₋₁₃heteroaryl, —XS(O)₀₋₂R¹², —XS(O)₀₋₂XR¹³, —XNR¹²R¹², —XNR¹²S(O)₀₋₂R¹², —XNR¹²C(O)R¹², —XC(O)NR¹²R¹², —XNR¹²C(O)R¹³, —XC(O)NR¹²R¹³, —XC(O)R¹³, —XNR¹²XR¹³ and —XOXR¹³; wherein any aryl, heteroaryl, cycloalkyl or heterocycloalkyl substituent is further optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl and halo-substituted-C₁₋₆alkoxy; wherein R¹³ is a bond or C₁₋₄alkylene; R¹² is selected from hydrogen and C₁₋₆alkyl; and R¹³ is selected from C₃₋₁₂cycloalkyl, C₃₋₈heterocycloalkyl, C₆₋₁₀aryl and C₅₋₁₀heteroaryl; wherein any aryl, heteroaryl, cycloalkyl or heterocycloalkyl of R¹³ is optionally substituted with 1 to 3 radicals independently selected from halo, nitro, cyano, C₁₋₆alkyl, C₁₋₆alkoxy, halo-substituted-C₁₋₆alkyl and halo-substituted-C₁₋₆alkoxy; with the proviso that either R₅ or R₆, but not both R₅ and R₆, must be hydrogen or methyl; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition that contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which modulation of PPAR activity can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which PPAR activity contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be either straight-chained or branched. C₁₋₆alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl can be phenyl or naphthyl, preferably phenyl. “Arylene” means a divalent radical derived from an aryl group. “Heteroaryl” is as defined for aryl where one or more of the ring members are a heteroatom. For example heteroaryl includes pyridyl, indolyl, indazolyl, quinoxalinyl, quinolinyl, benzofuranyl, benzopyranyl, benzothiopyranyl, benzo[1,3]dioxole, imidazolyl, benzoimidazolyl, pyrimidinyl, furanyl, oxazolyl, isoxazolyl, triazolyl, tetrazolyl, pyrazolyl, thienyl, etc. “C₆₋₁₀arylC₀₋₄alkyl” means an aryl as described above connected via a alkylene grouping. For example, C₆₋₁₀arylC₀₋₄alkyl includes phenethyl, benzyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc. “Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, etc.

“Halogen” (or halo) preferably represents chloro or fluoro, but can also be bromo or iodo.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of diseases in which modulation of PPAR activity can prevent, inhibit or ameliorate the pathology and/or symptomology of the diseases, which method comprises administering to the animal a therapeutically effective amount of a compound of Formula I. In one embodiment, with reference to compounds of Formula I, n is selected from 0, 1, 2 and 3;

p is selected from 0, 1 and 2;

Y is selected from O and S;

Z is selected from CR₈ₐR_{9b} and S; wherein R₈ₐ and R_{8b} are independently selected from hydrogen and C₁₋₆alkyl;

W is selected from O and S;

R₁ is selected from —X₁CR₉R₁₀X₂CO₂R₁₁, —X₁SCR₉R₁₀X₂CO₂R₁₁ and —X₁OCR₉R₁₀X₂CO₂R₁₁; wherein X₁ and X₂ are independently selected from a bond and C₁₋₄alkylene; and R₉ and R₁₀ are independently selected from hydrogen, C₁₋₄alkyl and C₁₋₄alkoxy; or R₉ and R₁₀ together with the carbon atom to which R₉ and R₁₀ are attached form C₃₋₁₂cycloalkyl; and R₁₁ is selected from hydrogen and C₁₋₆alkyl; each

R₂ is independently selected from C₁₋₆alkyl, C₂₋₆alkenyl, C₁₋₄alkoxy, C₁₋₄alkylthio, C₃₋₁₂cycloalkyl, C₃₋₈heterocycloalkyl, C₆₋₁₀aryl and C₅₋₁₀heteroaryl; wherein any aryl, heteroaryl, cycloalkyl or heterocycloalkyl of R₂ is optionally substituted with 1 to 3 radicals independently selected from halo, C₁₋₆alkoxy, C₁₋₆alkylthio, halo-substituted-C₁₋₆alkoxy, —C(O)R₁₄ₐ and NR₁₄ₐR_{14b}; wherein R₁₄ₐ and R_{14b} are independently selected from hydrogen and C₁₋₆alkyl;

R₃ and R⁴ are independently selected from hydrogen and C₁₋₆alkyl;

R₅ is C₆₋₁₀aryl optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈heterocycloalkyl, C₆₋₁₀aryl, C₅₋₁₃heteroaryl and —XNR¹²R¹²; wherein R¹² is selected from hydrogen and C₁₋₆alkyl; and

R₆ is selected from hydrogen and methyl.

In another embodiment, R₁ is selected from CH₂CR₅R₆CO₂H, —OCR₅R₆CO₂H, —SCR₅R₆CO₂H, —CR₅R₆CH₂CO₂H and —CR₅R₆CO₂H; wherein R₅ and R₆ are independently selected from hydrogen, methyl, methoxy and ethoxy; or R₅ and R₆ together with the carbon atom to which R₅ and R₆ are attached form cyclopentyl.

In another embodiment, each R₂ is independently selected from methyl, ethyl, cyclopropyl, methoxy, furanyl, phenyl, pyridinyl, thienyl, pyrrolidinyl and benzo[1,3]dioxolyl; wherein said pyridinyl or phenyl of R₂ is optionally substituted with 1 to 3 radicals independently selected from halo, methyl-carbonyl, dimethyl-amino, methoxy, halo-substituted-methoxy, methyl-thio, ethenyl, hexenyl and propyloxy.

Preferred compounds of Formula I are selected from: (5-Cyclopropyl-2-methyl-4-{3-[4-(4-trifluoromethyl-phenyl)-thiazol-2-yl]-propoxy}-phenoxy)-acetic acid; (3-{3-[4-(4-Trifluoromethyl-phenyl)-thiazol-2-yl]-propoxy}-phenyl)-acetic acid; (2-Methyl-4-{3-[4-(4-trifluoromethyl-phenyl)-thiazol-2-yl]-propoxy}-phenoxy)-acetic acid; 2-Methyl-2-(4-{3-[4-(4-trifluoromethyl-phenyl)-thiazol-2-yl]-propoxy}-phenoxy)-propionic acid; (2-Cyclopropyl-3-{3-[4-(4-trifluoromethyl-phenyl)-thiazol-2-yl]-propoxy}-phenyl)-acetic acid; (4-Cyclopropyl-3-{3-[4-(4-trifluoromethyl-phenyl)-thiazol-2-yl]-propoxy}-phenyl)-acetic acid; (±)-2-Ethoxy-3-(4-{3-[4-(4-trifluoromethyl-phenyl)-thiazol-2-yl]-propoxy}-phenyl)propionic acid; (±)-2-Methoxy-3-(4-{3-[4-(4-trifluoromethyl-phenyl)-thiazol-2-yl]-propoxy}-phenyl)-propionic acid; (3-{3-[4-(4-Trifluoromethyl-phenyl)-oxazol-2-yl]-propoxy}-phenyl)-acetic acid; (2-Methyl-4-{3-[4-(4-trifluoromethyl-phenyl)-oxazol-2-yl]-propoxy}-phenoxy)-acetic acid; (5-Cyclopropyl-2-methyl-4-{3-[4-(4-trifluoromethylphenyl)-oxazol-2-yl]-propoxy}-phenoxy)-acetic acid; 2-Methyl-2-(4-{3-[4-(4-trifluoromethyl-phenyl)-oxazol-2-yl]-propoxy}-phenoxy)-propionic acid; (2-Cyclopropyl-3-{3-[4-(4-trifluoromethyl-phenyl)-oxazol-2-yl]-propoxy}-phenyl)-acetic acid; (4-Cyclopropyl-3-{3-[4-(4-trifluoromethyl-phenyl)-oxazol-2-yl]-propoxy}-phenyl)-acetic acid; (3-{2-[4-(4-Trifluoromethyl-phenyl)-oxazol-2-yl]-ethoxy}-phenyl)-acetic acid; (3-{2-[4-(4-Trifluoromethoxy-phenyl)-oxazol-2-yl]-ethoxy}-phenyl)-acetic acid; (2-Cyclopropyl-3-{2-[4-(4-trifluoromethoxy-phenyl)-oxazol-2-yl]-ethoxy}-phenyl)-acetic acid; (4-Cyclopropyl-3-{2-[4-(4-trifluoromethoxy-phenyl)-oxazol-2-yl]-ethoxy}-phenyl)-acetic acid; (2-Cyclopropyl-3-{2-methyl-2-[4-(4-trifluoromethyl-phenyl)-oxazol-2-yl]-propoxy}-phenyl)-acetic acetic acid; (4-Cyclopropyl-3-{2-methyl-2-[4-(4-trifluoromethyl-phenyl)-oxazol-2-yl]-propoxy}-phenyl)-acetic acid; 3-(2-Methyl-4-{3-[4-(4-trifluoromethyl-phenyl)-thiazol-2-yl]-propoxy}-phenyl)-propionic acid; 3-(2-Cyclopropyl-5-{3-[4-(4-trifluoromethyl-phenyl)thiazol-2-yl]-propoxy}-phenyl)-propionic acid; 3-(5-Cyclopropyl-2-methyl-4-{3-[4-(4-trifluoromethyl-phenyl)-thiazol-2-yl]-propoxy}-phenyl)-propionic acid; 3-(4-Cyclopropyl-3-{3-[4-(4-trifluoromethyl-phenyl)-thiazol-2-yl]-propoxy}-phenyl)-propionic acid; 2-Methyl-2-(3-methyl-4-{3-[4-(4-trifluoromethyl-phenyl)-thiazol-2-yl]-propoxy}-phenoxy)-propionic acid; (±)-2-Methyl-3-(4-{3-[4-(4-trifluoromethyl-phenyl)-thiazol-2-yl]-propoxy}-phenyl)propionic acid; (±)-3-(4-{3-[4-(4-Trifluoromethyl-phenyl)-thiazol-2-yl]-propoxy}-phenyl)butyric acid; 2-Methyl-2-(2-methyl-4-{3-[4-(4-trifluoromethyl-phenyl)-thiazol-2-yl]-propoxy}-phenoxy)-propionic acid; 2-(2,3-Dimethyl-4-{3-[4-(4-trifluoromethyl-phenyl)thiazol-2-yl]-propoxy}-phenoxy)-2-methyl-propionic acid; (±)-2-Ethoxy-3-(2-methyl-4-{3-[4-(4-trifluoromethyl-phenyl)-thiazol-2-yl]-propoxy}-phenyl)-propionic acid; 2-(2,5-Dimethyl-4-{3-[4-(4-trifluoromethyl-phenyl)-thiazol-2-yl]-propylsulfanyl}-phenoxy)-2-methyl-propionic acid; (2,5-Dimethyl-4-{3-[4-(4-trifluoromethyl-phenyl)-thiazol-2-yl]-propoxy}-phenoxy)-acetic acid; (2,5-Dimethyl-4-{3-[4-(4-trifluoromethyl-phenyl)-thiazol-2-yl]-propylsulfanyl}-phenoxy)-acetic acid; 2-(2,5-Dimethyl-4-{3-[4-(4-trifluoromethylphenyl)-thiazol-2-yl]-propoxy}-phenoxy)-2-methyl-propionic acid; (3-Methoxy-4-{3-[4-(4-trifluoromethyl-phenyl)-thiazol-2-yl]-propoxy}-phenyl)-acetic acid; (4-Methoxy-3-{3-[4-(4-trifluoromethyl-phenyl)-thiazol-2-yl]-propoxy}-phenyl)-acetic acid; (±)-3-(2,5-Dimethyl-4-{3-[4-(4-trifluoromethyl-phenyl)-thiazol-2-yl]-propoxy}-phenyl)-2-ethoxy-propionic acid; 3-(2,5-Dimethyl-4-{3-[4-(4-trifluoromethyl-phenyl)-thiazol-2-yl]-propoxy}-phenyl)-2,2-dimethyl-propionic acid; 2-(2,5-Dimethyl-4-{3-[4-(4-trifluoromethyl-phenyl)-thiazol-2-yl]-propoxy}-phenylsulfanyl)-2-methyl-propionic acid; (2-Methyl-4-{2-[4-(4-trifluoromethylphenyl)-thiazol-2-yl]-ethoxy}-phenoxy)-acetic acid; (2-Methyl-4-{2-[4-(4-trifluoromethoxyphenyl)-thiazol-2-yl]-ethoxy}-phenoxy)-acetic acid; (2-Methyl-4-{2-[5-methyl-4-(4-trifluoromethyl-phenyl)-thiazol-2-yl]-ethoxy}-phenoxy)-acetic acid; (2-Methyl-4-{2-[4-(3-trifluoromethyl-phenyl)-thiazol-2-yl]-ethoxy}-phenoxy)-acetic acid; (4-{2-[4-(4-Methoxyphenyl)-thiazol-2-yl]-ethoxy}-2-methyl-phenoxy)-acetic acid; {2-Methyl-4-[2-(4-naphthalen-2-yl-thiazol-2-yl)-ethoxy]-phenoxy}-acetic acid; (2-Methyl-4-{3-[4-(4-trifluoromethyl-phenyl)-thiazol-2-yl]-propoxy}-phenoxy)-acetic acid; (2-Methyl-4-{3-[4-(4-trifluoromethoxy-phenyl)-thiazol-2-yl]-propoxy}-phenoxy)-acetic acid; (2-Methyl-4-{3-[5-methyl-4-(4-trifluoromethyl-phenyl)-thiazol-2-yl]-propoxy}-phenoxy)-acetic acid; (2-Methyl-4-{3-[4-(3-trifluoromethyl-phenyl)-thiazol-2-yl]-propoxy}-phenoxy)-acetic acid; (4-{3-[4-(4-Methoxy-phenyl)-thiazol-2-yl]-propoxy}-2-methyl-phenoxy)-acetic acid; {2-Methyl-4-[3-(4-naphthalen-2-yl-thiazol-2-yl)-propoxy]-phenoxy}-acetic acid; (2-Methyl-4-{4-[4-(4-trifluoromethyl-phenyl)-thiazol-2-yl]-butoxy}-phenoxy)-acetic acid; (2-Methyl-4-{4-[4-(4-trifluoromethoxy-phenyl)-thiazol-2-yl]-butoxy}-phenoxy)-acetic acid; (2-Methyl-4-{4-[5-methyl-4-(4-trifluoromethyl-phenyl)-thiazol-2-yl]-butoxy}-phenoxy)-acetic acid; (2-Methyl-4-{4-[4-(3-trifluoromethyl-phenyl)-thiazol-2-yl]-butoxy}-phenoxy)-acetic acid; (4-{4-[4-(4-Methoxy-phenyl)-thiazol-2-yl]-butoxy}-2-methyl-phenoxy)-acetic acid; {2-Methyl-4-[4-(4-naphthalen-2-yl-thiazol-2-yl)-butoxy]-phenoxy}-acetic acid; (5-Cyclopropyl-2-methyl-4-{2-[4-(4-trifluoromethyl-phenyl)-thiazol-2-yl]-ethoxy}-phenoxy)-acetic acid; {5-Cyclopropyl-2-methyl-4-[2-(4-naphthalen-2-yl-thiazol-2-yl)-ethoxy]-phenoxy}-acetic acid; (5-Cyclopropyl-2-methyl-4-{2-[5-methyl-4-(4-trifluoromethyl-phenyl)-thiazol-2-yl]-ethoxy}-phenoxy)-acetic acid; (3-{2-[4-(4-Trifluoromethyl-phenyl)-thiazol-2-yl]-ethoxy}-phenyl)-acetic acid; {3-[2-(4-Naphthalen-2-yl-thiazol-2-yl)-ethoxy]-phenyl}-acetic acid; (3-{2-[5-Methyl-4-(4-trifluoromethyl-phenyl)-thiazol-2-yl]-ethoxy}-phenyl)-acetic acid; (2-Cyclopropyl-3-{2-[4-(4-trifluoromethyl-phenyl)-thiazol-2-yl]-ethoxy}-phenyl)-acetic acid; {2-Cyclopropyl-3-[2-(4-naphthalen-2-yl-thiazol-2-yl)-ethoxy]-phenyl}-acetic acid; (2-Cyclopropyl-3-{2-[5-methyl-4-(4-trifluoromethyl-phenyl)-thiazol-2-yl]-ethoxy}-phenyl)acetic acid; (4-Cyclopropyl-3-{2-[4-(4-trifluoromethyl-phenyl)-thiazol-2-yl]-ethoxy}-phenyl)-acetic acid; {4-Cyclopropyl-3-[2-(4-naphthalen-2-yl-thiazol-2-yl)-ethoxy]-phenyl}-acetic acid; (4-Cyclopropyl-3-{2-[5-methyl-4-(4-trifluoromethyl-phenyl)-thiazol-2-yl]-ethoxy}-phenyl)-acetic acid; 2-Methyl-2-(4-{2-[4-(4-trifluoromethyl-phenyl)-thiazol-2-yl]-ethoxy}-phenoxy)-propionic acid; 2-Methyl-2-{4-[2-(4-naphthalen-2-yl-thiazol-2-yl)ethoxy]-phenoxy}-propionic acid; 2-Methyl-2-(4-{2-[5-methyl-4-(4-trifluoromethylphenyl)-thiazol-2-yl]-ethoxy}-phenoxy)-propionic acid; {4′-Methoxy-4-methyl-6-[4-(4-trifluoromethyl-phenyl)-thiazol-2-ylmethoxy]-biphenyl-3-yloxy}-acetic acid; {4-Methyl-4-trifluoromethoxy-6-[4-(4-trifluoromethyl-phenyl)-thiazol-2-ylmethoxy]-biphenyl-3-yloxy}-acetic acid; {4-Methyl-4′-methylsulfanyl-6-[4-(4-trifluoromethyl-phenyl)-thiazol-2-ylmethoxy]-biphenyl-3-yloxy}-acetic acid; {2-Methyl-4-[4-(4-trifluoromethyl-phenyl)-thiazol-2-ylmethoxy]-5-vinyl-phenoxy}-acetic acid; {5-Hex-1-enyl-2-methyl-4-[4-(4-trifluoromethyl-phenyl)-thiazol-2-ylmethoxy]-phenoxy}-acetic acid; {5-Furan-3-yl-2-methyl-4-[4-(4-trifluoromethyl-phenyl)-thiazol-2-ylmethoxy]-phenoxy}-acetic acid; {5-(6-Methoxy-pyridin-3-yl)-2-methyl-4-[4-(4-trifluoromethyl-phenyl)-thiazol-2-ylmethoxy]-phenoxy}-acetic acid; {4-Methyl-4′-propoxy-6-[4-(4-trifluoromethyl-phenyl)-thiazol-2-ylmethoxy]-biphenyl-3-yloxy}-acetic acid; {3′-Chloro-4′-methoxy-4-methyl-6-[4-(4-trifluoromethyl-phenyl)-thiazol-2-ylmethoxy]-biphenyl-3-yloxy}-acetic acid; {5-Benzo[1,3]dioxol-5-yl-2-methyl-4-[4-(4-trifluoromethyl-phenyl)-thiazol-2-ylmethoxy]-phenoxy}-acetic acid; {4′-Acetyl-4-methyl-6-[4-(4-trifluoromethyl-phenyl)-thiazol-2-ylmethoxy]-biphenyl-3-yloxy}-acetic acid; {4′-Dimethylamino-4-methyl-6-[4-(4-trifluoromethyl-phenyl)-thiazol-2-ylmethoxy]-biphenyl-3-yloxy}-acetic acid; {2-Methyl-5-thiophen-2-yl-4-[4-(4-trifluoromethyl-phenyl)-thiazol-2-ylmethoxy]-phenoxy}-acetic acid; {4-Methyl-4′-methylsulfanyl-6-[4-(4-trifluoromethoxy-phenyl)-thiazol-2-ylmethoxy]-biphenyl-3-yloxy}-acetic acid; {3′-Methoxy-4-methyl-6-[4-(4-trifluoromethoxy-phenyl)thiazol-2-ylmethoxy]-biphenyl-3-yloxy}-acetic acid; {2-Methyl-5-pyrrolidin-1-yl-4-[4-(4-trifluoromethyl-phenyl)-thiazol-2-ylmethoxy]-phenoxy}-acetic acid; (±)-(4′-Methoxy-4-methyl-6-{1-[4-(4-trifluoromethyl-phenyl)-thiazol-2-yl]-ethoxy}-biphenyl-3-yloxy)-acetic acid; (4′-Methoxy-4-methyl-6-{2-[4-(4-trifluoromethyl-phenyl)-thiazol-2-yl]-ethoxy}-biphenyl-3-yloxy)-acetic acid; (2-Methyl-4-{2-[4-(4-trifluoromethyl-phenyl)-thiazol-2-yl]-ethoxy}-5-vinyl-phenoxy)-acetic acid; {4′-Methoxy-4-methyl-6-[4-(4-nitro-phenyl)-thiazol-2-ylmethoxy]-biphenyl-3-yloxy}-acetic acid; {4′-Methoxy-6-[4-(4-methoxy-phenyl)-thiazol-2-ylmethoxy]-4-methyl-biphenyl-3-yloxy}-acetic acid; [4′-Methoxy-4-methyl-6-(4-p-tolyl-thiazol-2-ylmethoxy)-biphenyl-3-yloxy]-acetic acid; {6-[4-(4-Chloro-phenyl)-thiazol-2-ylmethoxy]-4′-methoxy-4-methyl-biphenyl-3-yloxy}-acetic acid; {6-[4-(4-Diethylaminophenyl)-thiazol-2-ylmethoxy]-4′-methoxy-4-methyl-biphenyl-3-yloxy}-acetic acid; {6-[4-(4-Cyano-phenyl)-thiazol-2-ylmethoxy]-4′-methoxy-4-methyl-biphenyl-3-yloxy}-acetic acid; [6-(4-Biphenyl-4-yl-thiazol-2-ylmethoxy)-4′-methoxy-4-methyl-biphenyl-3-yloxy]-acetic acid; [4′-Methoxy-4-methyl-6-(4-naphthalen-2-yl-thiazol-2-ylmethoxy)-biphenyl-3-yloxy]-acetic acid; {4′-Methoxy-6-[4-(3-methoxy-phenyl)-thiazol-2-ylmethoxy]-4-methyl-biphenyl-3-yloxy}-acetic acid; {5-Cyclopropyl-2-methyl-4-[4-(4-trifluoromethyl-phenyl)-thiazol-2-ylmethoxy]-phenoxy}-acetic acid; {5-Ethyl-2-methyl-4-[4-(4-trifluoromethyl-phenyl)-thiazol-2-ylmethoxy]-phenoxy}-acetic acid; [4-(4-Biphenyl-4-yl-thiazol-2-ylmethoxy)-5-cyclopropyl-2-methyl-phenoxy]-acetic acid; 2-(2,5-Dimethyl-4-{2-[4-(4-trifluoromethylphenyl)-thiazol-2-ylsulfanyl]-ethoxy}-phenoxy)-2-methyl-propionic acid; and 2-(2,5-Dimethyl-4-{3-[4-(4-trifluoromethyl-phenyl)-thiazol-2-ylsulfanyl]-propoxy}-phenoxy)-2-methyl-propionic acid.

Further preferred compounds of Formula I are detailed in the Examples, infra.

### Pharmacology and Utility

Compounds of the invention modulate the activity of PPARs and, as such, are useful for treating diseases or disorders in which PPARs contributes to the pathology and/or symptomology of the disease. This invention further provides compounds of this invention for use in the preparation of medicaments for the treatment of diseases or disorders in which PPARs contributes to the pathology and/or symptomology of the disease.

Such compounds may therefore be employed for the treatment of prophylaxis, dyslipidemia, hyperlipidemia, hypercholesteremia, atherosclerosis, atherogenesis, hypertriglyceridemia, heart failure, hyper cholesteremia, myocardial infarction, vascular diseases, cardiovascular diseases, hypertension, obesity, cachexia, HIV wasting syndrome, inflammation, arthritis, cancer, Alzheimer's disease, anorexia, anorexia nervosa, bulimia, skin disorders, respiratory diseases, ophthalmic disorders, IBDs (irritable bowel disease), ulcerative colitis and Crohn's disease. Preferably for the treatment of prophylaxis, dyslipidemia, hyperlipidemia, hypercholesteremia, atherosclerosis, atherogenesis, hypertriglyceridemia, cardiovascular diseases, hypertension, obesity, inflammation, cancer, skin disorders, IBDs (irritable bowel disease), ulcerative colitis and Crohn's disease.

Compounds of the invention can also be employed to treat long term critical illness, increase muscle mass and/or muscle strength, increase lean body mass, maintain muscle strength and function in the elderly, enhance muscle endurance and muscle function, and reverse or prevent frailty in the elderly.

Further, the compounds of the present invention may be employed in mammals as hypoglycemic agents for the treatment and prevention of conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome X. Preferably type-1 and type-2 diabetes, Impaired Glucose Metabolism (IGM), Impaired Glucose Tolerance (IGT) and Impaired Fasting Glucose (IFG).

In accordance with the foregoing, the present invention further provides a method for preventing or treating any of the diseases or disorders described above in a subject in need of such treatment, which method comprises administering to said subject a therapeutically effective amount (See, “Administration and Pharmaceutical Compositions”, infra) of a compound of the invention or a pharmaceutically acceptable salt thereof. For any of the above uses, the required dosage will vary depending on the mode of administration, the particular condition to be treated and the effect desired. The present invention also concerns: i) a compound of the invention or a pharmaceutically acceptable salt thereof for use as a medicament; and ii) the use of a compound of the invention or a pharmaceutically acceptable salt thereof for the manufacture of a medicament for preventing or treating any of the diseases or disorders described above.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrollidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

This invention also concerns a pharmaceutical composition comprising a therapeutically effective amount of a compound as described herein in combination with one or more pharmaceutically acceptable carriers.

Compounds of the invention can be administered in therapeutically effective amounts in combination with one or more therapeutic agents (pharmaceutical combinations).

Thus, the present invention also relates to pharmaceutical combinations, such as a combined preparation or pharmaceutical composition (fixed combination), comprising: 1) a compound of the invention as defined above or a pharmaceutical acceptable salt thereof; and 2) at least one active ingredient selected from:

a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; DPPIV (dipeptidyl peptidase IV) inhibitors such as DPP728, LAF237 (vildagliptin—Example 1 of WO 00/34241), MK-0431, saxagliptin, GSK23A; an AGE breaker; a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid described in the patent application WO 03/043985, as compound 19 of Example 4, a non-glitazone type PPARγ agonist e.g. GI-262570;

b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;

c) an anti-obesity agent or appetite regulating agent such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine or cannabinoid receptor antagonists;

d) anti-hypertensive agents, e.g., loop diuretics such as ethacrynic acid, furosemide and torsemide; diuretics such as thiazide derivatives, chlorithiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors;

e) a HDL increasing compound;

f) Cholesterol absorption modulator such as Zetia® and KT6-971;

g) Apo-A1 analogues and mimetics;

h) thrombin inhibitors such as Ximelagatran;

i) aldosterone inhibitors such as anastrazole, fadrazole, eplerenone;

j) Inhibitors of platelet aggregation such as aspirin, clopidogrel bisulfate;

k) estrogen, testosterone, a selective estrogen receptor modulator, a selective androgen receptor modulator;

l) a chemotherapeutic agent such as aromatase inhibitors e.g. femara, antiestrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib ({N-{5-[4-(4-methyl-piperazino-methyl)-benzoylamido]-2-methylphenyl}-4-(3-pyridyl)-2-pyrimidine-amine}) described in the European patent application EP-A-0 564 409 as example 21 or 4-Methyl-N-[3-(4-methyl-imidazol-1-yl)-5-trifluoromethyl-phenyl]-3-(4-pyridin-3-yl-pyrimidin-2-ylamino)-benzamide described in the patent application WO 04/005281 as example 92; and

m) an agent interacting with a 5-HT₃ receptor and/or an agent interacting with 5-HT₄ receptor such as tegaserod described in the U.S. Pat. No. 5,510,353 as example 13, tegaserod hydrogen maleate, cisapride, cilansetron;

or, in each case a pharmaceutically acceptable salt thereof; and optionally a pharmaceutically acceptable carrier.

Most preferred combination partners are tegaserod, imatinib, vildagliptin, metformin, a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid, a sulfonylurea receptor ligand, aliskiren, valsartan, orlistat or a statin such as pitavastatin, simvastatin, fluvastatin or pravastatin.

Preferably the pharmaceutical combinations contains a therapeutically effective amount of a compound of the invention as defined above, in a combination with a therapeutically effective amount of another therapeutic agent as described above, e.g., each at an effective therapeutic dose as reported in the art. Combination partners (1) and (2) can be administered together, one after the other or separately in one combined unit dosage form or in two separate unit dosage forms. The unit dosage form may also be a fixed combination.

The structure of the active agents identified by generic or trade names may be taken from the actual edition of the standard compendium “The Merck Index” or the Physician's Desk Reference or from databases, e.g. Patents International (e.g. IMS World Publications) or Current Drugs. The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

In another preferred aspect the invention concerns a pharmaceutical composition (fixed combination) comprising a therapeutically effective amount of a compound as described herein, in combination with a therapeutically effective amount of at least one active ingredient selected from the above described group a) to m), or, in each case a pharmaceutically acceptable salt thereof.

A pharmaceutical composition or combination as described herein for the manufacture of a medicament for the treatment of for the treatment of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, IBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.

Such therapeutic agents include estrogen, testosterone, a selective estrogen receptor modulator, a selective androgen receptor modulator, insulin, insulin derivatives and mimetics; insulin secretagogues such as the sulfonylureas, e.g., Glipizide and Amaryl; insulinotropic sulfonylurea receptor ligands, such as meglitinides, e.g., nateglinide and repaglinide; insulin sensitizers, such as protein tyrosine phosphatase-1B (PTP-1B) inhibitors, GSK3 (glycogen synthase kinase-3) inhibitors or RXR ligands; biguanides, such as metformin; alpha-glucosidase inhibitors, such as acarbose; GLP-1 (glucagon like peptide-1), GLP-1 analogs, such as Exendin-4, and GLP-1 mimetics; DPPIV (dipeptidyl peptidase IV) inhibitors, e.g. isoleucin-thiazolidide; DPP728 and LAF237, hypolipidemic agents, such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin, fluindostatin and rivastatin, squalene synthase inhibitors or FXR (liver X receptor) and LXR (farnesoid X receptor) ligands, cholestyramine, fibrates, nicotinic acid and aspirin. A compound of the present invention may be administered either simultaneously, before or after the other active ingredient, either separately by the same or different route of administration or together in the same pharmaceutical formulation.

The invention also provides for pharmaceutical combinations, e.g. a kit, comprising: a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I, in which R₄ is cyclic (e.g. cycloalkyl, heterocycloalkyl, aryl and heteroaryl), can be prepared by proceeding as in reaction schemes Ia and Ib:

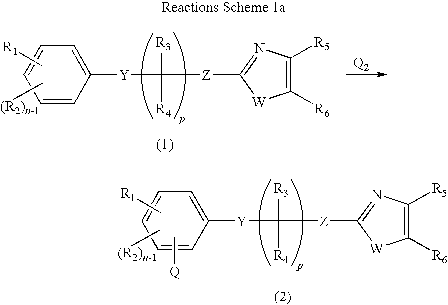

in which n, p, R₁, R₂, R₃, R₄, R₅, R₆, Y, Z and W are as defined for Formula I in the Summary of the Invention; and Q is a halogen, preferably Cl, I or Br. Compounds of 2 are formed by reacting a compound of formula I with a halogen (Q) (for example, Br₂, and the like) under suitable acidic conditions (for example, acetic acid, and the like), a suitable solvent (for example, dichloromethane, and the like). The reaction is carried out in the temperature range of about 10 to about 50° C. and takes up to about 12 hours to complete.

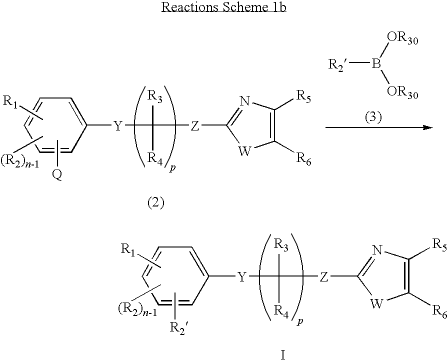

in which n, p, R₁, R₂, R₃, R₄, R₅, R₆, Y, Z and W are as defined for Formula I in the Summary of the Invention and R_{2′} is selected from C₂₋₆alkenyl, cyclopropyl, C₆₋₁₀aryl and C₅₋₁₀heteroaryl. Q is a halogen, preferably Cl, I or Br; and R³⁰ is independently selected from hydrogen, C₁₋₄alkyl or the R³⁰ radicals can be cyclized. Compounds of Formula I are prepared by reacting a compound of formula 2 with a compound of formula 3 in the presence of a suitable catalyst (e.g., Pd(Ph₃)₄, or the like), a suitable base (e.g., Na₂CO₃, or the like) and a suitable solvent (e.g., water, ethanol, DME or the like and mixtures thereof). The reaction is carried out in the temperature range of about 120 to about 200° C. (microwave) and takes up to about 20 minutes to complete or a corresponding time and temperature for conventional heating.

Compounds of Formula I, in which R₁ is defined by —X₁CR₉R₁₀X₂CO₂R₁₁ (shown below), —X₁SCR₉R₁₀X₂CO₂R₁₁ and —X₁OCR₉R₁₀X₂CO₂R₁₁, wherein R₇ is an alkyl group e.g., methyl or ethyl for a compound of formula 4 converting to hydrogen in formula I, can be prepared by proceeding as in reaction scheme 2:

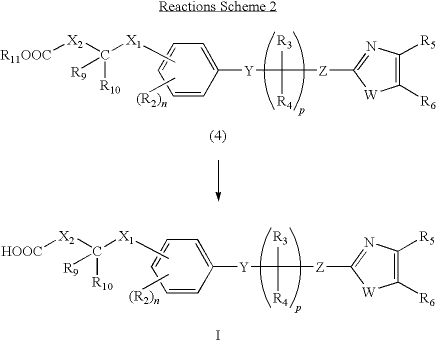

in which n, p, R₁, R₂, R₃, R₄, R₅, R₆, R₉, R₁₀, X₁, X₂, Y, Z and W are as defined for Formula I. Compounds of Formula I are prepared by reacting a compound of formula 4 in the presence of a suitable base (e.g., lithium hydroxide, or the like) and a suitable solvent (e.g., THF, water or the like). The reaction is carried out in the temperature range of about 0° C. to about 50° C. and takes up to about 30 hours to complete.

Compounds of Formula I can be prepared by proceeding as in reaction scheme 3:

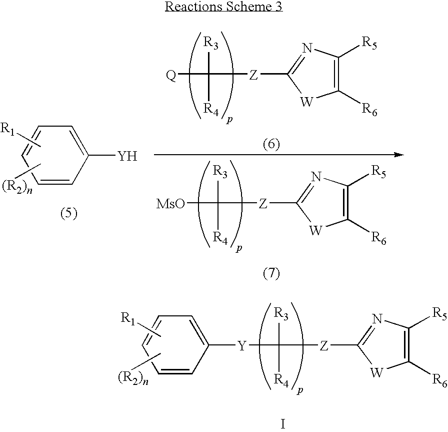

in which n, p, R₁, R₂, R₃, R₄, R₅, R₆, Y, Z and W are as defined for Formula I in the Summary of the Invention; and Q is a halogen, preferably Cl, I or Br. Compounds of I are formed by reacting a compound of formula 5 with a compound of formula 6 or formula 7 (where OMs is a mesylate formed from the corresponding alcohol using mesylation conditions known to one of skill in the art). The reaction proceeds in the presence of a suitable solvent (for example, acetonitrile, acetone, and the like), a suitable inorganic base (for example, Cs₂CO₃, and the like). The reaction is carried out in the temperature range of about 10 to about 80° C. and takes up to about 12 hours to complete.

Compounds of Formula I can be prepared by proceeding as in reaction scheme 4:

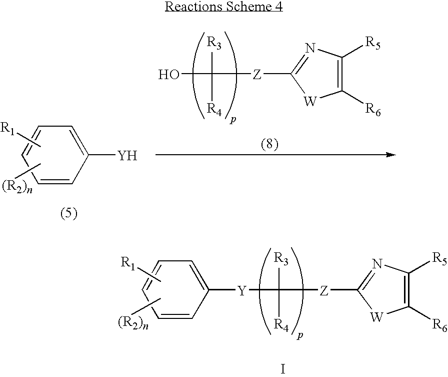

in which n, p, R₁, R₂, R₃, R₄, R₅, R₆, Y, Z and W are as defined for Formula I in the Summary of the Invention. Compounds of formula I are formed by reacting a compound of formula 5 with a compound of formula 8. The reaction proceeds in the presence of a suitable solvent (for example, DCM, THF, and the like), suitable activation reagents (for example, triphenylphosphine/diethylazodicarboxylate, and the like). The reaction is carried out in the temperature range of about 0 to about 30° C. and takes up to about 12 hours to complete.

Compounds of Formula I can be prepared by proceeding as in reaction scheme 5:

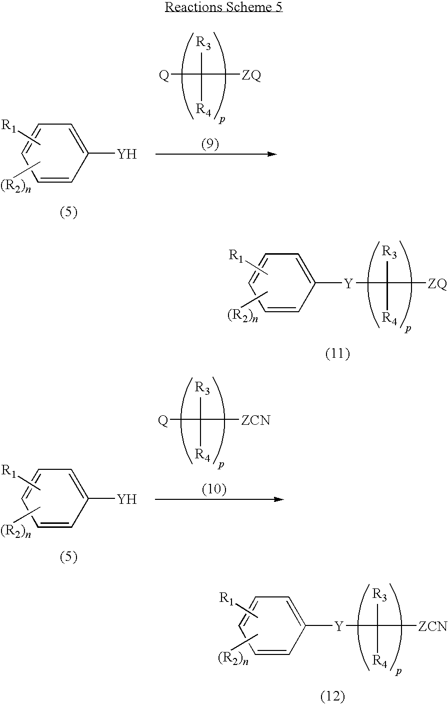

in which n, p, R₁, R₂, R₃, R₄, Y and Z are as defined for Formula I in the Summary of the Invention; and Q is a halogen, preferably Cl, I or Br. Compounds of formula 11 and 12 are formed by reacting a compound of formula 5 with a compound of formula 9 or 10, respectively. The reaction proceeds in the presence of a suitable solvent (for example, acetonitrile, acetone, and the like), a suitable inorganic base (for example, Cs₂CO₃, K₂CO₃, and the like). The reaction is carried out in the temperature range of about 10 to about 100° C. and takes up to about 24 hours to complete.

Compounds of Formula I can be prepared by proceeding as in reaction scheme 6:

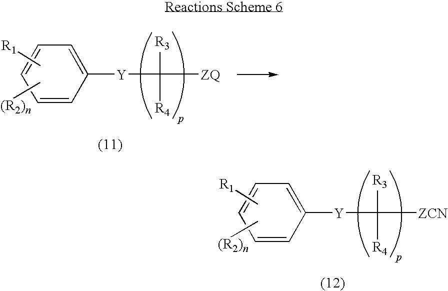

in which n, p, R₁, R₂, R₃, R₄, Y and Z are as defined for Formula I in the Summary of the Invention; and Q is a halogen, preferably Cl, I or Br. Compounds of formula 12 are formed by reacting a compound of formula 11 with a suitable reactant (for example, sodium cyanide, potassium cyanide, and the like), in a suitable solvent (for example, DMF, DMSO, ethanol, water, and the like). The reaction is carried out in the temperature range of about 0 to about 100° C. and takes up to about 12 hours to complete.

Compounds of Formula I can be prepared by proceeding as in reaction scheme 7a and 7b:

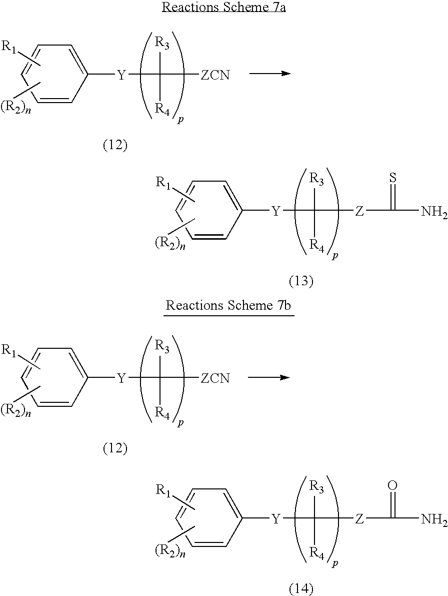

n which n, p, R₁, R₂, R₃, R₄, Y and Z are as defined for Formula I in the Summary of the Invention. Compounds of formula 13 are formed by reacting a compound of formula 12 with a suitable solvent (for example, DMF, and the like) and a suitable reagent (for example, thioacetamide, and the like) under suitable acidic conditions (for example, HCl, and the like). The reaction is carried out in the temperature range of about 0 to about 120° C. and takes up to about 6 hours to complete.

Alternatively, compounds of formula 13 are formed by reacting a compound of formula 12 with a suitable solvent (for example, pyridine, and the like) and a suitable reagent (for example, H₂S, and the like) under suitable basic conditions (for example, triethylamine, and the like). The reaction is carried out in the temperature range of about 0 to about 50° C. and takes up to about 6 hours to complete.

Compounds of formula 14 are formed by reacting a compound of formula 12 with a suitable solvent (for example, methanol, ethanol, and the like) and a suitable reagent (for example, H₂SO₄/water, and the like). The reaction is carried out in the temperature range of about 0 to about 50° C. and takes up to about 24 hours to complete.

Compounds of Formula I can be prepared by proceeding as in reaction scheme 8:

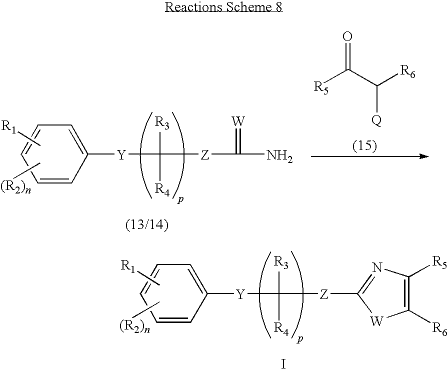

in which n, p, R₁, R₂, R₃, R₄, R₅, R₆, Y, Z and W are as defined for Formula I in the Summary of the Invention; and Q is a halogen, preferably Cl, I or Br. Compounds of formula I are formed by reacting a compound of formula 13 or 14 with a compound of formula 15 optionally in the presence of a suitable solvent (for example, ethanol, and the like). The reaction is carried out in the temperature range of about 50 to about 120° C. and takes up to about 24 hours to complete.

Detailed reaction conditions are described in the examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction schemes 1 to 8; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following intermediates and examples that illustrate the preparation of compounds of Formula I according to the invention.

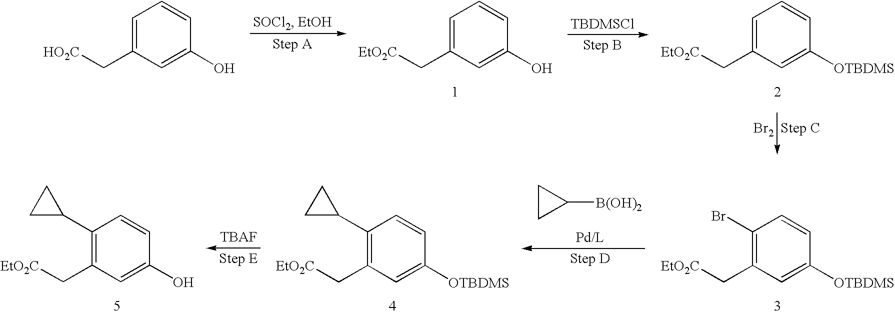

### Intermediate 5

### (2-Cyclopropyl-5-hydroxy-phenyl)-acetic Acid Ethyl Ester

Step A: (3-Hydroxy-phenyl)-acetic acid (10 g, 65.7 mmol) is dissolved in EtOH (50 mL). Catalytic amounts of thionyl chloride (˜0.5 mL) are added and the solution is stirred for 6 h at rt. The solvent is removed in vacuo to give (3-hydroxy-phenyl)-acetic acid ethyl ester 1 (11.8 g, quant.): MS calcd. for C₁₀H₁₃O₃ (M+H⁺) 181.1, found 181.0.

Step B: (3-hydroxy-phenyl)-acetic acid ethyl ester 1 (5.93 g, 32.9 mmol) and imidazole (6.72 g, 98.7 mmol) are dissolved in DMF (16 mL) and stirred at rt for 10 min. Then TBDMSCl (7.44 g, 49.4 mmol) dissolved in DMF (4 mL) is added slowly and the mixture is stirred at rt overnight. Then water (50 mL) is added and the mixture is extracted with ether twice. The organic layers are combined, washed with water and brine, dried over MgSO₄, filtered and concentrated to give [3-(tert-butyl-dimethyl-silanyloxy)-phenyl]-acetic acid ethyl ester 2 as an oil: ¹H-NMR (400 MHz, CDCl₃) δ=6.97 (t, J=7.8 Hz, 1H), 6.67 (d, J=7.6 Hz, 1H), 6.59 (s, 1H), 6.54 (d, J=8.1 Hz, 1H), 3.95 (q, J=7.1 Hz, 2H), 3.35 (s, 2H), 1.05 (t, J=7.1 Hz, 3H), 0.79 (s, 9H), 0.00 (s, 6H); MS calcd. for C₁₆H₂₇O₃Si (M+H⁺) 295.2, found 295.1.

Step C: [3-(tert-Butyl-dimethyl-silanyloxy)-phenyl]-acetic acid ethyl ester 2 (9.20 g, 31.2 mmol) and potassium acetate (3.10 g, 31.2 mmol) are dissolved in acetic acid (120 mL) and cooled to 15° C. Bromine (1.60 mL, 31.2 mmol) dissolved in HOAc (60 mL) is added at a rate that kept the temperature at approx. 15° C., then the mixture is stirred at this temperature for 2 h. Insoluble salts are filtered and the solution is concentrated. The remainder is taken up in ether and washed with saturated bicarbonate, water and brine. The organic layer is dried over MgSO₄, filtered and concentrated. The remainder is purified by flash chromatography (EtOAc/Hexanes gradient) to afford [2-bromo-5-(tert-butyl-dimethyl-silanyloxy)-phenyl]-acetic acid ethyl ester 3 as a colourless oil: ¹H-NMR (400 MHz, CDCl₃) δ=7.19 (d, J=8.6 Hz, 1H), 6.61 (d, J=2.9 Hz, 1H), 6.44 (dd, J=8.6 Hz, J=2.9 Hz, 1H), 3.99 (q, J=7.1 Hz, 2H), 3.51 (s, 2H), 1.07 (t, J=7.1 Hz, 3H), 0.78 (s, 9H), 0.00 (s, 6H); MS calcd. for C₁₆H₂₆O₃BrSi (M+H⁺) 373.1, found 373.0.

Step D: [2-Bromo-5-(tert-butyl-dimethyl-silanyloxy)-phenyl]-acetic acid ethyl ester 3 (1.00 g, 2.68 mmol), potassium phosphate (1.99 g, 9.38 mmol) and cyclopropylboronic acid (0.35 g, 4.02 mmol) are dissolved in toluene (12 mL). Tricyclohexylphosphine (0.23 g, 0.80 mmol), palladium acetate (0.09 g, 0.40 mmol) and water (0.6 mL) are added and the mixture is heated to 100° C. overnight. Then the mixture is diluted with EtOAc (160 mL) and washed with water and brine successively. The organic layer is dried over MgSO₄, filtered and concentrated to afford crude [5-(tert-butyl-dimethyl-silanyloxy)-2-cyclopropyl-phenyl]-acetic acid ethyl ester 4 as a colourless oil: ¹H-NMR (400 MHz, CDCl₃) δ=6.91 (d, J=8.3 Hz, 1H), 6.72 (d, J=2.6 Hz, 1H), 6.65 (dd, J=8.3 Hz, J=2.6 Hz, 1H), 4.15 (q, J=7.1 Hz, 2H), 3.76 (s, 2H), 1.84 (m, 1H), 1.24 (t, J=7.1 Hz, 3H), 0.97 (s, 9H), 0.87 (m, 2H), 0.57 (m, 2H), 0.00 (s, 6H); MS calcd. for C₁₉H₃₁O₃Si (M+H⁺) 335.2, found 335.1.

Step E: Crude [5-(tert-butyl-dimethyl-silanyloxy)-2-cyclopropyl-phenyl]-acetic acid ethyl ester 4 is dissolved in a mixture of THF (5 mL) and TBAF (5 mL) and stirred at rt for 90 min. Water (75 mL) is added and the mixture is extracted with EtOAc (100 mL) twice. The organic layers are combined, washed with 0.1 M HCl and brine, dried over MgSO₄, filtered and concentrated. The remainder is purified by reverse phase HPLC (H₂O/MeCN gradient) to afford (2-cyclopropyl-5-hydroxy-phenyl)-acetic acid ethyl ester 5 as an oil: ¹H-NMR (400 MHz, CDCl₃) δ=6.92 (d, J=8.3 Hz, 1H), 6.71 (d, J=2.6 Hz, 1H), 6.64 (dd, J=8.3 Hz, J=2.6 Hz, 1H), 4.16 (q, J=7.1 Hz, 2H), 3.76 (s, 2H), 1.80 (m, 1H), 1.25 (t, J=7.1 Hz, 3H), 0.85 (m, 2H), 0.55 (m, 2H); MS calcd. for C₁₃H₁₇O₃ (M+H⁺) 221.1, found 221.0.

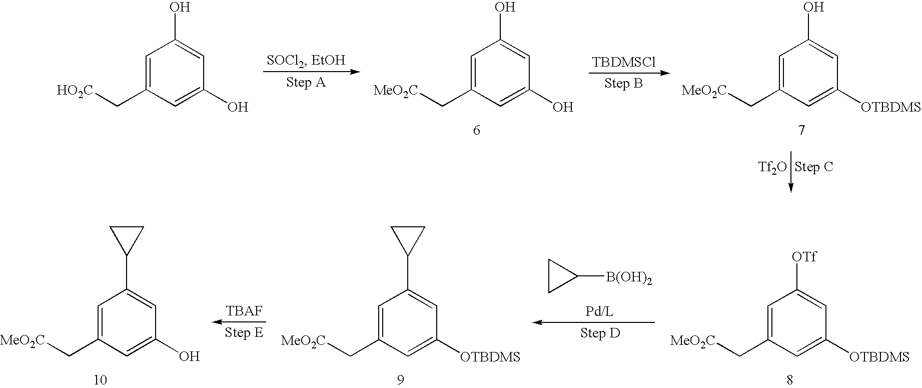

### Intermediate 10

### (3-Cyclopropyl-5-hydroxy-phenyl)-acetic Acid Methyl Ester

Step A: (3,5-Dihydroxy-phenyl)-acetic acid (5 g, 29.7 mmol) is dissolved in MeOH (30 mL). Catalytic amounts of thionyl chloride (˜0.25 mL) are added and the solution is stirred at rt overnight. The solvent is removed in vacuo to give (3,5-dihydroxyphenyl)-acetic acid methyl ester 6 (5.44 g, quant.): MS calcd. for C₉H₁₁O₄ (M+H⁺) 183.1, found 183.0.

Step B: (3,5-Dihydroxy-phenyl)-acetic acid methyl ester 6 (2.50 g, 13.9 mmol) and imidazole (3.78 g, 55.5 mmol) are dissolved in DMF (10 mL) and stirred at rt for 10 min. Then TBDMSCl (1.67 g, 11.1 mmol) dissolved in DMF (4 mL) is added slowly and the mixture is stirred at rt for 8 h. Then water (50 mL) is added and the mixture is extracted with ether twice. The organic layers are combined, washed with water and brine, dried over MgSO₄, filtered and concentrated. The crude product is dissolved in DCM/exanes 1:9 and filtered to give a mixture of bisilylated side product and [3-(tert-butyl-dimethyl-silanyloxy)-5-hydroxy-phenyl]-acetic acid methyl ester 7 as a colourless oil: MS calcd. for C₁₅H₂₅O₄Si (M+H⁺) 297.1, found 297.1.

Step C: [3-(tert-Butyl-dimethyl-silanyloxy)-5-hydroxy-phenyl]-acetic acid methyl ester 7 (1.81 g, 6.1 mmol) and triethyl amine (0.85 mL, 6.1 mmol) are dissolved in DCM (30 mL) and cooled to 0° C. Triflic anhydride (1.03 mL, 6.1 mmol) dissolved in DCM (20 mL) is added dropwise, then the mixture is stirred at 0° C. for 3 h. The solution is washed with saturated bicarbonate, water and brine. The organic layer is dried over MgSO₄, filtered and concentrated. The remainder is purified by flash chromatography (EtOAc/Hexanes gradient) to afford [3-(tert-butyl-dimethyl-silanyloxy)-5-trifluoromethanesulfonyloxy-phenyl]-acetic acid methyl ester 8 as a colourless oil: ¹H-NMR (400 MHz, CDCl₃) δ=6.61 (s, 1H), 6.58 (s, 1H), 6.44 (s, 1H), 3.49 (s, 3H), 3.37 (s, 2H), 0.76 (s, 9H), 0.00 (s, 6H); MS calcd. for C₁₆H₂₄F₃O₆SSi (M+H⁺) 429.1, found 429.1.

Step D: [3-(tert-Butyl-dimethyl-silanyloxy)-5-trifluoromethanesulfonyloxy-phenyl]-acetic acid methyl ester 8 (0.5 g, 1.13 mmol), potassium phosphate (0.84 g, 3.96 mmol) and cyclopropylboronic acid (0.13 g, 1.472 mmol) are dissolved in toluene (6 mL). Tricyclohexylphosphine (32 mg, 0.11 mmol), palladium acetate (13 mg, 0.06 mmol) and water (0.3 mL) are added and the mixture is heated to 100° C. overnight. Then the mixture is diluted with EtOAc (100 mL) and washed with water and brine successively. The organic layer is dried over MgSO₄, filtered and concentrated to afford crude [3-(tert-butyl-dimethyl-silanyloxy)-5-cyclopropyl-phenyl]-acetic acid methyl ester 9 as a colourless oil: MS calcd. for C₁₈H₂₉O₃Si (M+H⁺) 321.2, found 321.1.

Step E: Crude [3-(tert-butyl-dimethyl-silanyloxy)-5-cyclopropyl-phenyl]-acetic acid methyl ester 9 (0.22 g, 0.69 mmol) is dissolved in a mixture of THF (5 mL) and TBAF (5 mL) and stirred at rt for 90 min. Water (75 mL) is added and the mixture is extracted with EtOAc (100 mL) twice. The organic layers are combined, washed with 0.1 M HCl and brine, dried over MgSO₄, filtered and concentrated. The remainder is purified by reverse phase HPLC (H₂O/MeCN gradient) to afford (3-Cyclopropyl-5-hydroxy-phenyl)acetic acid methyl ester 10 as an oil: ¹H-NMR (400 MHz, CDCl₃) δ=6.56 (s, 1H), 6.55 (s, 1H), 6.43 (s, 1H), 3.69 (s, 3H), 3.53 (s, 2H), 1.81 (m, 1H), 0.92 (m, 2H), 0.66 (m, 2H); MS calcd. for C₂H₁₅O₃ (M+H⁺) 207.1, found 207.1.

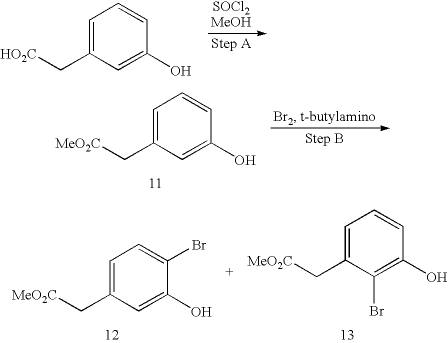

### Intermediates 12 and 13

### (4-Bromo-3-hydroxy-phenyl)-acetic Acid Methyl Ester and (2-bromo-3-hydroxy-phenyl)-acetic Acid Methyl Ester

Step A: (3-Hydroxy-phenyl)-acetic acid (3.0 g, 19.7 mmol) is dissolved in MeOH (50 mL). Catalytic amounts of thionyl chloride (˜0.1 mL) are added and the solution is stirred for 6 h at rt. The solvent is removed in vacuo to give (3-hydroxy-phenyl)-acetic acid methyl ester 11 (3.2 g, quant.): MS calcd. for C₉H₁₁O₃ (M+H⁺) 167.1, found 167.0.

Step B: tert-Butylamine (5 mL, 48 mmol) is dissolved in toluene (40 mL) and cooled to −30° C., then bromine (1.2 mL, 24 mmol) is added dropwise and stirred at −30° C. for 0.5 h. The mixture is cooled to −78° C. and a solution of (3-hydroxy-phenyl)-acetic acid methyl ester 11 (4 g, 24 mmol) in DCM (20 mL) is added dropwise and stirred at rt for 16 h. 1 N HCl (20 mL) is added and the mixture is extracted with DCM (50 mL) and washed with a saturated solution of NaHCO₃ (50 mL), then brine (20 mL). The organic layer is dried over MgSO₄, filtered and concentrated. The regioisomers are separated and purified by reverse phase HPLC (H₂O/MeCN gradient) to afford 12 (4-bromo-3-hydroxy-phenyl)-acetic acid methyl ester: ¹H-NMR (400 MHz, CDCl₃) δ=7.39 (d, J=8.4 Hz, 1H), 6.95 (d, J=2.0 Hz, 1H), 6.73 (dd, J=2.0, 8.4 Hz, 1H), 3.70 (s, 3H), 3.55 (s, 2H); MS calcd. for C₉H₁₀BrO₃ (M+H⁺) 244.9, found 245.0 and 13 (2-bromo-3-hydroxy-phenyl)-acetic acid methyl ester: ¹H-NMR (400 MHz, CDCl₃) δ=7.17 (t; J=8.0 Hz, 1H), 6.94 (dd, J=1.2, 8.0 Hz, 1H), 6.86 (dd, J=1.2, 8.0 Hz, 1H), 3.79 (s, 2H), 3.72 (s, 3H); MS calcd. for C₉H₁₀BrO₃ (M+H⁺) 244.9, found 245.0.

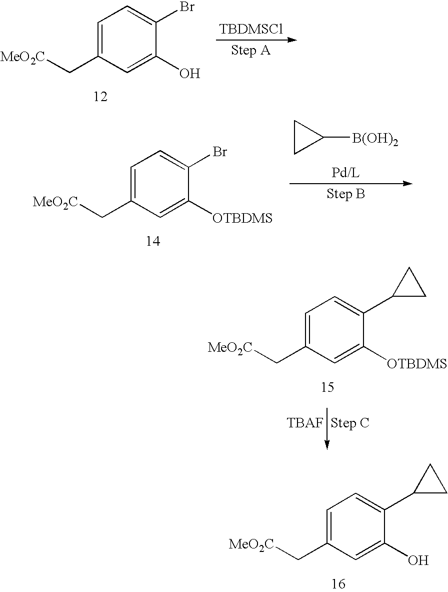

### Intermediate 16

### (4-Cyclopropyl-3-hydroxy-phenyl)-acetic Acid Methyl Ester

Step A: (4-Bromo-3-hydroxy-phenyl)-acetic acid methyl ester 12 (751 mg, 2.09 mmol) and TBDMSCl (346 mg, 2.30 mmol) are dissolved in DCM (4 mL). Triethylamine (0.44 mL, 3.13 mmol) and DMAP (25 mg, 0.21 mmol) are added and the mixture is stirred at rt for 2 h. Water (10 mL) is added and the mixture is extracted with DCM. The organic layer is washed with 1 N HCl and brine, dried over MgSO₄, filtered, concentrated and purified by flash chromatography (EtOAc/Hexanes gradient) to afford [4-bromo-3-(tert-butyl-dimethyl-silanyloxy)-phenyl]-acetic acid methyl ester 14 as an oil: MS calcd. for C₁₅H₂₄BrO₃Si (M+H⁺) 359.1, found 359.0.

Step B: [4-Bromo-3-(tert-butyl-dimethyl-silanyloxy)-phenyl]-acetic acid methyl ester 14 (663 mg, 1.85 mmol), potassium phosphate (1.37 g, 6.47 mmol) and cyclopropylboronic acid (0.19 g, 2.22 mmol) are dissolved in toluene (40 mL). Tricyclohexylphosphine (42 mg, 0.18 mmol), palladium acetate (26 mg, 0.09 mmol) and water (2 mL) are added and the mixture is heated to 100° C. overnight. The mixture is diluted with EtOAc (160 mL) and washed with water and brine successively. The organic layer is dried over MgSO₄, filtered, concentrated and purified by flash chromatography (EtOAc/Hexanes gradient) to afford [3-(tert-butyl-dimethyl-silanyloxy)-4-cyclopropyl-phenyl]-acetic acid methyl ester 15 as a colourless oil: ¹H-NMR (400 MHz, CDCl₃) δ=6.77 (d, J=2.4 Hz, 1H), 6.73 (m, 2H), 3.67 (s, 3H), 3.53 (s, 2H), 2.10 (m, 1H), 1.03 (s, 9H), 0.89 (m, 2H), 0.61 (m, 2H), 0.23 (s, 6H); MS calcd. for C₁₈H₂₉O₃Si (M+H⁺) 321.2, found 321.1.

Step C: [3-(tert-Butyl-dimethyl-silanyloxy)-4-cyclopropyl-phenyl]-acetic acid methyl ester 15 (479 mg, 1.49 mmol) is dissolved in a mixture of THF (20 mL) and TBAF (1.8 mL, 1.79 mmol) and stirred at rt for 90 min. 1 N HCl (40 mL) is added and the mixture is extracted with EtOAc (40 mL). The organic layer is washed with 1 N HCl and brine, dried over MgSO₄, filtered and concentrated to afford (4-cyclopropyl-3-hydroxyphenyl)-acetic acid methyl ester 16 (0.42 g, quant.) as an oil: ¹H-NMR (400 MHz, CDCl₃) 5=7.00 (d, J=7.6 Hz, 1H), 6.79 (d, J=1.6 Hz, 1H), 6.75 (dd, J=1.6, 7.6 Hz, 1H), 3.69 (s, 3H), 3.55 (s, 2H), 1.79 (m, 1H), 0.95 (m, 2H), 0.63 (m, 2H). MS calcd. for C₁₂H₁₅O₃ (M+H⁺) 207.1, found 207.0.

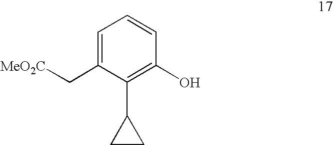

### Intermediate 17

### (2-Cyclopropyl-3-hydroxy-phenyl)-acetic Acid Methyl Ester

Following the procedure for Intermediate 16, except substituting bromide 13 for bromide 12, the title compound is prepared as a clear liquid: ¹H-NMR (400 MHz, CDCl₃) δ=7.12 (t, J=7.6 Hz, 1H), 6.82 (dd, J=1.2, 8.4 Hz, 1H), 6.78 (dd, J=1.2, 7.6 Hz, 1H), 3.86 (s, 2H), 3.70 (s, 3H), 1.62 (m, 1H), 1.10 (m, 2H), 0.63 (m, 2H). MS calcd. for C₁₂H₁₅O₃ (M+H⁺) 207.1, found 207.0.

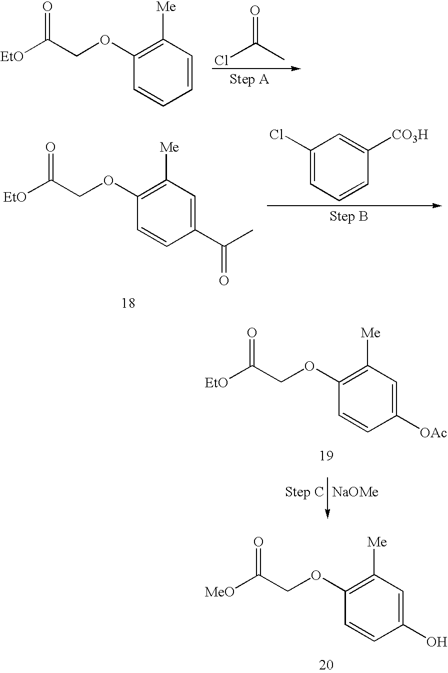

### Intermediate 20

### (4-Hydroxy-2-methyl-phenoxy)-acetic Acid Methyl Ester

Step A: (2-Methylphenoxy)acetic acid ethyl ester (66.03 g, 340 mmol) is dissolved in dichloroethane (400 mL). Aluminum chloride (100.02 g, 750 mmol) is added and the light-brown mixture is stirred for 10 minutes at rt. Acetyl chloride (35 mL, 493 mmol) is added dropwise using an addition funnel. The rate of addition is adjusted to maintain a relatively slow emission of hydrogen chloride gas. The resulting dark brown solution is allowed to cool off to rt, then is poured over 300 g of crushed ice. The mixture is diluted with DCM (300 mL) and washed successively with water, saturated NaHCO₃ solution, water, saturated NH₄Cl solution, and brine. The organic layer is dried over Na₂SO₄, filtered and concentrated to afford 18 as a brown oil that solidified as a crystalline mass: ¹H-NMR (400 MHz, CDCl₃) δ=7.79 (d, J=2.0 Hz, 1H), 7.77 (dd, J=2.0, 8.4 Hz, 1H), 6.69 (d, J=8.4 Hz, 1H), 4.71 (s, 2H), 4.26 (q, J=7.2 Hz, 2H), 2.54 (s, 3H), 2.32 (s, 2H), 1.29 (t, J=7.2 Hz, 3H).

Step B: (4-Acetyl-2-methyl-phenoxy)-acetic acid ethyl ester 18 (76.5 g, 324 mmol), 77% mCPBA (100.3 g, 407 mmol) and p-TsOH (13 g, 68 mmol) in dichloroethane (450 mL) are heated to 50° C. for 30 h. The reaction mixture is then washed with 1 M KI (2×500 mL) and NaHSO₃ (2×500 mL). The organic layer is dried (MgSO₄), filtered and concentrated to afford 19 as a brown syrup.

Step C: A solution of (4-acetoxy-2-methyl-phenoxy)-acetic acid ethyl ester 19 (from step B above) in dry MeOH (400 mL) is combined with a 0.5 M solution of NaOMe in MeOH (650 mL, 325 mmol) and stirred for 2 h at rt. The solution is neutralized with 1 M HCl and washed with H₂O (2×500 mL). The organic layer is dried (Na₂SO₄), filtered and concentrated to afford 20 as a light-brown solid: ¹H-NMR (400 MHz, CDCl₃) δ=6.58 (d, J=2.8 Hz, 1H), 6.54 (d, J=8.4 Hz), 6.50 (dd, J=2.8, 8.4 Hz, 1H), 4.7 (br. s, 1H), 4.54 (s, 2H), 3.73 (s, 3H), 2.17 (s, 3H). MS calcd. for C₁₀H₃₀O₄ (M+H⁺) 197.1, found 197.4.

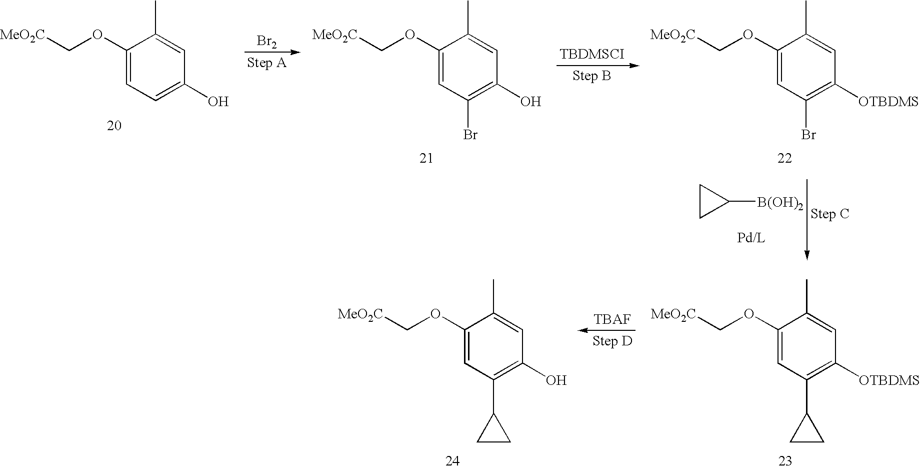

### Intermediate 24

### (5-Cyclopropyl-4-hydroxy-2-methyl-phenoxy)-acetic Acid Methyl Ester

Step A: Intermediate 20 (5 g, 26 mmol) is dissolved in DCM (100 mL). Bromine (1.44 mL, 28 mmol) in DCM (20 mL) is added dropwise and stirred at rt for 2 h. The mixture is washed with aqueous saturated NaHCO₃ (2×100 mL) and brine (20 mL), dried over MgSO₄, filtered, and concentrated. The residue is recrystallized from EtOAc/Hexanes to afford 21 (5-bromo-4-hydroxy-2-methyl-phenoxy)-acetic acid methyl ester: ¹H-NMR (400 MHz, CDCl₃) δ=7.01 (s, 1H), 6.98 (s, 1H), 5.31 (s, 1H), 4.74 (s, 2H), 3.97 (s, 3H), 2.39 (s, 3H); MS calcd. for C₁₀H₁₂BrO₄ (M+H⁺) 275.0, found 297.0 (M+Na⁺).

Step B: ((5-Bromo-4-hydroxy-2-methyl-phenoxy)-acetic acid methyl ester 21 (25.5 mmol) and TBDMSCl (4.23 g, 28.0 mmol) are dissolved in DCM (100 mL). Triethylamine (5.4 mL, 38.2 mmol) and DMAP (311 mg, 2.5 mmol) are added and the mixture is stirred at rt for 2 h. The mixture is washed with 1 N HCl and brine, dried over MgSO₄, filtered, concentrated and purified by flash chromatography (EtOAc/Hexanes gradient) to afford [5-bromo-4-(tert-butyl-dimethyl-silanyloxy)-2-methyl-phenoxy]-acetic acid methyl ester 22 as an oil: MS calcd. for C₁₆H₂₆BrO₄Si (M+H⁺) 389.1, found 389.0.

Step C: [5-Bromo-4-(tert-butyl-dimethyl-silanyloxy)-2-methyl-phenoxy]-acetic acid methyl ester 22 (2.75 g, 7.0 mmol), potassium phosphate (5.2 g, 24.5 mmol) and cyclopropylboronic acid (0.72 g, 8.4 mmol) are dissolved in toluene (80 mL). Tricyclohexylphosphine (157 mg, 0.7 mmol), palladium acetate (98 mg, 0.35 mmol) and water (4 mL) are added and the mixture is heated to 100° C. overnight. The mixture is diluted with EtOAc (140 mL) and washed with water and brine successively. The organic layer is dried over MgSO₄, filtered, and concentrated to afford crude [4-(tert-butyl-dimethyl-silanyloxy)-5-cyclopropyl-2-methyl-phenoxy]-acetic acid methyl ester 23 which is used directly in the next step: MS calcd. for C₁₉H₃₁O₄Si (M+H⁺) 351.2, found 351.2.

Step D: [4-(tert-Butyl-dimethyl-silanyloxy)-5-cyclopropyl-2-methyl-phenoxy]-acetic acid methyl ester 23 (1.0 g, 3.1 mmol) is dissolved in a mixture of THF (30 mL) and TBAF (3.7 mL, 3.7 mmol) and stirred at rt for 90 min. 1 N HCl (40 mL) is added and the mixture is extracted with EtOAc (40 mL). The organic layer is washed with 1 N HCl and brine, dried over MgSO₄, filtered and concentrated. The residue is triturated with hexanes to afford (5-cyclopropyl-4-hydroxy-2-methyl-phenoxy)acetic acid methyl ester 24 as an off-white powder: ¹H-NMR (400 MHz, CDCl₃) δ=6.67 (s, 1H), 6.50 (s, 1H), 4.56 (s, 2H), 3.80 (s, 3H), 2.22 (s, 3H), 1.76 (m, 1H), 0.94 (m, 2H), 0.59 (m, 2H). MS calcd. for C₁₃H₁₇O₄ (M+H⁺) 237.1, found 237.0.

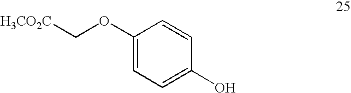

### Intermediate 25

### (4-Hydroxy-phenoxy)-acetic Acid Methyl Ester

(4-Hydroxy-phenoxy)-acetic acid (10.98 g, 65.3 mmol) is dissolved in methanol (50 mL). Catalytic concentrated sulfuric acid (0.2 mL) is added and the mixture is heated to reflux overnight. Cooling, treatment with solid NaHCO₃ and activated charcoal, drying over MgSO₄, filtration and concentration yielded a white solid (12.86 g, quant.): ¹H-NMR (400 MHz, CDCl₃) δ=7.80 (d, J=9.2 Hz, 2H), 6.75 (d, J=9.2 Hz, 2H), 4.58 (s, 2H), 3.80 (s, 3H). MS calcd. for C₉H₁₁O₄ (M+H⁺) 183.1, found 183.0.

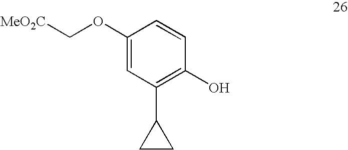

### Intermediate 26

### (3-Cyclopropyl-4-hydroxy-phenoxy)-acetic Acid Methyl Ester

Following the procedure for intermediate 24, except substituting intermediate 25 for intermediate 20 in step A, the title compound is prepared as a clear solid: ¹H-NMR (400 MHz, CDCl₃) δ=6.77 (dd, J=1.2, 7.6 Hz, 1H), 6.68 (m, 2H), 4.56 (s, 2H), 3.80 (s, 3H), 1.81 (m, 1H), 0.96 (m, 2H), 0.64 (m, 2H). MS calcd. for C₁₂H₁₅O₄ (M+H⁺) 223.1, found 223.0.

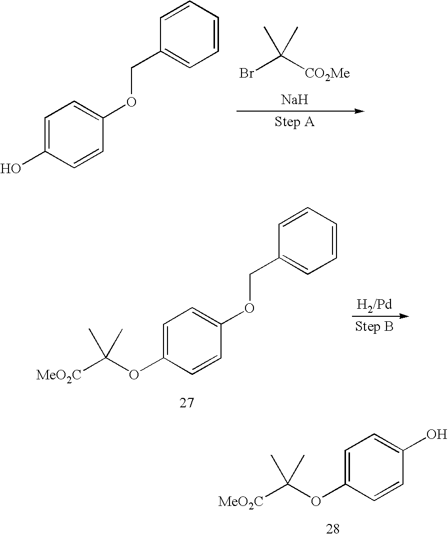

### Intermediate 28

### 2-(4-Hydroxy-phenoxy)-2-methyl-propionic Acid Methyl Ester

Step A: 4-(Benzyloxy)phenol (5.0 g, 25 mmol) is dissolved in DMF (40 mL). To the solution is added NaH (60% dispersion, 1.1 g, 27.5 mmol) in portions while it is kept at rt. After stirring the suspension for 30 min at rt methyl-α-bromoisobutyrate (9.05 g, 50 mmol) is added dropwise. The mixture is stirred at 50° C. for 3 h, then concentrated. The remainder is diluted with water (200 mL) and extracted with EtOAc (3×150 mL). The organic layer is separated and dried over MgSO₄, filtered and concentrated. The crude product is purified by flash chromatography (silica, Hex/EtOAc gradient) to afford 2-(4-benzyloxy-phenoxy)-2-methyl-propionic acid methyl ester 27 as a clear oil: ¹H-NMR (400 MHz, CDCl₃) δ=7.44-7.33 (m, 5H), 6.85 (m, 4H), 5.01 (s, 2H), 3.78 (s, 3H), 1.55 (s, 6H). MS calculated for C₁₈H₂₁O₄ (M+H⁺) 301.1, found 301.4.

Step B: 2-(4-Benzyloxy-phenoxy)-2-methyl-propionic acid methyl ester 27 (0.5 g, 1.7 mmol) is dissolved in EtOH (15 mL). After addition of a catalytic amount of palladium(0) on charcoal the mixture is subjected to 1 atm hydrogen and stirred for 5 h at rt. Then the mixture is filtered through Celite 545, the solvent is removed and the remainder dried on high vacuum to yield 2-(4-hydroxy-phenoxy)-2-methyl-propionic acid methyl ester 28 as a brown oil: ¹H-NMR (400 MHz, CDCl₃) δ=6.76 (d, J=9.0 Hz, 2H), 6.69 (d, J=9.0 Hz, 2H), 3.78 (s, 3H), 1.53 (s, 6H). MS calculated for C₁₁H₁₅O₄ (M+H⁺) 211.1, found 211.3.

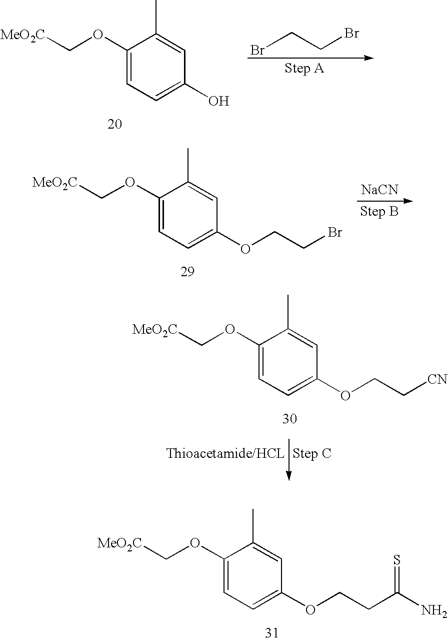

### Intermediate 31

### [2-Methyl-4-(2-thiocarbamoyl-ethoxy)-phenoxy]-acetic Acid Methyl Ester

Step A: (4-Hydroxy-2-methyl-phenoxy)-acetic acid methyl ester 20 (3.0 g, 15.3 mmol) and 1,2-dibromoethane (13.2 mL, 153 mmol) are dissolved in dry acetone (100 mL). Cs₂CO₃ (25 g, 76.5 mmol) is added and the mixture is heated at reflux for 16 h. The mixture is cooled, filtered, concentrated and purified by flash chromatography (EtOAc/Hexanes gradient) to afford [4-(2-bromo-ethoxy)-2-methyl-phenoxy]-acetic acid methyl ester 29 as a white powder: ¹H-NMR (400 MHz, CDCl₃) δ=6.70 (s, 1H), 6.59 (m, 2H), 4.53 (s, 2H), 4.15 (t, J=6.4 Hz, 2H), 3.73 (s, 3H), 3.53 (t, J=6.4 Hz, 2H), 2.20 (s, 3H). MS calcd. for C₁₂H₁₆BrO₄ (M+H⁺) 303.0, found 303.0

Step B: [4-(2-Bromo-ethoxy)-2-methyl-phenoxy]-acetic acid methyl ester 29 (100 mg, 0.33 mmol), and NaCN (16 mg, 0.33 mmol) are dissolved in DMSO (3 mL) and stirred at rt for 6 h. The mixture is diluted with water (10 mL) and extracted with EtOAc (3×10 mL). The organic layers are combined, washed with water (20 mL) and brine (20 mL), dried (MgSO₄), filtered, and concentrated to give crude [4-(2-cyano-ethoxy)-2-methyl-phenoxy]-acetic acid methyl ester 30: ¹H-NMR (400 MHz, CDCl₃) δ=6.89 (s, 1H), 6.58 (s, 2H), 4.53 (s, 2H), 4.06 (t, J=6.4 Hz, 2H), 3.72 (s, 3H), 2.71 (t, J=6.4 Hz, 2H), 2.20 (s, 3H). MS calcd. for C₁₃H₁₅NO₄ (M+H⁺) 250.1, found 250.1.

Step C: [4-(2-Cyano-ethoxy)-2-methyl-phenoxy]-acetic acid methyl ester 30 (236 mg, 0.95 mmol) and thioacetamide (178 mg, 2.37 mmol) are dissolved in dimethylformamide (0.8 mL). HCl (0.9 mL of 4N in dioxane, 3.61 mmol) is added and the mixture is heated at 100° C. for 2 h. The mixture is cooled and poured into a saturated solution of NaHCO₃ (50 mL) and extracted with EtOAc (50 mL). The organic layer is washed with water, then brine, dried (MgSO₄), filtered and evaporated to give [2-methyl-4-(2-thiocarbamoyl-ethoxy)-phenoxy]-acetic acid methyl ester 31 as a red oil. MS calcd. for C₁₃H₁₈NO₄S (M+H⁺) 284.1, found 284.1.

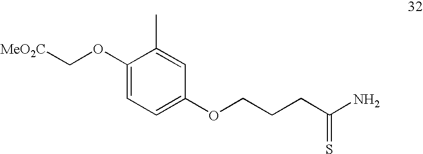

### Intermediate 32

### [2-Methyl-4-(3-thiocarbamoyl-propoxy)-phenoxy]-acetic Acid Methyl Ester

Following the procedure of Intermediate 31, except substituting 1,3-dibromopropane for 1,2-dibromoethane, the title compound is prepared a red oil: MS calcd. for C₁₄H₂₀NO₄S (M+H⁺) 298.1, found 298.1.

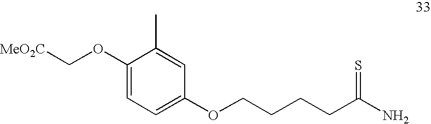

### Intermediate 33

### [2-Methyl-4-(3-thiocarbamoyl-propoxy)-phenoxy]-acetic Acid Methyl Ester

Following the procedure of Intermediate 31, except substituting 1,4-dibromobutane for 1,2-dibromoethane, the title compound is prepared a red oil: ¹H-NMR (400 MHz, CDCl₃) δ=6.65 (s, 1H), 6.56 (m, 2H), 4.52 (s, 2H), 3.86 (t, J=6.4 Hz, 2H), 3.72 (s, 3H), 2.67 (t, J=7.2 Hz, 1H), 2.19 (s, 3H), 1.88 (m, 2H), 1.75 (m, 3H). MS calcd. for C₁₅H₂₂NO₄S (M+H⁺) 312.1, found 312.1.

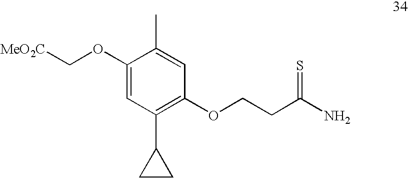

### Intermediate 34

### [5-Cyclopropyl-2-methyl-4-(2-thiocarbamoyl-ethoxy)-phenoxy]-acetic Acid Methyl Ester

Following the procedure of Intermediate 31, except substituting (5-cyclopropyl-4-hydroxy-2-methyl-phenoxy)-acetic acid methyl ester 24 for (4-Hydroxy-2-methyl-phenoxy)-acetic acid methyl ester 20, the title compound is prepared a red oil. MS calcd. for C₁₆H₂₂NO₄S (M+H⁺) 324.1, found 324.1.

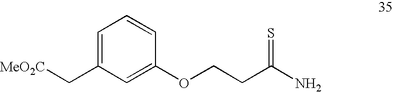

### Intermediate 35

### [3-(2-Thiocarbamoyl-ethoxy)-phenyl]-acetic Acid Methyl Ester

Following the procedure of Intermediate 31, except substituting (3-hydroxy-phenyl)-acetic acid methyl ester 11 for (4-Hydroxy-2-methyl-phenoxy)-acetic acid methyl ester 20, the title compound is prepared a red oil: H-NMR (400 MHz, CDCl₃) δ=7.12 (t, J=8.8 Hz, 1H), 6.76 (m, 3H), 4.13 (t, J=6.4 Hz, 2H), 3.63 (s, 3H), 3.53 (m, 3H), 2.76 (t, J=6.4 Hz, 1H). MS calcd. for C₁₂H₁₆NO₃S (M+H⁺) 254.1, found 254.1.

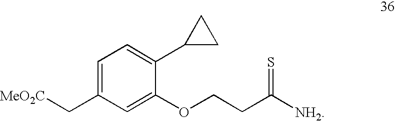

### Intermediate 36

### [4-Cyclopropyl-3-(2-thiocarbamoyl-ethoxy)-phenyl]-acetic Acid Methyl Ester

Following the procedure of Intermediate 31, except substituting (4-Cyclopropyl-3-hydroxy-phenyl)-acetic acid methyl ester 16 for (4-Hydroxy-2-methyl-phenoxy)-acetic acid methyl ester 20, the title compound is prepared a red oil. MS calcd. for C₁₅H₂₀NO₃S (M+H⁺) 294.1, found 294.1.

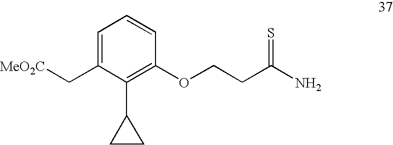

### Intermediate 37

### [2-Cyclopropyl-3-(2-thiocarbamoyl-ethoxy)-phenyl]-acetic Acid Methyl Ester

Following the procedure of Intermediate 31, except substituting (2-Cyclopropyl-3-hydroxy-phenyl)-acetic acid methyl ester 17 for (4-Hydroxy-2-methyl-phenoxy)-acetic acid methyl ester 20, the title compound is prepared a red oil. MS calcd. for C₁₅H₂₀NO₃S (M+H⁺) 294.1, found 294.1.

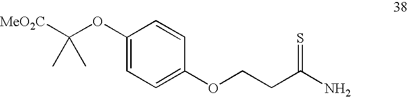

### Intermediate 38

### 2-Methyl-2-[4-(2-thiocarbamoyl-ethoxy)-phenoxy]-propionic Acid Methyl Ester

Following the procedure of Intermediate 31, except substituting 2-(4-Hydroxy-phenoxy)-2-methyl-propionic acid methyl ester 28 for (4-Hydroxy-2-methyl-phenoxy)-acetic acid methyl ester 20, the title compound is prepared a red oil. MS calcd. for C₁₄H₂₀NO₄S (M+H⁺) 298.1, found 298.1.

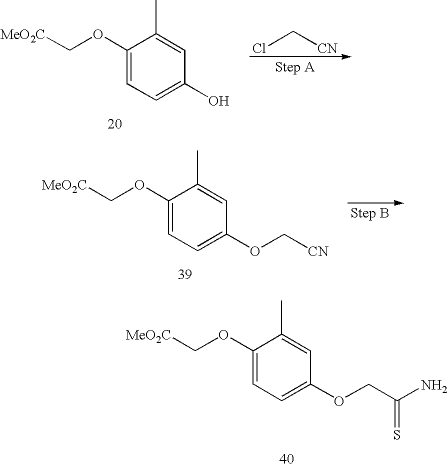

### Intermediate 40

### (2-Methyl-4-thiocarbamoylmethoxy-phenoxy)-acetic Acid Methyl Ester

Step A: (4-Hydroxy-2-methyl-phenoxy)-acetic acid methyl ester 20 (500 mg, 2.55 mmol) and chloroacetonitrile (0.16 mL, 2.55 mmol) are dissolved in dry acetonitrile (10 mL). Cesium carbonate (1.6 g, 5.1 mmol) is added and the mixture is stirred at rt for 16 h. The mixture is filtered and concentrated to afford (4-cyanomethoxy-2-methyl-phenoxy)-acetic acid methyl ester 39 as a colorless glass. ¹H-NMR (400 MHz, CDCl₃) 6.76 (s, 1H), 6.67 (d, J=8.4 Hz, 1H), 6.60 (d, J=8.4 Hz, 1H), 4.62 (s, 2H), 4.54 (s, 2H), 3.72 (s, 3H), 2.21 (s, 3H). MS calcd. for C₁₂H₁₄NO₄ (M+H⁺) 236.1, found 236.1.

Step B: (4-Cyanomethoxy-2-methyl-phenoxy)-acetic acid methyl ester 39 (5.85 g, 24.8 mmol) and thioacetamide (4.76 g, 63.3 mmol) are dissolved in dimethylformamide (15 mL). HCl (24 mL of 4N in dioxane, 95 mmol) is added and the mixture is heated at 100° C. for 2 h. The mixture is cooled and poured into a saturated solution of NaHCO₃ (20 mL) and extracted with EtOAc (50 mL). The organic layer is washed with water, then brine, dried (MgSO₄), filtered and concentrated. The residue is triturated with dichloromethane and filtered to give (2-Methyl-4-thiocarbamoylmethoxy-phenoxy)-acetic acid methyl ester 40 as a beige powder: ¹H-NMR (400 MHz, DMSO-d6) 6.84 (d, J=2.8 Hz, 1H), 6.78 (d, J=8.8 Hz, 1H), 6.71 (dd, J=8.8, 2.8 Hz, 1H), 4.73 (s, 2H), 4.67 (s, 2H), 3.70 (s, 3H), 2.18 (s, 3H). MS calcd. for C₁₂H₁₅NO₄S (M+H⁺) 270.1, found 270.3.

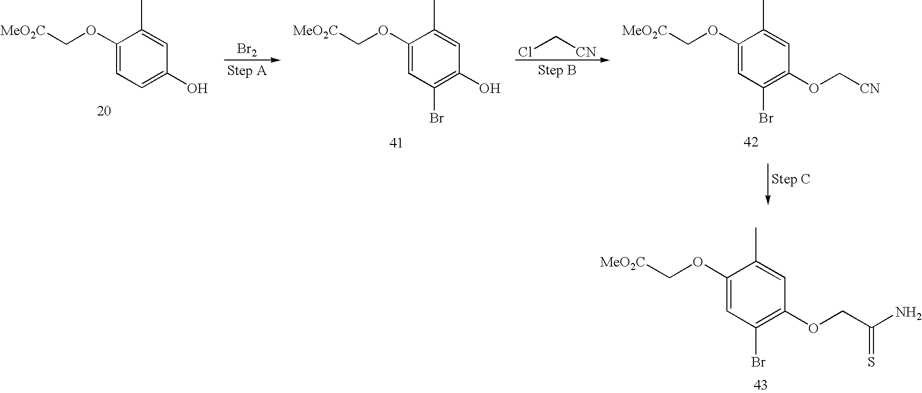

### Intermediate 43

### (5-Bromo-2-methyl-4-thiocarbamoylmethoxy-phenoxy)-acetic Acid Methyl Ester

Step A: (4-Hydroxy-2-methyl-phenoxy)-acetic acid methyl ester 20 (5 g, 25.5 mmol) is dissolved in dichloromethane (100 mL). Bromine (1.44 mL, 28.0 mmol) in dichloromethane (20 mL) is added and the mixture is stirred at rt for 3 h. Then the mixture is washed with water, saturated solutions of NaHCO₃ and sodium bisulfite, dried (MgSO₄) filtered and concentrated. The residue is purified by flash chromatography (EtOAc/Hexanes gradient) to afford the (5-bromo-4-hydroxy-2-methyl-phenoxy)-acetic acid methyl ester 41. ¹H-NMR (400 MHz, CDCl₃) 7.01 (s, 1H), 6.98 (s, 1H), 5.32 (br. s, 1H), 4.74 (s, 2H), 3.98 (s, 3H), 2.39 (s, 3H). MS calcd. for C₁₀H₁₂BrO₄ (M+H⁺) 275.0, found 297.0 (+Na⁺).

Stop B: (5-Bromo-4-hydroxy-2-methyl-phenoxy)-acetic acid methyl ester 41 (3.0 g, 9.31 mmol) and chloroacetonitrile (0.62 mL, 9.78 mmol) are dissolved in dry acetonitrile (50 mL). Cesium carbonate (6.07 g, 18.62 mmol) is added and the mixture is stirred at rt for 2 h. The mixture is filtered and concentrated to afford crude (5-bromo-4-cyanomethoxy-2-methylphenoxy)-acetic acid methyl ester 42 which is used in the next step without further purification. MS calcd. for C₁₂H₁₃BrNO₄ (M+H⁺) 314.0, found 313.9.

Step C: (5-Bromo-4-cyanomethoxy-2-methyl-phenoxy)-acetic acid methyl ester 42 (9.3 mmol) and thioacetamide (1.75 g, 23.2 mmol) are dissolved in dimethylformamide (7 mL). HCl (9 mL of 4N in dioxane, 35.3 mmol) is added and the mixture is heated at 100° C. for 2 h. The mixture is cooled and poured into a saturated solution of NaHCO₃ (20 mL) and extracted with EtOAc (20 mL). The organic layer is washed with water, then brine, dried (MgSO₄), filtered and concentrated. The residue is recrystallized from dichloromethane/hexanes to give (5-Bromo-2-methyl-4-thiocarbamoylmethoxy-phenoxy)-acetic acid methyl ester 43 as a beige powder. ¹H-NMR (400 MHz, CDCl₃) 8.27 (s, 1H), 7.68 (s, 1H), 6.91 (s, 1H), 6.76 (s, 1H), 4.85 (s, 2H), 4.61 (s, 2H), 3.81 (s, 3H), 2.25 (s, 3H). MS calcd. for C₁₂H₁₅BrNO₄S (M+H⁺) 348.0, found 347.9.

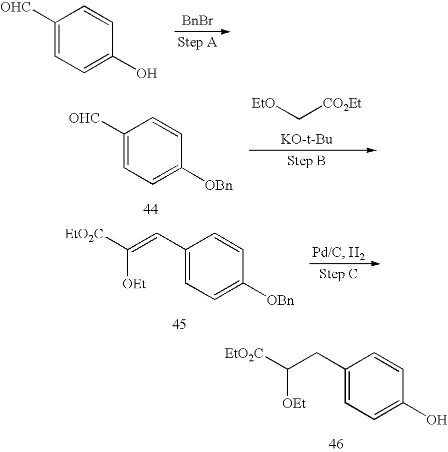

### Intermediate 46

### (±)-2-Ethoxy-3-(4-hydroxy-phenyl)-propionic Acid Ethyl Ester

Step A: 4-Hydroxybenzaldehyde (7.03 g, 57.6 mmol) is dissolved in acetonitrile (60 mL). Powdered potassium carbonate (11.98 g, 86.7 mmol) is added while stirring, followed by dropwise addition of the benzyl bromide (7 mL, 59 mmol). The mixture is vigorously stirred under nitrogen for 3 h. Filtration and concentration yielded 4-benzyloxybenzaldehyde 44 (12.4 g, quant.) as a white solid: ¹H-NMR (400 MHz, CDCl₃) δ=9.89 (s, 1H), 7.84 (d, J=8.8 Hz, 2H), 7.40 (m, 5H), 7.08 (d, J=8.8 Hz, 2H), 5.16 (s, 2H). MS calcd. for C₁₄H₁₃O₂ (M+H⁺) 213.1, found 213.2.

Step B: 4-Benzyloxy-benzaldehyde 44 (1.24 g, 5.84 mmol) and ethyl ethoxyacetate (1.2 mL, 8.8 mmol) are dissolved in dry THF (30 mL). Solid potassium tert-butoxide (1.45 g, 12.9 mmol) is added and the mixture is stirred under nitrogen overnight. The resulting suspension is filtered through Celite 545. The solids are thoroughly washed with THF. The combined organic solutions are concentrated to yield 3-(4-benzyloxy-phenyl)-2-ethoxy-acrylic acid ethyl ester 45 as an oil. The crude material is used as such in the next step: ¹H-NMR (400 MHz, CDCl₃) δ=7.75 (d, J=8.8 Hz, 2H), 7.37 (m, 5H), 6.96 (d, J=8.8 Hz, 2H), 6.95 (s, 1H), 5.09 (s, 2H), 4.29 (q, J=7.2 Hz, 2H), 3.98 (q, J=7.1 Hz, 2H), 1.35 (m, 6H). MS calcd. for C₂₀H₂₃O₄ (M+H⁺) 327.2, found 327.2.

Step C: 3-(4-Benzyloxy-phenyl)-2-ethoxy-acrylic acid ethyl ester 45 (0.80 g, 2.45 mmol) is dissolved in ethanol (40 mL). The solution is degassed with nitrogen, then treated with a catalytic amount of 5% palladium black on carbon (0.28 g, 0.13 mmol). The solution is shaken under 60 psi hydrogen for 5 h. Filtration and concentration yielded (±)-2-ethoxy-3-(4-hydroxy-phenyl)-propionic acid ethyl ester 46 as an oil. ¹H-NMR (400 MHz, CDCl₃) δ=7.10 (d, J=8.4 Hz, 2H), 6.74 (d, J=8.4 Hz, 2H), 4.18 (q, J=7.2 Hz, 2H), 3.97 (t, J=6.9 Hz, 1H), 3.60 (m, 1H), 3.36 (m, 1H), 2.94 (d, J=6.6 Hz, 2H), 1.23 (t, J=7.2 Hz, 3H), 1.17 (t, J=7.0 Hz, 3H). MS calcd. for C₁₃H₁₉O₄ (M+H⁺) 239.1, found 239.1.

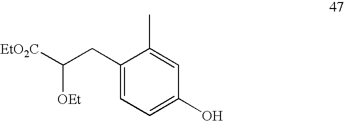

### Intermediate 47

### (±)-2-Ethoxy-3-(4-hydroxy-2-methyl-phenyl)-propionic Acid Ethyl Ester

Following the procedure for Intermediate 46, except substituting 4-hydroxy-2-methylbenzaldehyde for 4-hydroxybenzaldehyde, the title compound is prepared as a clear oil: MS calcd. for C₁₄H₂₀NaO₄ (M+Na⁺) 275.1, found 275.1.

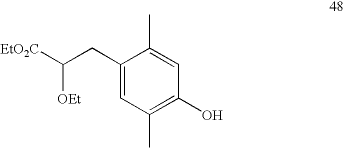

### Intermediate 48

### (±)-2-Ethoxy-3-(4-hydroxy-2,5-dimethylphenyl)-propionic Acid Ethyl Ester

Following the procedure for Intermediate 46, except substituting 4-hydroxy-2,5-dimethylbenzaldehyde for 4-hydroxybenzaldehyde, the title compound is prepared as a clear oil: MS calcd. for C₁₄H₂₀NaO₄ (M+Na⁺) 275.1, found 275.2.

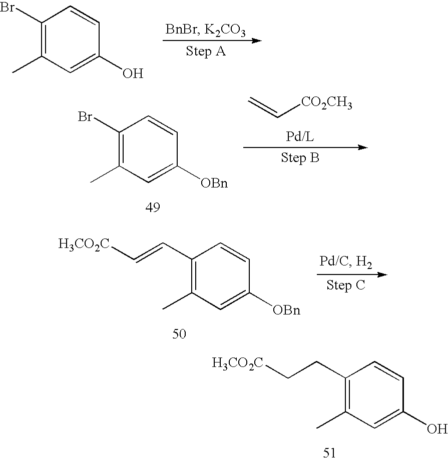

### Intermediate 51

### 3-(4-Hydroxy-2-methyl-phenyl)-propionic Acid Methyl Ester

Step A: 4-Bromo-3-methyl-phenol (25.11 g, 134 mmol) is dissolved in acetonitrile (125 mL). Powdered potassium carbonate (25.69 g, 186 mmol) is added while stirring, followed by dropwise addition of benzyl bromide (17 mL, 143 mmol). The mixture is vigorously stirred under nitrogen for 6 h. Filtration through a plug of Celite 545 and concentration yielded 4-benzyloxy-1-bromo-2-methyl-benzene 49 as an off-white solid: ¹H-NMR (400 MHz, CDCl₃) δ=7.35 (m, 6H), 6.87 (d, J=2.8 Hz, 1H), 6.68 (dd, J=8.8, 2.8 Hz, 1H), 5.02 (s, 2H), 2.36 (s, 3H).

Step B: 4-Benzyloxy-1-bromo-2-methyl-benzene 49 (24.0 g, 86.6 mmol), tri-o-tolyl-phosphane (15.00 g, 49.3 mmol), ethyl diisopropylamine (35 mL, 212 mmol) and methyl acrylate (35 mL, 388 mmol) are dissolved in propionitrile (200 mL). The mixture is degassed with argon. Solid palladium(II) acetate (4.00 g, 17.8 mmol) is added and the mixture is heated to 100° C. for 18 h. The mixture is cooled and filtered through a plug of Celite 545. Concentration and silica gel purification (0-40% gradient of ethyl acetate in hexanes) yielded 3-(4-benzyloxy-2-methyl-phenyl)-acrylic acid ethyl ester 50 as an oil (30.8 g, quant.): ¹H-NMR (400 MHz, CDCl₃) δ=7.92 (d, J=15.8 Hz, 1H), 7.52 (d, J=9.4 Hz, 1H), 7.39 (m, 5H), 6.82 (m, 2H), 6.26 (d, J=15.8 Hz, 1H), 5.08 (s, 2H), 3.80 (s, 3H), 2.42 (s, 3H). MS calcd. for C₁₈H₁₉O₃ (M+H⁺) 283.2, found 283.2.

Step C: 3-(4-Benzyloxy-2-methyl-phenyl)-acrylic acid ethyl ester 50 from Step B above is dissolved in ethyl acetate (200 mL) and ethanol (20 mL). The solution is degassed with nitrogen, then treated with 5% palladium black on carbon (1.15 g, 1.08 mmol, 1 mol %). The solution is shaken under 40 psi hydrogen for 15 h. Filtration and concentration yielded 3-(4-hydroxy-2-methyl-phenyl)-propionic acid methyl ester 51 as an oil: ¹H-NMR (400 MHz, CDCl₃) δ=6.98 (d, J=8.1 Hz, 1H), 6.64 (d, J=2.6 Hz, 1H), 6.60 (dd, J=8.1, 2.6 Hz, 1H), 4.93 (m, 1H), 3.68 (s, 1H), 2.86 (t, J=8.8 Hz, 2H), 2.55 (d, J=8.8 Hz, 2H), 2.26 (s, 3H) MS calcd. for C₁₁H₁₄NaO₃ (M+Na⁺) 217.1, found 217.1.

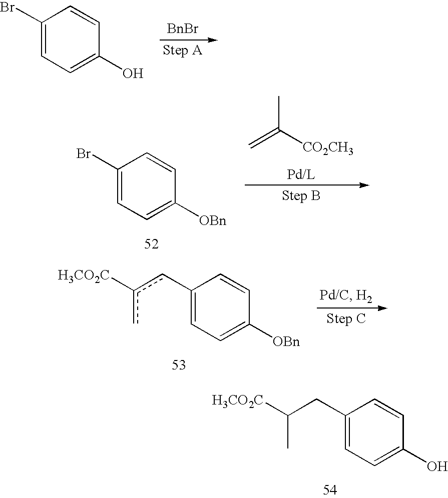

### Intermediate 54

### (±)-3-(4-Hydroxy-phenyl)-2-methyl-propionic Acid Methyl Ester

Step A: 4-Bromophenol (3.55 g, 20.5 mmol) is dissolved in acetonitrile (50 mL). Powdered potassium carbonate (3.86 g, 27.9 mmol) is added while stirring, followed by dropwise addition of benzyl bromide (2.4 mL, 20.2 mmol). The mixture is vigorously stirred under nitrogen for 6 h. Filtration and concentration yielded 4-benzyloxy-bromobenzene 52 (5.52 g, quant.) as an oil that slowly solidified: ¹H-NMR (400 MHz, CDCl₃) δ=7.37, (m, 7H), 6.87 (m, 2H), 5.07 (s, 2H).

Step B: 4-Benzyloxy-bromobenzene 52 (1.30 g, 5.2 mmol), tri-o-tolyl-phosphane (0.98 g, 3.2 mmol), ethyl diisopropylamine (2 mL, 12.1 mmol) and methyl methacrylate (2.20 mL, 20.7 mmol) are dissolved in propionitrile (100 mL). The mixture is degassed with argon. Solid palladium(II) acetate (0.26 g, 1.2 mmol) is added and the mixture is heated to 100° C. for 18 h. The mixture is cooled and filtered through a plug of Celite 545. Concentration and silica gel purification (10-60% gradient of ethyl acetate in hexanes) yielded a 1:1 mixture of isomeric olefins 53 as an oil. Used the mixture as such in the next step: MS calcd. for C₁₈H₁₉O₃ (M+H⁺) 283.1, found 283.1.

Step C: The 1:1 olefin mixture 53 from Step B above is dissolved in ethyl acetate (50 mL) and ethanol (10 mL). The solution is degassed with nitrogen, then treated with a catalytic amount of 5% palladium black on carbon (0.50 g, 7 mol %). The solution is shaken under 60 psi hydrogen for 15 h. Filtration and concentration yielded (±)-3-(4-hydroxyphenyl)-2-methyl-propionic acid methyl ester 54 as an oil: ¹H-NMR (400 MHz, CDCl₃) δ=7.01 (d, J=8.6 Hz, 2H), 6.74 (d, J=8.6 Hz, 2H), 3.63 (s, 3H), 2.94 (dd, J=13.1, 6.7 Hz, 1H), 2.69 (dqd, J=7.4, 6.8, 6.7 Hz, 1H), 2.60 (dd, J=13.1, 7.4 Hz, 1H), 1.14 (d, J=6.8 Hz, 3H). MS calcd. for C₁₁H₁₄NaO₃ (M+Na⁺) 217.1, found 217.1.

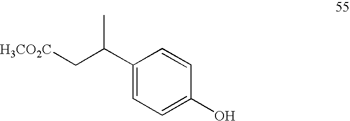

### Intermediate 55

### (±)-3-(4-Hydroxy-phenyl)-butyric Acid Methyl Ester

Following the procedure for Intermediate 54, except substituting methyl crotonate for methyl methacrylate in Step B, the title compound is prepared as a clear liquid: ¹H-NMR (400 MHz, CDCl₃) δ=7.08 (d, J=8.9 Hz, 2H), 6.75 (d, J=8.6 Hz, 2H), 4.91 (s, 1H), 3.62 (s, 3H), 3.22 (m, 1H), 2.55 (m, 2H), 1.27 (d, J=7.0 Hz, 3H). MS calcd. for C₁₁H₁₄NaO₃ (M+Na⁺) 217.1, found 217.1.

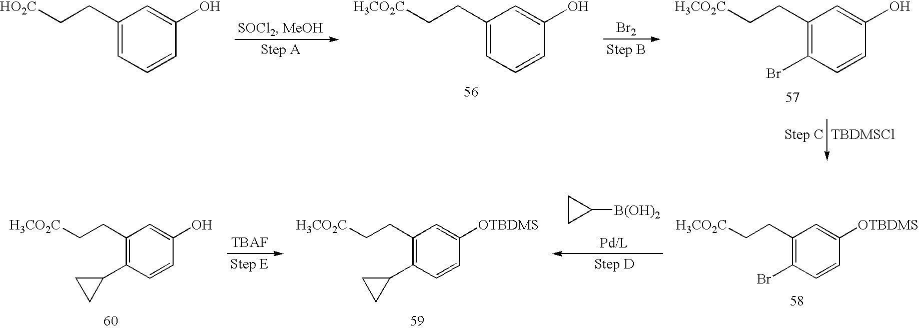

### Intermediate 60

### 3-(2-Cyclopropyl-5-hydroxy-phenyl)-propionic Acid Methyl Ester

Step A: 3-(3-Hydroxy-phenyl)-propionic acid (24.88 g, 149.7 mmol) is dissolved in methanol (50 mL). Thionyl chloride (5 mL, 68.7 mmol) is added dropwise with vigorous stirring. The mixture is stirred at 60° C. for 3 h. Cooling and concentration yielded 3-(3-hydroxy-phenyl)-propionic acid methyl ester 56 (29.26 g, quant.) as an oil: ¹H-NMR (400 MHz, CDCl₃) δ=7.15 (dd, J=8.4, 7.6 Hz, 1H), 6.75 (d, J=7.6 Hz, 1H), 6.69 (m, 2H), 3.68 (s, 3H), 2.90 (t, J=7.6 Hz, 2H), 2.63 (t, J=7.6 Hz, 2H).

Step B: 3-(3-Hydroxy-phenyl)-propionic acid methyl ester 56 (3.16 g, 17.5 mmol) is dissolved in DCM (40 mL). Powdered calcium carbonate (2.27 g, 22.7 mmol) is added. While the suspension is vigorously stirred, a solution of bromine (0.90 mL, 17.6 mmol) in DCM (30 mL) is added dropwise. After the addition is completed, the suspension is treated with 0.2 g sodium bisulfite in water (5 mL). The organic layer is dried over MgSO₄, filtered and concentrated to yield 3-(2-bromo-5-hydroxy-phenyl)-propionic acid methyl ester 57 as a colourless oil. ¹H-NMR (400 MHz, CDCl₃) δ=7.36 (d, J=8.6 Hz, 1H), 6.76 (d, J=3.0 Hz, 1H), 6.60 (dd, J=8.6, 3.0 Hz, 1H), 5.46 (s, 1H), 3.69 (s, 3H), 3.00 (t, J=7.9 Hz, 2H), 2.65 (t, J=7.9 Hz, 2H).

Step C: 3-(2-Bromo-5-hydroxy-phenyl)-propionic acid methyl ester 57 (4.45 g, 17.2 mmol) is dissolved in DCM (80 mL). Imidazole (1.45 g, 21.3 mmol) is added and the mixture is stirred at rt until it became homogenous. tert-Butyl dimethylchlorosilane (2.66 g, 17.7 mmol) is added and the mixture is stirred at rt for 18 h. Washing with water, drying over MgSO₄ and concentration yielded 3-[2-bromo-5-(tert-butyldimethyl-silanyloxy)-phenyl]-propionic acid methyl ester 58 as an oil: ¹H-NMR (400 MHz, CDCl₃) (two major rotamers are present; the data is given for the most abundant isomer) δ=7.35 (d, J=8.6 Hz, 1H), 6.74 (d, J=2.9 Hz, 1H), 6.58 (dd, J=8.6, 2.9 Hz, 1H), 3.69 (s, 3H), 2.99 (t, J=8.2 Hz, 2H), 2.63 (t, J=8.2 Hz, 2H), 0.97 (s, 9H), 0.18 (s, 6H). MS calcd. for C₁₆H₂₆BrO₃Si (M+H⁺) 373.1, found 373.1.

Step D: 3-[(2-Bromo-5-(tert-butyl-dimethyl-silanyloxy)-phenyl]-propionic acid methyl ester 58 (5.74 g, 15.4 mmol) is dissolved in toluene (165 mL). Cyclopropylboronic acid (2.22 g, 25.8 mmol), potassium phosphate (11.71 g, 55.2 mmol), and tricyclohexyl-phosphane (1.81 g, 6.5 mmol) are added, followed by water (10 mL). The mixture is degassed with argon. Palladium(II) acetate (0.70 g, 3.1 mmol) is added. The mixture is heated to 95° C. for 3.5 h. Cooling, separation of the organic layer, drying over MgSO₄ and concentration, followed by silica gel chromatography (0-25% gradient, ethyl acetate in hexanes) yielded 3-[5-(tert-butyl-dimethyl-silanyloxy)-2-cyclopropyl-phenyl]-propionic acid methyl ester 59 as an oil: ¹H-NMR (400 MHz, CDCl₃) δ=6.84 (d, J=8.3 Hz, 1H), 6.63 (d, J=2.5 Hz, 1H), 6.59 (dd, J=8.3, 2.5 Hz, 1H), 3.70 (s, 3H), 3.08 (t, J=7.8 Hz, 2H), 2.64 (t, J=7.8 Hz, 2H), 1.83 (m, 1H), 0.97 (s, 9H), 0.88 (m, 2H), 0.58 (m, 2H), 0.16 (s, 6H). MS calcd. for C₁₉H₃₁O₃Si (M+H⁺) 335.2, found 335.2.

Step E: 3-[5-(tert-Butyl-dimethyl-silanyloxy)-2-cyclopropyl-phenyl]-propionic acid methyl ester 59 (2.87 g, 8.6 mmol) is dissolved in THF (30 mL). A 1 M solution of tetra-(n-butyl)ammonium fluoride in THF (10 mL, 10 mmol) is added. The mixture is stirred at rt for 4 h. Concentration to dryness and purification by silica gel chromatography (10-60% gradient, ethyl acetate in hexanes) yielded 3-(2-cyclopropyl-5-hydroxy-phenyl)-propionic acid methyl ester 60: ¹H-NMR (400 MHz, CDCl₃) δ=6.87 (d, J=8.3 Hz, 1H), 6.65 (d, J=2.7 Hz, 1H), 6.60 (dd, J=8.3, 2.7 Hz, 1H), 4.96 (s, 1H), 3.70 (s, 3H), 3.09 (t, J=7.8 Hz, 2H), 2.65 (t, J=7.8 Hz, 2H), 1.82 (m, 1H), 0.88 (m, 2H), 0.58 (m, 2H). MS calcd. for C₁₃H₁₇O₃ (M+H⁺) 221.1, found 221.1.

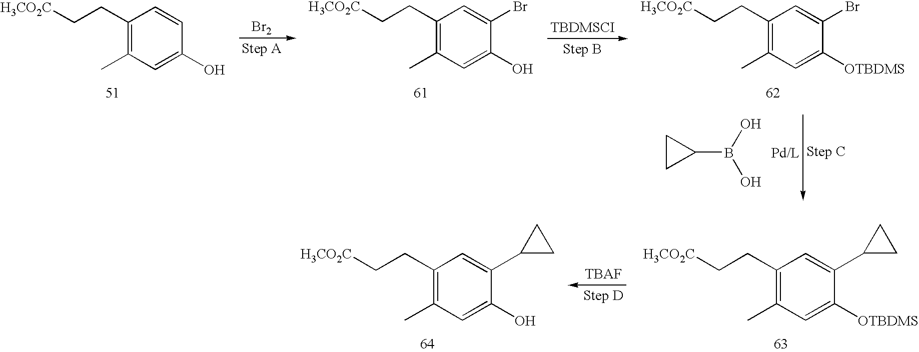

### Intermediate 64

### 3-(5-Cyclopropyl-4-hydroxy-2-methyl-phenyl)-propionic Acid Methyl Ester

Step A: 3-(4-Hydroxy-2-methyl-phenyl)-propionic acid methyl ester 51 (2.50 g, 12.9 mmol) is dissolved in DCM (60 mL) and cooled to 0° C. Powdered calcium carbonate (2.27 g, 22.7 mmol) is added. While the suspension is vigorously stirred, a solution of bromine (0.90 mL, 17.6 mmol) in DCM (20 mL) is added dropwise. After the addition is completed, the suspension is warmed up to rt and treated with 0.2 g sodium bisulfite and 5 mL water, followed by drying over MgSO₄, filtration and concentration to yield 3-(5-bromo-4-hydroxy-2-methyl-phenyl)-propionic acid methyl ester 61 (3.67 g, quant.) as a colourless oil that slowly solidified. ¹H-NMR (400 MHz, CDCl₃) δ=7.21 (s, 1H), 6.82 (s, 1H), 5.30 (s, 1H), 3.69 (s, 3H), 2.85 (t, J=7.5 Hz, 2H), 2.54 (t, J=7.5 Hz, 2H), 2.24 (s, 3H).

Step B: 3-(5-Bromo-4-hydroxy-2-methyl-phenyl)-propionic acid methyl ester 61 (from Step A above) is dissolved in DCM (45 mL). Imidazole (1.12 g, 16.5 mmol) is added and the mixture is stirred at rt until it became homogenous. tert-Butyl dimethylchlorosilane (2.10 g, 13.9 mmol) is added and the mixture is stirred at rt for 18 h. Washing with water, drying the organic phase over MgSO₄ and concentration yielded 3-[5-bromo-4-(tert-butyl-dimethyl-silanyloxy)-2-methyl-phenyl]propionic acid methyl ester 62 as an oil: ¹H-NMR (400 MHz, CDCl₃) δ=7.25 (s, 1H), 6.65 (s, 1H), 3.68 (s, 3H), 2.83 (t, J=7.6 Hz, 2H), 2.54 (t, J=7.6 Hz, 2H), 2.21 (s, 3H), 1.03 (s, 9H), 0.23 (s, 6H).

Step C: 3-[5-Bromo-4-(tert-butyl-dimethyl-silanyloxy)-2-methyl-phenyl]-propionic acid methyl ester 62 (4.67 g, 12.1 mmol) is dissolved in toluene (70 mL). Cyclopropylboronic acid (1.95 g, 22.7 mmol), potassium phosphate (9.15 g, 43.1 mmol), and tricyclohexyl-phosphane (1.44 g, 5.13 mmol) are added, followed by water (10 mL). The mixture is degassed with argon. Palladium(II) acetate (0.55 g, 2.45 mmol) is added. The mixture is heated to 95° C. for 3.5 h. Cooling, separation of the organic layer, drying over MgSO₄ and concentration, followed by silica gel chromatography (0-20% gradient, ethyl acetate in hexanes) yielded 3-[4-(tert-butyldimethyl-silanyloxy)-5-cyclopropyl-2-methyl-phenyl]-propionic acid methyl ester 63 as an oil: ¹H-NMR (400 MHz, CDCl₃)=6.56 (s, 1H), 6.54 (s, 1H), 3.67 (s, 3H), 2.81 (t, J=7.7 Hz, 2H), 2.51 (t, J=7.7 Hz, 2H), 2.21 (s, 3H), 1.76 (m, 1H), 1.01 (s, 9H), 0.85 (m, 2H), 0.57 (m, 2H), 0.22 (s, 6H). MS calcd. for C₂₀H₃₃O₃Si (M+H⁺) 349.2, found 349.2.

Step D: 3-[4-(tert-Butyl-dimethyl-silanyloxy)-5-cyclopropyl-2-methyl-phenyl]-propionic acid methyl ester 63 (4.23 g, 12.1 mmol) is dissolved in THF (60 mL). A 1 M solution of tetra(n-butyl)ammonium fluoride in THF (18 mL, 18 mmol) is added. The mixture is stirred at rt for 4 h. Concentration to dryness and purification by silica gel chromatography (10-30% gradient, ethyl acetate in hexanes) yielded 3-(5-cyclopropyl-4-hydroxy-2-methyl-phenyl)-propionic acid methyl ester 64: ¹H-NMR (400 MHz, CDCl₃) δ=6.84 (s, 1H), 6.67 (s, 1H), 5.30 (s, 1H), 3.68 (s, 3H), 2.84 (t, J=7.7 Hz, 2H), 2.53 (t, J=7.7 Hz, 2H), 2.24 (s, 3H), 1.74 (m, 1H), 0.93 (m, 2H), 0.60 (m, 2H). MS calcd. for C₁₄H₁₉O₃ (M+H⁺) 235.1, found 235.1.

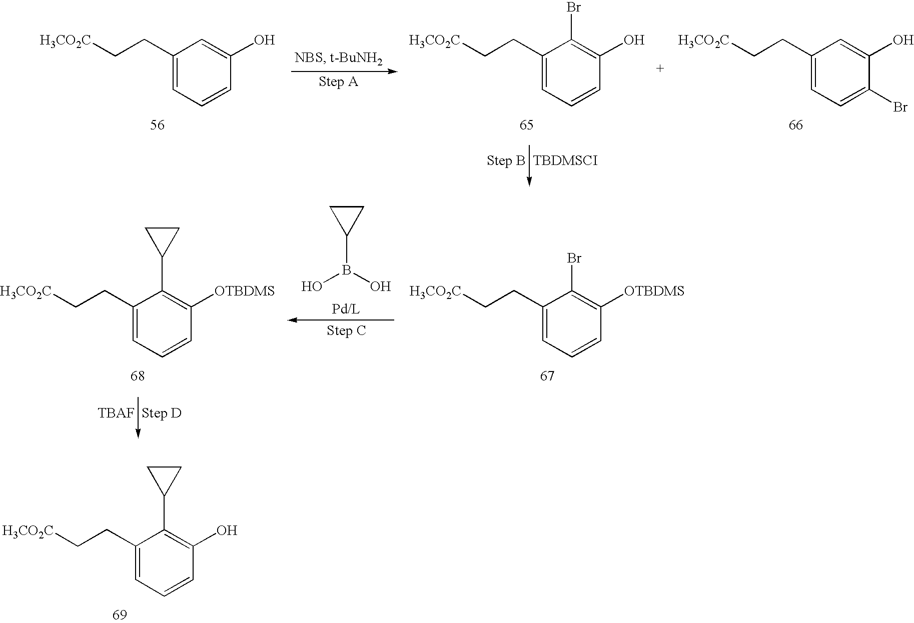

### Intermediate 69

### 3-(2-Cyclopropyl-3-hydroxy-phenyl)-propionic Acid Methyl Ester

Step A: N-Bromosuccinimide (7.56 g, 42.5 mmol) is suspended in DCM (50 mL). tert-Butylamine (5 mL, 47.5 mmol) is added in one portion. After 45 min, the white precipitate is filtered off and the clear filtrate is used as such.

3-(3-Hydroxy-phenyl)-propionic acid methyl ester (7.56 g, 42 mmol) is dissolved in DCM (25 mL) and cooled to −78° C. The clear filtrate prepared above is added dropwise with stirring. After 30 min, the mixture is warmed up and concentrated to yield a mixture of 65 and 66. Trituration with DCM resulted in the precipitation of dibrominated sideproduct and is filtered off. Silica gel chromatography purification (10-100% ethyl acetate in hexanes) of the filtrate yielded 3-(2-bromo-3-hydroxy-phenyl)-propionic acid methyl ester 65 and 3-(4-bromo-3-hydroxy-phenyl)-propionic acid methyl ester 66: 65: ¹H-NMR (400 MHz, CDCl₃) δ=7.14 (t, J=7.8 Hz, 1H), 6.90 (dd, J=8.2, 1.4 Hz, 1H), 6.82 (dd, J=7.4, 1.4 Hz, 1H), 5.68 (s, 1H), 3.69 (s, 3H), 3.07 (t, J=8.0 Hz, 2H), 2.65 (t, J=8.0 Hz, 2H). 66: ¹H-NMR (400 MHz, CDCl₃) δ 7.35 (d, J=8.4 Hz, 1H), 6.87 (d, J=2.0 Hz, 1H) 6.66 (dd, J=8.4, 2.0 Hz, 1H), 5.45 (s, 1H), 3.67 (s, 3H), 2.88 (t, J=7.6 Hz, 2H), 2.61 (t, J=7.6 Hz, 2H).

Step B: 3-(2-Bromo-3-hydroxy-phenyl)-propionic acid methyl ester 65 (0.86 g, 3.32 mmol) is dissolved in DCM (15 mL). Imidazole (0.36 g, 5.3 mmol) is added and the mixture is stirred at rt until homogenous. tert-Butyl dimethylchlorosilane (0.55 g, 3.6 mmol) is added and the mixture is stirred at rt for 18 h. Washing with water, drying over MgSO₄ and concentration yielded 3-[2-bromo-3-(tert-butyl-dimethyl-silanyloxy)-phenyl]-propionic acid methyl ester 67 as an oil: ¹H-NMR (400 MHz, CDCl₃) δ=7.07 (t, J=7.6 Hz, 1H), 6.83 (dd, J=7.6, 1.2 Hz, 1H), 6.28 (dd, J=8.0, 1.6 Hz, 1H), 3.67 (s, 3H), 3.06 (t, J=7.6 Hz, 2H), 2.63 (t, J=7.6 Hz, 2H), 1.03 (s, 9H), 0.34 (s, 6H) MS calcd. for C₁₆H₂₆BrO₃Si (M+H⁺) 373.1, found 372.6.

Step C: 3-[2-Bromo-3-(tert-butyl-dimethyl-silanyloxy)-phenyl]-propionic acid methyl ester 67 (1.18 g, 3.16 mmol) is dissolved in toluene (25 mL). Cyclopropylboronic acid (0.55 g, 6.4 mmol), potassium phosphate (2.60 g, 12.2 mmol), and tricyclohexyl-phosphane (0.38 g, 1.36 mmol) are added, followed by water (5 mL). The mixture is degassed with argon. Palladium(II) acetate (0.16 g, 0.71 mmol) is added and the mixture is heated to 95° C. for 4 h. Cooling, separation of the organic layer, drying over MgSO₄ and concentration, followed by silica gel chromatography (0-30% gradient, ethyl acetate in hexanes) yielded 3-[3-(tert-butyl-dimethyl-silanyloxy)-2-cyclopropyl-phenyl]-propionic acid methyl ester 68 as an oil: ¹H-NMR (400 MHz, CDCl₃) δ=7.01 (t, J=7.8 Hz, 1H), 6.75 (d, J=7.6 Hz, 1H), 6.64 (d, J=8.0, Hz, 1H), 3.68 (s, 3H), 3.17 (t, J=7.9 Hz, 2H), 2.63 (t, J=7.9 Hz, 2H), 1.54 (m, 1H), 1.03 (s, 9H), 0.96 (m, 2H), 0.62 (m, 2H), 0.34 (s, 6H). MS calcd. for C₁₉H₃₁O₃Si (M+H⁺) 335.2, found 335.2.

Step D: 3-[3-(tert-Butyl-dimethyl-silanyloxy)-2-cyclopropyl-phenyl]-propionic acid methyl ester 68 (0.72 g, 2.2 mmol) is dissolved in THF (3 mL). A 1 M solution of tetra-(n-butyl)ammonium fluoride in THF (4 mL, 4 mmol) is added and the mixture is stirred at rt for 18 h. Concentration to dryness and purification by silica gel chromatography (5-50% gradient, ethyl acetate in hexanes) yielded 3-(2-cyclopropyl-3-hydroxyphenyl)-propionic acid methyl ester 69: ¹H-NMR (400 MHz, CDCl₃) δ=7.09 (t, J=7.9 Hz, 1H), 6.76 (dd, J=8.1, 0.9 Hz, 1H), 6.72 (d, J=7.6 Hz, 1H), 5.93 (s, 1H), 3.69 (s, 3H), 3.15 (t, J=7.8 Hz, 2H), 2.63 (t, J=7.8 Hz, 2H), 1.59 (m, 1H), 1.14 (m, 2H), 0.65 (m, 2H) MS calcd. for C₁₃H₁₇O₃ (M+H⁺) 221.1, found 221.1.

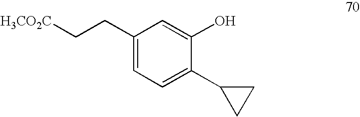

### Intermediate 70

### 3-(4-Cyclopropyl-3-hydroxy-phenyl)-propionic Acid Methyl Ester

Following the procedure for Intermediate 69, except substituting intermediate 66 for intermediate 65 in step B, the title compound is prepared as a clear oil: ¹H-NMR (400 MHz, CDCl₃) δ=6.99 (d, J=7.7 Hz, 1H), 6.71 (d, J=1.5 Hz, 1H), 6.69 (dd, J=7.7, 1.5 Hz, 1H), 5.47 (s, 1H), 3.67 (s, 3H), 2.88 (t, J=7.6 Hz, 2H), 2.61 (t, J=7.6 Hz, 2H), 1.76 (m, 1H), 0.94 (m, 2H), 0.62 (m, 2H). MS calcd. for C₁₃H₁₇O₃ (M+H⁺) 221.1, found 221.1.

with the methyl group at 2.19 ppm). MS calcd. for C₁₃H₁₇O₄ (M+H⁺) 225.1, found 225.1.

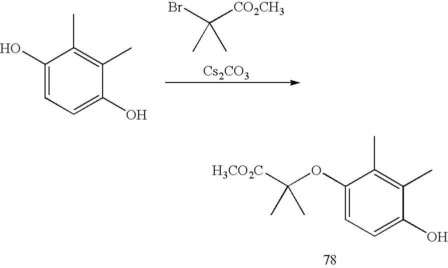

### Intermediate 78

### 2-(4-Hydroxy-2,3-dimethyl-phenoxy)-2-methyl-propionic Acid Methyl Ester

Following the procedure for Intermediate 77, except substituting the appropriate hydroquinone, the title compound is prepared as a clear liquid: ¹H-NMR (400 MHz, CDCl₃) δ=6.50 (s, 2H), 4.55 (s, 1H), 2.05 (s, 3H), 2.04 (s, 3H), 1.52 (s, 6H). MS calcd. for C₁₃H₁₈NaO₄ (M+Na⁺) 261.1, found 261.1.

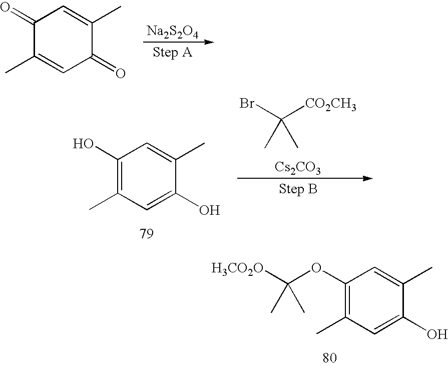

### Intermediate 80

### 2-(4-Hydroxy-2,5-dimethyl-phenoxy)-2-methyl-propionic Acid Methyl Ester

Step A: 2,5-Dimethylquinone (5.41 g, 39.7 mmol) is suspended in diethyl ether (70 mL). Water (100 mL) is added, followed by solid sodium dithionite (20.30 g, 116.6 mmol). The resulting mixture is shaken vigorously. The initially yellow mixture turned deep red, then colourless. Separation of the organic layer, washing with water and brine, drying over Na₂SO₄ and concentration yielded 2,5-dimethylhydroquinone 79 as a white solid: ¹H-NMR (400 MHz, DMSO-d₆) δ=8.32 (s, 2H), 6.45 (s, 2H), 1.99 (s, 6H).

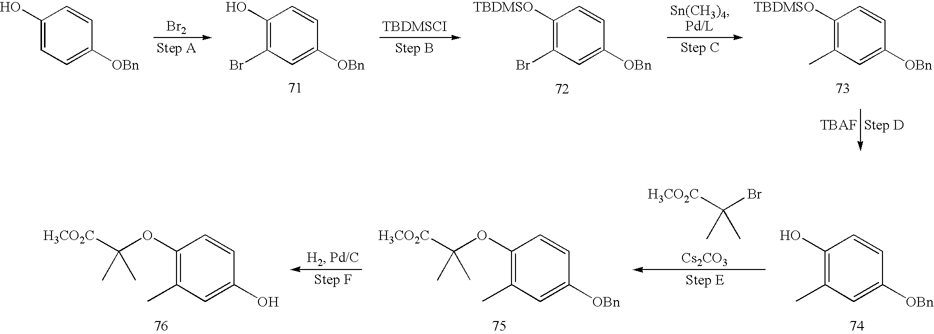

### Intermediate 76

### 2-(4-Hydroxy-2-methyl-phenoxy)-2-methyl-propionic Acid Methyl Ester

Step A: 4-Benzyloxy-phenol (32.04 g, 160 mmol) is dissolved in of DCM (550 mL) and methanol (20 mL). Powdered calcium carbonate (21.83 g, 218 mmol) is suspended into the solution. While stirring vigorously, a solution of bromine (8.30 mL, 162 mmol) in DCM (50 mL) is added dropwise. After the addition is completed, the suspension is stirred at rt for 30 min, then the solids are filtered off. The filtrate is dried over solid NaHCO₃ and MgSO₄, then filtered and concentrated to yield an oil. Recrystallization from diethyl ether/petroleum ether at −20° C. yielded 4-benzyloxy-2-bromo-phenol 71 as a colourless oil that slowly solidified: ¹H-NMR (400 MHz, CDCl₃) δ=7.38 (m, 5H), 7.10 (d, J=2.8 Hz, 1H), 6.94 (d, J=8.9 Hz, 1H), 6.87 (dd, J=8.9, 2.8 Hz, 1H), 4.99 (s, 2H).

Step B: 4-Benzyloxy-2-bromo-phenol 71 (43.6 g, 156 mmol) is dissolved in DCM (400 mL). Imidazole (14.9 g, 219 mmol) is added and the mixture is stirred at rt until homogenous. tert-Butyl dimethylchlorosilane (23.6 g, 156.6 mmol) is added and the mixture is stirred at rt for 18 h. Washing with water, drying over MgSO₄ and concentration yielded (4-benzyloxy-2-bromo-phenoxy)-tert-butyl-dimethyl-silane 72 as an oil: ¹H-NMR (400 MHz, CDCl₃) δ=7.40 (m, 5H), 7.10 (s, 1H), 6.79 (s, 2H), 4.98 (s, 2H), 1.03 (9, 9H), 0.22 (s, 6H).

Step C: (4-Benzyloxy-2-bromo-phenoxy)-tert-butyl-dimethyl-silane 72 (10.05 g, 25.6 mmol) is dissolved in dimethylformamide (45 mL). The mixture is degassed with argon. Dichloro bis(triphenylphosphino)palladium(II) (3.49 g, 4.97 mmol) is added, followed by tetramethyltin (5.0 mL, 36.3 mmol). The mixture is heated to 100° C. for 3 h, after which it became homogenous. Cooling, concentration, and silica gel chromatography purification (0-50% gradient, ethyl acetate in hexanes) yielded (4-benzyloxy-2-methyl-phenoxy)-tert-butyldimethyl-silane 73 as a white solid: ¹H-NMR (400 MHz, CDCl₃) δ=7.42 (m, 2H), 7.37 (m, 2H), 7.31 (m, 1H), 6.79 (d, J=2.2 Hz, 1H), 6.67 (m, 2H), 4.99 (s, 2H), 2.18 (s, 3H), 1.01 (s, 9H), 0.18 (s, 6H). MS calcd. for C₂₀H₂₉O₂Si (M+H⁺) 329.2, found 329.2.

Step D: (4-Benzyloxy-2-methyl-phenoxy)-tert-butyl-dimethyl-silane 73 (5.03 g, 15.3 mmol) is dissolved in THF (30 mL). A 1 M solution of tetra-(n-butyl)ammonium fluoride in THF (18 mL, 18 mmol) is added. Then the mixture is stirred at rt for 4 h. Concentration to dryness and purification by silica gel chromatography (10-30% gradient, ethyl acetate in hexanes) yielded 4-benzyloxy-2-methyl-phenol 74: ¹H-NMR (400 MHz, CDCl₃) δ=7.42 (m, 4H), 7.31 (m, 1H), 6.78 (s, 1H), 6.69 (s, 2H), 4.99 (s, 2H), 2.27 (s, 3H).

Step E: 4-Benzyloxy-2-methyl-phenol 74 (3.06 g, 14.3 mmol) is dissolved in acetonitrile (60 mL). Powdered cesium carbonate (8.71 g, 26.7 mmol) is added to the vigorously stirred solution. 2-Bromo-2-methyl-propionic acid methyl ester (2.20 mL, 17.0 mmol) is added and the mixture is stirred at 60° C. for 6 h. Filtration and concentration yielded 2-(4-benzyloxy-2-methyl-phenoxy)-2-methyl-propionic acid methyl ester 75 (5.11 g, quant.) as an oil: ¹H-NMR (400 MHz, CDCl₃) δ=7.37 (m, 5H), 6.80 (d, J=2.4 Hz, 1H), 6.65 (d, J=2.8 Hz, 1H), 6.64 (s, 1H), 4.98 (5, 2H), 3.80 (s, 3H), 2.21 (s, 3H), 1.54 (s, 6H). MS calcd. for C₁₉H₂₂NaO₄ (M+Na⁺) 337.2, found 337.2.

Step F: 2-(4-Benzyloxy-2-methyl-phenoxy)-2-methyl-propionic acid methyl ester 75 (5.11 g, 14.3 mmol) is dissolved in ethanol (120 mL). The solution is degassed with nitrogen, then treated with a catalytic amount of 5% palladium black on carbon (1.50 g, 4 mol %). The solution is shaken under 60 psi hydrogen for 15 h. Filtration and concentration yielded an oil. Silica gel chromatography (hexanes to 60% ethyl acetate in hexanes) afforded 2-(4-hydroxy-2-methyl-phenoxy)-2-methyl-propionic acid methyl ester 76 (3.42 g, quant.) as an oil: ¹H-NMR (400 MHz, CDCl₃) δ=6.64 (d, J=3.0 Hz, 1H), 6.59 (d, J=8.7 Hz, 1H), 6.51 (dd, J=8.7, 3.1 Hz, 1H), 4.62 (s, 1H), 3.80 (s, 3H), 2.19 (s, 3H), 1.53 (s, 6H). MS calcd. for C₁₂H₁₆NaO₄ (M+Na⁺) 247.1, found 247.1.

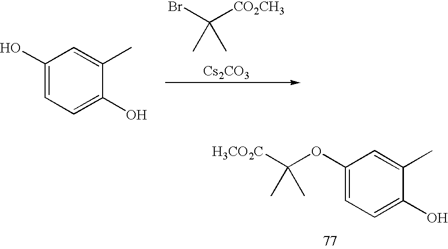

### Intermediate 77

### 2-(4-Hydroxy-2-methyl-phenoxy)-2-methyl-propionic Acid Methyl Ester

2-Methyl-hydroquinone (1.01 g, 8.13 mmol) is dissolved in acetonitrile (15 mL). Powdered cesium carbonate (2.86 g, 8.78 mmol) is added to the vigorously stirred solution. 2-Bromo-2-methyl-propionic acid methyl ester (1.10 mL, 8.50 mmol) dissolved in acetonitrile (5 mL) is added dropwise. The mixture is stirred at rt for 6 h. Filtration and concentration, followed by purification by silica gel chromatography (10-70% gradient, ethyl acetate in hexanes) yielded 2-(4-hydroxy-2-methyl-phenoxy)-2-methyl-propionic acid methyl ester 77 as and oil: ¹H-NMR (400 MHz, CDCl₃) δ=6.61 (m, 3H), 4.53 (s, 1H), 3.73 (s, 3H), 2.19 (s, 3H), 1.53 (s, 6H). The structure is confirmed by a NOESY experiment (the resonance at 1.53 ppm has a medium-strength positive nOe with the aromatic signals around 6.61 ppm, no nOe is observed

Step B: 2,5-Dimethylhydroquinone 79 (3.73 g, 27 mmol) is dissolved in dimethylformamide (20 mL) and acetonitrile (60 mL). Powdered cesium carbonate (9.16 g, 28.1 g) is added to the vigorously stirred solution, followed by 2-bromo-2-methyl-propionic acid methyl ester (3.50 mL, 27.0 mmol). The mixture is stirred at 75° C. for 18 h. Filtration and concentration, followed by purification by silica gel chromatography (5-30% gradient, ethyl acetate in hexanes) yielded 2-(4-hydroxy-2,5-dimethyl-phenoxy)-2-methyl-propionic acid methyl ester 80 as and oil. The chromatography also yielded recovered hydroquinone 79. 80: ¹H-NMR (400 MHz, CDCl₃) δ=6.57 (s, 1H), 6.50 (s, 1H), 4.44 (s, 1H), 2.15 (s, 3H), 2.14 (s, 3H), 1.52 (s, 6H). MS calcd. for C₁₃H₁₈NaO₄ (M+Na⁺) 261.1, found 261.1.

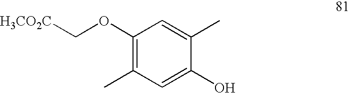

### Intermediate 81

### (4-Hydroxy-2,5-dimethyl-phenoxy)-acetic Acid Methyl Ester

Following the procedure for Intermediate 80, except substituting the appropriate bromoacetate, the title compound is prepared as a clear glass: ¹H-NMR (400 MHz, CDCl₃) δ=6.60 (s, 1H), 6.53 (s, 1H), 4.58 (s, 2H), 3.80 (s, 3H), 2.22 (s, 3H), 2.19 (s, 3H).

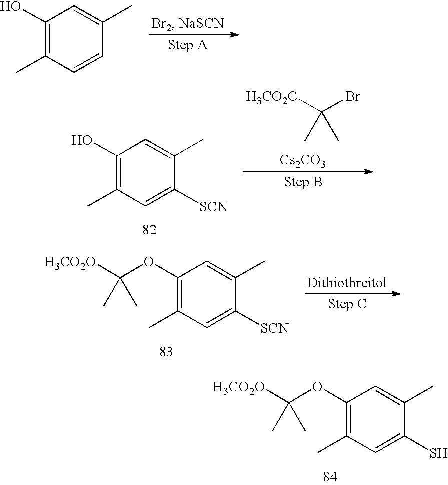

Intermediate 84: 2-(4-Mercapto-2,5-dimethyl-phenoxy)-2-methyl-propionic acid methyl ester.

Step A: 2,5-Dimethylphenol (10.04 g, 82.2 mmol) is dissolved in methanol (40 mL). Sodium thiocyanate (15.87 g, 195.8 mmol) and sodium bromide (7.37 g, 71.6 mmol) are added and the mixture is stirred at 0° C. Bromine (4.50 mL, 87.6 mmol) dissolved in methanol (40 mL) is added dropwise while stirring vigorously. Upon the completion of the addition, the mixture is stirred at 50° C. for 1 h. The mixture is cooled and concentrated. The residue is taken up in ethyl acetate and filtered. The filtrate is washed with saturated aqueous NaHCO₃, water, and brine, dried over Na₂SO₄ and concentrated to afford 2,5-dimethyl-4-thiocyanato-phenol 82 as an oil that solidified upon drying under high vacuum: ¹H-NMR (400 MHz, CDCl₃) δ=7.38 (s, 1H), 6.73 (s, 1H), 5.22 (s, 1H), 2.45 (s, 3H), 2.21 (s, 3H).

Step B: 2,5-Dimethyl-4-thiocyanato-phenol 82 (5.75 g, 32.1 mmol) is dissolved in acetonitrile (25 mL). Powdered cesium carbonate (15.32 g, 47.0 mmol) is added. Then 2-bromo-2-methyl-propionic acid methyl ester (4.50 mL, 34.8 mmol) is added and the mixture is stirred at 60° C. for 18 h. Filtration and concentration, followed by silica gel chromatography (0-50% ethyl acetate in hexanes) yielded 2-(2,5-dimethyl-4-thiocyanato-phenoxy)-2-methyl-propionic acid methyl ester 83 as an oil: ¹H-NMR (400 MHz, CDCl₃) (rotamers are present; the data given is for the most abundant isomer) δ=7.39 (s, 1H), 6.50 (s, 1H), 3.78 (s, 3H), 2.42 (s, 3H), 2.20 (s, 3H), 1.62 (s, 6H). MS calcd. for C₁₄H₁₇NNaO₃S (M+Na⁺) 302.1, found 302.1.

Step C: 2-(2,5-dimethyl-4-thiocyanato-phenoxy)-2-methyl-propionic acid methyl ester 83 (3.88 g, 13.9 mmol) is dissolved in methanol (50 mL). Potassium dihydrogenphosphate (0.23 g, 1.69 mmol), water (6 mL), and dithiothreitol (2.80 g, 18.2 mmol) are added and the mixture is stirred at reflux for 3 h. After cooling and concentration, the residue is taken up in ethyl acetate, washed with water and brine, dried over Na₂SO₄ and concentrated to yield an oil. Silica gel chromatography purification (0-65% ethyl acetate in hexanes) afforded 2-(4-mercapto-2,5-dimethyl-phenoxy)-2-methyl-propionic acid methyl ester 84 as a colourless oil: ¹H-NMR (400 MHz, CDCl₃) δ=7.09 (s, 1H), 6.47 (s, 1H), 3.79 (s, 1H), 3.10 (s, 1H), 2.24 (s, 3H), 2.15 (s, 3H), 1.56 (s, 6H).

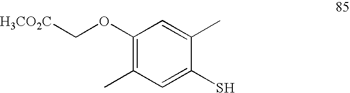

### Intermediate 85

### (4-Mercapto-2,5-dimethyl-phenoxy)-acetic Acid Methyl Ester

Following the procedure for Intermediate 84, except substituting the appropriate bromoacetate, the title compound is prepared as a clear liquid: ¹H-NMR (400 MHz, CDCl₃) δ=7.11 (s, 1H), 6.53 (s, 1H), 4.61 (s, 2H), 3.80 (s, 3H), 3.11 (s, 1H), 2.30 (s, 3H), 2.21 (s, 3H).

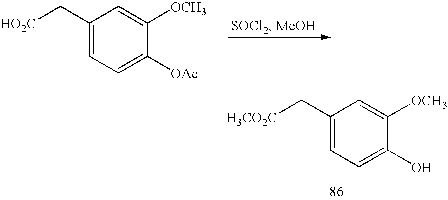

### Intermediate 86

### (4-Hydroxy-3-methoxy-phenyl)-acetic Acid Methyl Ester

(4-Acetoxy-3-methoxy-phenyl)-acetic acid (2.0 g, 8.9 mmol) is dissolved in methanol (15 mL). Thionyl chloride (1.5 mL, 20.6 mmol) is added dropwise with vigorous stirring. The mixture is stirred at 50° C. overnight. Cooling and concentration yielded (4-hydroxy-3-methoxy-phenyl)-acetic acid methyl ester 86 (1.8 g, quant.) as an oil: ¹H-NMR (400 MHz, CDCl₃) δ=6.86 (d, J=8.0 Hz, 1H), 6.80 (d, J=1.7 Hz, 1H), 6.76 (dd, J=8.0, 1.8 Hz, 1H), 5.10 (br. s, 1H), 3.88 (s, 3H), 3.69 (s, 3H), 3.55 (s, 2H).

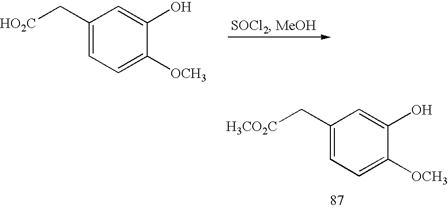

### Intermediate 87

### (3-Hydroxy-4-methoxy-phenyl)-acetic Acid Methyl Ester

(3-Hydroxy-4-methoxy-phenyl)-acetic acid (5.0 g, 27.4 mmol) is dissolved in methanol (30 mL). Thionyl chloride (2.0 mL, 27.5 mmol) is added dropwise with vigorous stirring. The mixture is stirred at 50° C. overnight. Cooling and concentration yielded (3-hydroxy-4-methoxy-phenyl)-acetic acid methyl ester 87 as an oil: ¹H-NMR (400 MHz, CDCl₃) δ=6.86 (d, J=2.0 Hz, 1H), 6.80 (d, J=8.2 Hz, 1H), 6.75 (dd, J=8.2, 2.0 Hz, 1H), 3.87 (s, 3H), 3.68 (s, 3H), 3.53 (s, 2H).

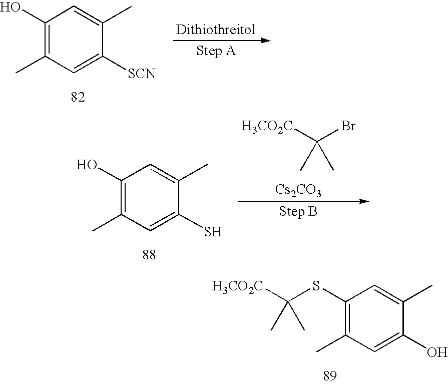

### Intermediate 89

### 2-(4-Hydroxy-2,5-dimethyl-phenylsulfanyl)-2-methyl-propionic Acid Methyl Ester

Step A: 2,5-Dimethyl-4-thiocyanato-phenol 82 (1.50 g, 8.4 mmol) is dissolved in methanol (30 mL). Potassium dihydrogenphosphate (0.32 g, 2.35 mmol), water (4 mL), and dithiothreitol (2.17 g, 14.1 mmol) are added and the mixture is stirred at reflux for 3 h. After cooling and concentration, the residue is taken up in ethyl acetate, washed with water and brine, dried over Na₂SO₄ and concentrated to yield an oil. Silica gel chromatography purification (0-65% ethyl acetate in hexanes) afforded 4-mercapto-2,5-dimethyl-phenol 88 as a colourless wax: ¹H-NMR (400 MHz, CDCl₃) δ=7.10 (s, 1H), 6.63 (s, 1H), 4.81 (s, 1H), 3.08 (s, 1H), 2.28 (s, 3H), 2.17 (s, 3H). MS calcd. for C₈H₁₁O₂S (M+H⁺) 155.1, found 155.0.

Step B: 4-Mercapto-2,5-dimethyl-phenol 88 (0.44 g, 2.85 mmol) is dissolved in acetonitrile (5 mL). Powdered cesium carbonate (1.55 g, 4.8 mmol) is added. Then 2-bromo-2-methyl-propionic acid methyl ester (0.350 mL, 2.7 mmol) is added and the mixture is stirred at 25° C. for 3 h. Filtration and concentration, followed by silica gel chromatography (10-50% ethyl acetate in hexanes) yielded 2-(4-hydroxy-2,5-dimethyl-phenylsulfanyl)-2-methyl-propionic acid methyl ester 89 as a wax: ¹H-NMR (400 MHz, CDCl₃) δ=7.17 (s, 1H), 6.66 (s, 1H), 4.94 (s, 1H), 3.67 (s, 3H), 2.36 (s, 3H), 2.18 (s, 3H), 1.46 (s, 6H). MS calcd. for C₁₃H₁₉O₃S (M+Na⁺) 255.1, found 255.1.

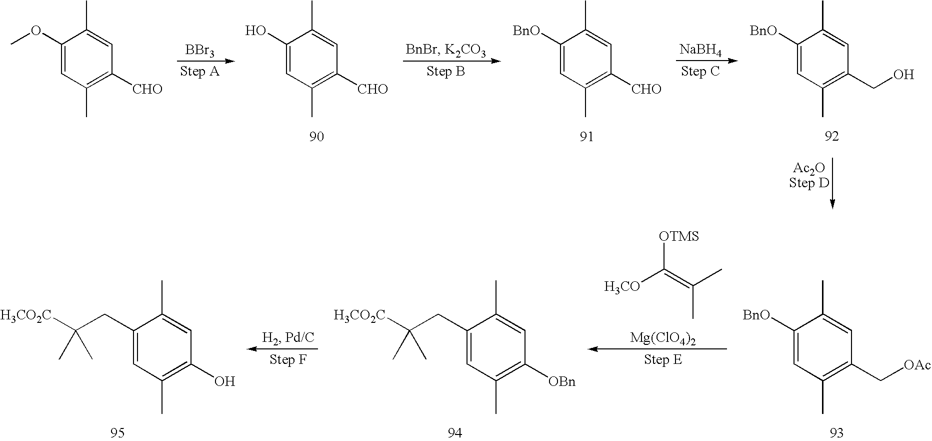

### Intermediate 95

### 3-(4-Hydroxy-2,5-dimethyl-phenyl)-2,2-dimethyl-propionic Acid Methyl Ester

Step A: 4-Methoxy-2,5-dimethyl-benzaldehyde (1.24 g, 7.55 mmol) is dissolved in dry dichloromethane (12 mL). Neat boron tribromide (1.75 g, 18.5 mmol) is added dropwise, with stirring. A tan-coloured precipitate started to form. The suspension is stirred at room temperature for 5 d. The homogenous mixture is poured over 150 g ice. After the ice melted, the solid phenol 90 is isolated by filtration and dried (1.28 g, quantitative). ¹H-NMR (400 MHz, dmso-d₆) δ=10.40 (s 1H), 9.98 (s, 1H), 7.54 (s, 1H), 6.68 (s, 1H), 3.36 (s, 1H), 2.49 (s, 3H), 2.13 (s, 3H).

Step B: 4-Hydroxy-2,5-dimethyl-benzaldehyde 90 (30.56 g, 0.2 mol) is dissolved in acetonitrile (150 mL). Benzyl bromide (24 mL, 0.2 mol) is added, followed by powdered potassium carbonate (36.92 g, 0.27 mol). The mixture is stirred at 60° C. for 18 h. Cooling and concentration, followed by silica gel chromatography (0-20% ethyl acetate in hexanes) yielded 4-benzyloxy-2,5-dimethyl-benzaldehyde 91 as a colorless oil. ¹H-NMR (400 MHz, CDCl₃) δ=10.13 (s, 1H), 7.61 (s, 1H), 7.43 (m, 5H), 6.72 (s, 1H), 5.15 (s, 2H), 2.63 (s, 3H), 2.28 (s, 3H). MS calcd. for C₁₆H₁₇O₂ (M+H⁺) 241.1, found 241.1.

Step C: 4-Benzyloxy-2,5-dimethyl-benzaldehyde 91 (4.77 g, 20 mmol) is dissolved in diethyl ether (30 mL). Sodium borohydride (1.0 g, 27 mmol) is added in one portion, followed by 5 mL absolute ethanol. The mixture is vigorously stirred for 3 h at room temperature, then carefully poured over 100 mL 1N aqueous HCl. Extraction with ethyl acetate, washing with water and brine, then concentration yielded (4-benzyloxy-2,5-dimethyl-phenyl)-methanol 92 as a soft solid. ¹H-NMR (400 MHz, CDCl₃) δ=7.39 (m, 5H), 7.11 (s, 1H), 6.73 (s, 1H), 5.07 (s, 2H), 4.61 (s, 2H), 2.35 (s, 3H), 2.25 (s, 3H).

Step D: (4-Benzyloxy-2,5-dimethyl-phenyl)-methanol 92 (4.79 g, 19.7 mmol) and ethyl diisopropylamine (6.0 mL, 34.4 mmol) are dissolved in dichloromethane (80 mL). Acetic anhydride (2.5 mL, 26.4 mmol) is added in one portion and the mixture is stirred at room temperature for 18 h. Washing with 1N HCl, water, saturated aqueous NaHCO₃, saturated aqueous NH₄Cl and brine, followed by drying over MgSO₄ and concentration yields acetic acid 4-benzyloxy-2,5-dimethyl-benzyl ester 93 as an oil (4.93 g, quant.). ¹H-NMR (400 MHz, CDCl₃) δ=7.39 (m, 5H), 7.11 (s, 1H), 6.73 (s, 1H), 5.07 (s, 2H), 5.04 (s, 2H), 2.32 (s, 3H), 2.24 (s, 3H), 2.07 (s, 3H).

Step E: Acetic acid 4-benzyloxy-2,5-dimethyl-benzyl ester 93 (0.56 g, 2 mmol) is dissolved in dry dichloromethane (5 mL). (1-Methoxy-2-methyl-propenyloxy)-trimethylsilane (1 mL, 5 mmol) and magnesium perchlorate (0.09 g, 0.4 mmol) are added and the suspension is stirred overnight. Filtration and silica gel chromatography (0-30% ethyl acetate in hexanes) yielded 3-(4-benzyloxy-2,5-dimethyl-phenyl)-2,2-dimethyl-propionic acid methyl ester 94 as an oil. ¹H-NMR (400 MHz, CDCl₃) δ=7.37 (m, 5H), 6.81 (s, 1H), 6.67 (s, 1H), 5.02 (s, 2H), 2.82 (s, 3H), 2.25 (s, 3H), 2.20 (s, 3H), 1.18 (s, 6H).

Step F: 3-(4-Benzyloxy-2,5-dimethyl-phenyl)-2,2-dimethyl-propionic acid methyl ester 94 (0.45 g, 1.4 mmol) is dissolved in ethanol (20 mL). Palladium black on carbon (5%; 0.16 g, 5 mol %) is added and the mixture is vigorously stirred under 1 atm. hydrogen for 18 h. Filtration and concentration yielded 3-(4-hydroxy-2,5-dimethyl-phenyl)-2,2-dimethyl-propionic acid methyl ester 95 as an oil (0.11 g, 34%). ¹H-NMR (400 MHz, CDCl₃) δ=6.75 (s, 1H), 6.56 (s, 1H), 3.67 (s, 3H), 2.80 (s, 2H), 2.20 (s, 3H), 2.16 (s, 3H), 1.17 (s, 6H).

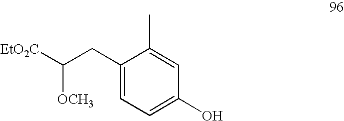

### Intermediate 96

### (±)-2-Methoxy-3-(4-hydroxy-2-methyl-phenyl)-propionic Acid Ethyl Ester

Following the procedure for Intermediate 46, except substituting the appropriate methoxyacetate, the title compound is prepared as a clear oil: ¹H-NMR (400 MHz, CDCl₃) δ=7.04 (d, J=8.4 Hz, 2H), 6.73 (d, J=8.4 Hz, 2H), 5.35 (s, 1H), 3.96 (dd, J=5.4, 7.3 Hz, 1H), 3.72 (s, 3H), 3.35 (s, 3H), 2.96 (m, 2H). MS calcd. for C₁₁H₁₄NaO₄ (M+Na⁺) 233.1, found 233.1.

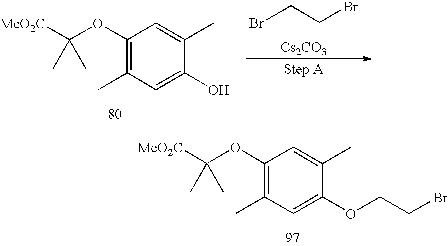

### Intermediate 97

### 2-[4-(2-Bromo-ethoxy)-2,5-dimethyl-phenoxy]-2-methyl-propionic Acid Methyl Ester

Step C: Intermediate 80 (0.25 g, 1.05 mmol), 1,2-dibromoethane (0.90 mL, 10.5 mmol) and Cs₂CO₃ (1.7 g, 5.25 mmol) are suspended in dry acetonitrile (7 mL). The mixture is heated to 80° C. overnight. The reaction mixture is cooled to room temperature, filtered and the solvent is removed in vacuo. The remainder is purified by flash chromatography (silica, DCM/MeOH gradient) to afford 2-[4-(2-bromo-ethoxy)-2-methyl-phenoxy]-2-methyl-propionic acid methyl ester 97 (0.24 g, 66%) as a colourless oil: ¹H-NMR (400 MHz, CDCl₃) δ=6.59 (s, 1H), 6.52 (s, 1H), 4.22 (t, J=6.2 Hz, 2H), 3.80 (s, 3H), 3-62 (t, J=6.2 Hz, 2H), 2.18 (s, 3H), 2.15 (s, 3H), 1.53 (s, 6H). MS calculated for C₁₅H₂₂BrO₄ (M+H⁺) 345.1, found 345.0.

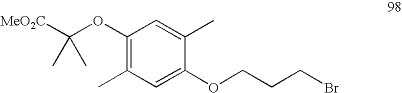

### Intermediate 98

### 2-[4-(3-Bromo-propoxy)-2,5-dimethyl-phenoxy]-2-methyl-propionic Acid Methyl Ester

Following the procedure for Intermediate 97, except substituting 1,3-dibromopropane for 1,2-dibromoethane, the title compound is prepared as a clear oil: ¹H-NMR (400 MHz, CDCl₃) δ=6.49 (s, 1H), 6.40 (s, 1H), 3.90 (t, J=5.7 Hz, 2H), 3.68 (s, 3H), 3.49 (t, J=6.5 Hz, 2H), 2.18 (m, 2H), 2.07 (s, 3H), 1.99 (s, 3H), 1.40 (s, 6H). MS calculated for C₁₆H₂₄BrO₄ (M+H⁺) 359.1, found 359.0.

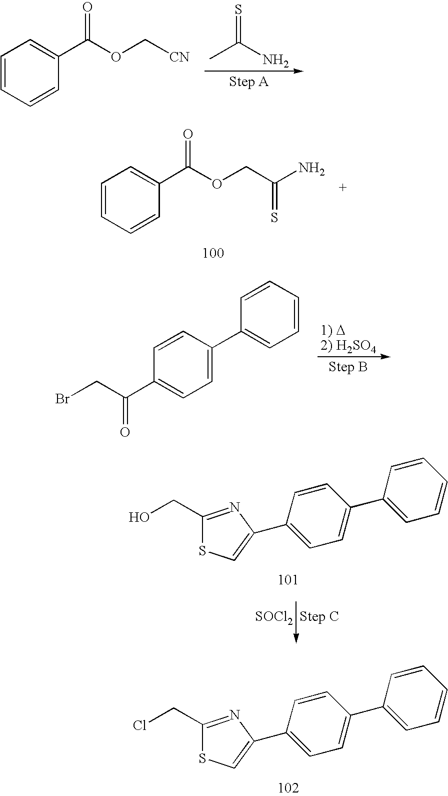

### Intermediate 102

### 4-Biphenyl-4-yl-2-chloromethyl-thiazole

Step A: Benzoyloxyacetonitrile (10 g, 62 mmol) and thioacetamide (11.6 g, 155 mmol are dissolved in DMF (40 mL). HCl (60 mL of 4N in dioxane, 235 mmol) is added and the mixture is heated at 100° C. for 2 h. The mixture is cooled and poured into water (100 mL) and extracted with EtOAc (80 mL). The organic layer is washed with water (100 mL), sat. NaHCO₃ (2×100 mL) then brine (100 mL), dried (MgSO₄), filtered and evaporated to give crude benzoic acid thiocarbamoylmethyl ester 100 which is used in Step B without further purification. MS calcd. for C₉H₁₀NO₂S (M+H⁺) 196.0, found 196.0.

Step B: Benzoic acid thiocarbamoylmethyl ester 100 (2.8 g, 14.3 mmol) and 1-biphenyl-4-yl-2-bromo-ethanone (3.9 g, 14.3 mmol) are dissolved in MeOH (20 mL) and heated at reflux for 16 h. Concentrated H₂SO₄ (2 mL) is added and the reflux continued for 24 h. The mixture is cooled and poured into water (100 mL) and extracted with EtOAc (2×80 mL). The organic layers are combined and washed with water (100 mL), sat. NaHCO₃ (2×100 mL) and brine (100 mL), dried (MgSO₄), filtered and evaporated and purified by flash chromatography (EtOAc/Hexanes gradient) to afford (4-Biphenyl-4-yl-thiazol-2-yl)-methanol 101 (0.86 g, 23%). MS calcd. for C₁₆H₁₄NOS (M+H⁺) 268.1, found 268.1.

Step C: (4-Biphenyl-4-yl-thiazol-2-yl)-methanol 101 (860 mg, 3.22 mmol) is dissolved in dry THF (20 mL), then thionyl chloride (0.47 mL, 6.43 mmol) is added and the mixture is stirred at rt for 2 h. The mixture is poured into a saturated solution of NaHCO₃ (20 mL) and extracted with EtOAc (20 mL). The organic layer is washed with brine, dried (MgSO₄), filtered and evaporated to give 4-Biphenyl-4-yl-2-chloromethyl-thiazole 102 (0.50 g, 55%) as a beige powder. ¹H-NMR (400 MHz, CDCl₃) 7.96 (d, J=8.4 Hz, 2H), 7.66 (m, 4H), 7.55 (s, 1H), 7.46 (t, J=8.0 Hz, 2H), 7.37 (t, J=8.8 Hz, 1H), 4.94 (s, 2H). MS calcd. for C₁₆H₁₃ClNS (M+H⁺) 286.0, found 286.0.

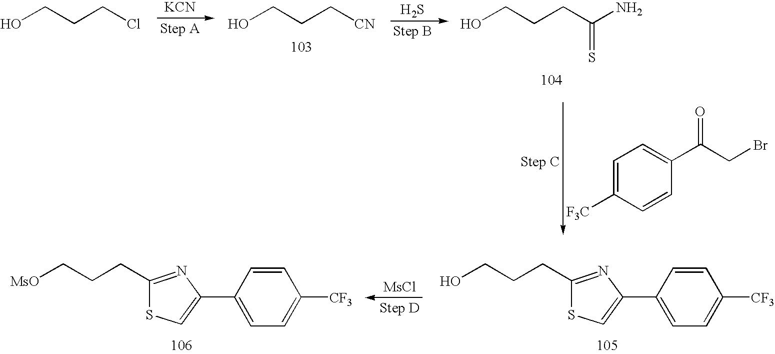

### Intermediate 106

### Methanesulfonic acid 3-[4-(4-trifluoromethyl-phenyl)-thiazol-2-yl]-propyl Ester

Step A: Potassium cyanide (25.4 g, 0.39 mol) is suspended into a mixture of ethanol (120 mL) and water (40 mL). 3-Chloropropanol (26.8 mL, 0.32 mol) is added and the mixture is heated to reflux for 90 min. After filtering insoluble material the remaining oil is distilled (100 mTorr, 75° C.) to give 4-hydroxy-butyronitrile 103 (10 g, 37%) as a colourless oil: ¹H-NMR (400 MHz, CDCl₃) 3.69 (t, J=5.8 Hz, 2H), 2.46 (t, J=7.1 Hz, 2H), 1.84 (quint, J=6.5 Hz, 2H).

Step B: 4-hydroxy-butyronitrile 103 (7.5 g, 90 mmol) is dissolved in pyridine (60 mL). Triethylamine (25 mL) is added and the solution is flushed with nitrogen. Then hydrogen sulfide gas is bubbled steadily through the solution for 3.5 h. Concentration yielded 4-hydroxy-thiobutyramide 104 as a yellow oil: ¹H-NMR (400 MHz, CDCl₃) 3.78 (t, J=5.8 Hz, 2H), 2.51 (t, J=7.1 Hz, 2H), 1.91 (quint, J=65 Hz, 2H).

Step C: 4-hydroxy-thiobutyramide 104 (7.0 g, 59 mmol) and 2-bromo-1-(4-trifluoromethyl-phenyl)-ethanone (15.7 g, 59 mmol) are dissolved in EtOH (50 mL) and heated to 80° C. for 5 h. The mixture is concentrated and purified by flash chromatography (EtOAc/Hexanes gradient) to afford 3-[4-(4-trifluoromethylphenyl)-thiazol-2-yl]-propan-1-ol 105 as a colorless solid: MS calcd. for C₁₃H₁₃F₃NOS (M+H⁺) 288.1, found 288.1.

Step D: 3-[4-(4-Trifluoromethyl-phenyl)-thiazol-2-yl]-propan-1-ol 105 (2.5 g, 8.7 mmol) and ethyldiisopropylamine (3.5 mL, 21.2 mmol) are dissolved in dichloromethane (35 mL). Mesyl chloride (0.85 mL, 11.0 mmol) are added dropwise and the mixture is stirred at rt overnight. Then the mixture is diluted with DCM and washed with H₂O and brine. The organic layer is dried (MgSO₄), filtered and concentrated to afford the mesylate 106 as a light-brown oil: MS calcd. for C₁₄H₁₅F₃NO₃S₂ (M+H⁺) 366.0, found 366.1.

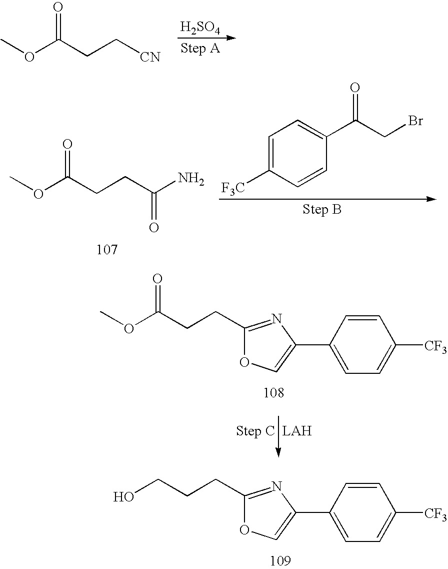

### Intermediate 109

### 3-[4-(4-Trifluoromethyl-phenyl)-oxazol-2-yl]-propan-1-ol

Step A: 3-Cyano-propionic acid methyl ester (3.0 g, 2.7 mmol) is dissolved in methanol (6 mL) and stirred in an ice-bath. Concentrated sulfuric acid (12 mL) is added slowly, then the ice-bath is removed and the mixture is stirred overnight at rt. The acid is quenched by adding solid NaHCO3 and water, the product is extracted with EtOAc and dried to give succinamic acid methyl ester 107 as a white solid: ¹H-NMR (400 MHz, CDCl₃) 5.64 (s, br, NH2), 3.51 (s, 3H), 2.49 (t, J=6.7 Hz, 2H), 2.39 (t, J=6.7 Hz, 2H).

Step B: Succinamic acid methyl ester 107 (0.7 g, 5.3 mmol) and 2-bromo-1-(4-trifluoromethyl-phenyl)-ethanone (0.71 g, 2.7 mmol) are stirred neat at 80° C. for 4 h. Then the mixture is dissolved in EtOAc and washed with H₂O and brine. The organic layer is dried (MgSO₄), filtered and concentrated. The mixture is purified by flash chromatography (EtOAc/Hexanes gradient) to afford 3-[4-(4-trifluoromethyl-phenyl)-oxazol-2-yl]-propionic acid methyl ester 108 as a colorless oil: ¹H-NMR (400 MHz, CDCl₃) 7.84 (s, 1H), 7.76 (d, J=8.2 Hz, 2H), 7.58 (d, J=8.2 Hz, 2H), 3.67 (s, 3H), 3.10 (t, J=7.5 Hz, 2H), 2.83 (t, J=7.5 Hz, 2H). MS calcd. for C₁₄H₁₃F₃NO₃ (M+H⁺) 300.1, found 300.0.

Step C: The 3-[4-(4-trifluoromethyl-phenyl)-oxazol-2-yl]propionic acid methyl ester 108 (0.26 g, 0.87 mmol) is placed in a flame-dried flask, dissolved in THF/DCM 1:1 (5 mL) and flushed with argon. After cooling to 0° C., lithium aluminiumtetrahydride (0.87 mL 1M in THF, 0.87 mmol) is added dropwise over a period of 45 min. Sodium sulfate decahydrate (0.28 g, 0.87 mmol) is added slowly in portions, the ice-bath is removed and the mixture is stirred at rt for 20 min. Then the mixture is filtered through celite and washed with DCM and EtOAc. The solvents are removed in vacuo to yield 3-[4-(4-trifluoromethyl-phenyl)-oxazol-2-yl]-propan-1-ol 109 (0.24 g, quant.) as a colorless glass: ¹H-NMR (400 MHz, CDCl₃) 7.84 (s, 1H), 7.74 (d, J=8.2 Hz, 2H), 7.57 (d, J=8.2 Hz, 2H), 3.73 (t, J=5.6 Hz, 2H), 2.91 (t, J=7.1 Hz, 2H), 2.02 (m, 2H). MS calcd. for C₁₃H₁₃F₃NO₂ (M+H⁺) 272.1, found 272.0.

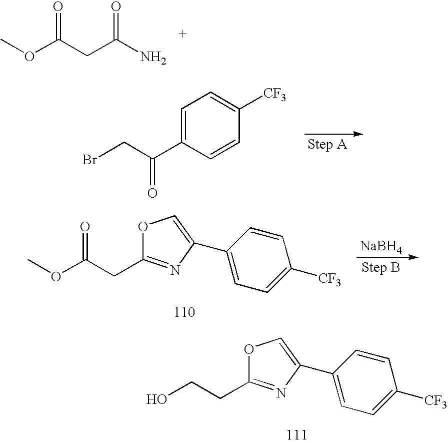

### Intermediate 111

### 2-[4-(4-Trifluoromethyl-phenyl)-oxazol-2-yl]-ethanol

Step A: Malonamic acid methyl ester (22.0 g, 187 mmol) and 2-Bromo-1-(4-trifluoromethyl-phenyl)-ethanone (10 g, 37.4 mmol) are heated to 130° C. with stirring for 2 h. Water (30 mL) is added and the product is extracted with EtOAc (2×40 mL). The organic layers are combined, washed with water (30 mL) and brine (30 mL), dried (MgSO₄), filtered, concentrated and purified by flash chromatography (EtOAc/Hexanes gradient) to afford [4-(4-Trifluoromethyl-phenyl)-oxazol-2-yl]-acetic acid methyl ester 110 as a colorless oil: ¹H-NMR (400 MHz, CDCl₃) 7.98 (s, 1H), 7.83 (d, J=8.0 Hz, 2H), 7.65 (d, J=8.0 Hz, 2H), 3.94 (s, 2H), 3.78 (s, 3H). MS calcd. for C₁₃H₁₁F₃NO₃ (M+H⁺) 286.1, found 286.1.

Step B: [4-(4-Trifluoromethyl-phenyl)-oxazol-2-yl]-acetic acid methyl ester 110 (1.8 g, 6.31 mmol) is dissolved in dry THF (40 mL). Sodium borohydride (377 mg, 10.1 mmol) is added and the mixture is heated at reflux for 16 h. The reaction is poured into 1 N HCl (20 mL) and extracted with EtOAc (2×50 mL). The organic layers are combined and washed with 1 N HCl, dried (MgSO₄), filtered and evaporated to afford the title compound 111: ¹H-NMR (400 MHz, CDCl₃) 7.94 (s, 1H), 7.82 (d, J=8.0 Hz, 2H), 7.65 (d, J=8.0 Hz, 2H), 4.10 (m, 3H), 3.77 (s, 1H). MS calcd. for C₁₂H₁₁F₃NO₂ (M+H⁺) 258.1, found 258.1.

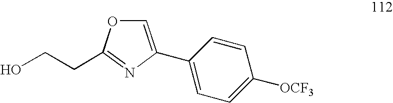

### Intermediate 112

### 2-[4-(4-Trifluoromethoxy-phenyl)-oxazol-2-yl]-ethanol

Following the procedure of Intermediate 111, except substituting 2-Bromo-1-(4-trifluoromethoxy-phenyl)-ethanone for 2-Bromo-1-(4-trifluoromethyl-phenyl)-ethanone, the title compound is prepared a red oil. ¹H-NMR (400 MHz, CDCl₃) 7.78 (s, 1H), 7.66 (d, J=8.0 Hz, 2H), 7.17 (d, J=8.0 Hz, 2H), 4.01 (t, J=5.6 Hz, 2H), 2.98 (t, J=5.6 Hz, 2H). MS calcd. for C₁₂H₁₁F₃NO₃ (M+H⁺) 274.1, found 274.1.

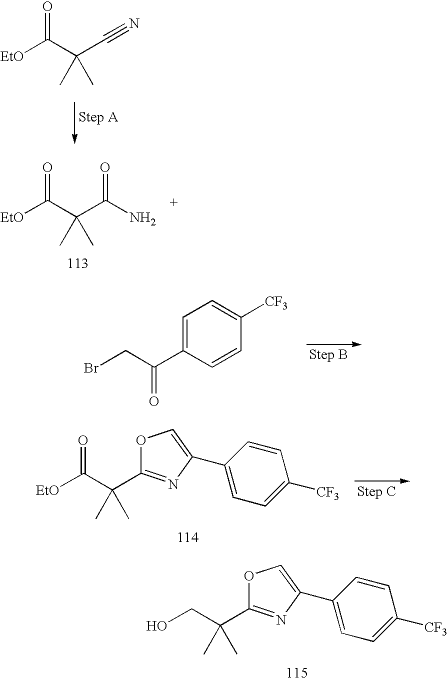

### Intermediate 115

### 2-Methyl-2-[4-(4-trifluoromethyl-phenyl)-oxazol-2-yl]-propan-1-ol

Step A: Cyano-dimethyl-acetic acid ethyl ester (3 g, 21.2 mmol) is dissolved in EtOH (5 mL), then concentrated H₂SO₄ (20 mL) is added and the mixture is stirred at rt for 16 h. The reaction is poured into water (100 mL) and NaHCO₃ is added portionwise to neutral pH. Then the mixture is extracted with EtOAc (3×60 mL). The organic layers are combined and washed with brine, dried (MgSO₄), filtered and evaporated to afford 2,2-dimethyl-malonamic acid ethyl ester 113 as a clear oil. ¹H-NMR (400 MHz, CDCl₃) 4.11 (q, J=7.2 Hz, 2H), 1.38 (s, 6H), 1.20 (t, J=7.2 Hz, 3H).

Step B: 2,2-Dimethyl-malonamic acid ethyl ester 113 (3.0 g, 11.4 mmol) and 2-bromo-1-(4-trifluoromethyl-phenyl)-ethanone (2.7 g, 17.1 mmol) are heated to 130° C. with vigorous stirring for 2 h. Then water (30 mL) is added and the product is extracted with EtOAc (2×40 mL). The organic layers are combined, washed with water (30 mL) and brine (30 mL), dried (MgSO₄), filtered, concentrated and purified by flash chromatography (EtOAc/Hexanes gradient) to afford 2-methyl-2-[4-(4-trifluoromethyl-phenyl)-oxazol-2-yl]-propionic acid ethyl ester 114 as a colorless oil. MS calcd. for C₁₆H₁₇F₃NO₃ (M+H⁺) 328.1, found 328.1.

Step C: 2-Methyl-2-[4-(4-trifluoromethyl-phenyl)-oxazol-2-yl]-propionic acid ethyl ester 114 (2.6 g, 7.94 mmol) is dissolved in dry THF (40 mL). Sodium borohydride (1.5 g, 40.2 mmol) is added and the mixture is heated at reflux for 64 h. The reaction is poured into 1 N HCl (20 mL) and extracted with EtOAc (2×50 mL). The organic layers are combined and washed with 1 N HCl, dried (MgSO₄), filtered and evaporated to afford the title compound 115: ¹H-NMR (400 MHz, CDCl₃) 7.85 (s, 1H), 7.74 (d, J=8.0 Hz, 2H), 7.57 (d, J=8.0 Hz, 2H), 3.69 (s, 2H), 1.32 (s, 6H). MS calcd. for C₁₄H₁₅F₃NO₂ (M+H⁺) 286.1, found 286.1.

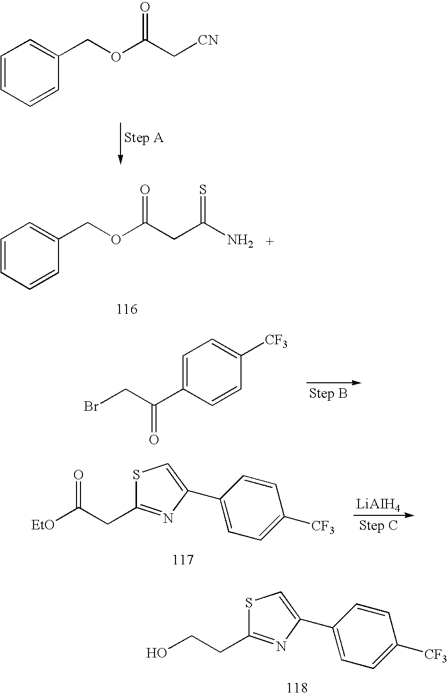

### Intermediate 118

### 2-[4-(4-Trifluoromethyl-phenyl)-thiazol-2-yl]-ethanol

Step A: Cyano-acetic acid benzyl ester (2 g, 11.4 mmol) and thioacetamide (2.14 g, 28.5 mmol are dissolved in DMF (7 mL). HCl (10.8 mL of 4N in dioxane, 43.3 mmol) is added and the mixture is heated at 100° C. for 2 h. The mixture is cooled, poured into water (30 mL) and extracted with EtOAc (50 mL). The organic layer is washed with water (20 mL), sat. NaHCO₃ (2×20 mL) then brine (20 mL), dried (MgSO₄), filtered and evaporated to give crude Thiocarbamoyl-acetic acid benzyl ester 116 which is used in Step B without further purification. MS calcd. for C₁₀H₁₂NO₂S (M+H⁺) 210.1, found 210.0.

Step B: Thiocarbamoyl-acetic acid benzyl ester 116 (0.8 g, 3.8 mmol) and 2-Bromo-1-(4-trifluoromethyl-phenyl)-ethanone (1.0 g, 3.81 mmol) are dissolved in EtOH (20 mL) and heated at reflux for 2 h. The mixture is cooled and filtered to afford [4-(4-Trifluoromethyl-phenyl)-thiazol-2-yl]-acetic acid ethyl ester 117. MS calcd. for C₁₄H₁₃F₃NO₂S (M+H⁺) 316.1, found 316.0.

Step C: [4-(4-Trifluoromethyl-phenyl)-thiazol-2-yl]-acetic acid ethyl ester 117 (397 mg, 1.26 mmol) is dissolved in dry THF (10 mL), then LiAlH₄ (1.26 mL of 1 M in THF, 1.26 mmol) is added and the mixture is stirred at rt for 1 h. The reaction is quenched by slow addition of water (5 mL) and extracted with EtOAc (20 mL). The organic layer is washed with 1 N HCl (10 mL), dried (MgSO₄), filtered, evaporated and purified by flash column chromatography (EtOAc/Hexane gradient) to afford 2-[4-(4-Trifluoromethyl-phenyl)-thiazol-2-yl]-ethanol 118 as a white powder. ¹H-NMR (400 MHz, CDCl₃) 7.97 (d, J=8.0 Hz, 2H), 7.66 (d, J=8.0 Hz, 2H), 7.48 (s, 1H), 5.47 (m, 1H), 4.09 (t, J=5.6 Hz, 2H), 3.28 (t, J=6.0 Hz, 2H). MS calcd. for C₁₂H₁₁F₃NOS (M+H⁺) 274.0, found 274.0.

### Intermediate 119

### 4-(4-(trifluoromethyl)phenyl)thiazole-2-thiol

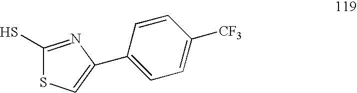

2-Bromo-1-(4-trifluoromethyl-phenyl)-ethanone (10 g, 37.4 mmol) and ammonium dithiocarbamate (4.2 g, 37.4 mmol) are dissolved in dry acetone (100 mL) and heated at reflux for 2 h. The solution is cooled and stirred at rt for 2 h, then filtered and washed with acetone to give 4-(4-(trifluoromethyl)phenyl)thiazole-2-thiol 119 as a white solid: 3H-NMR (400 MHz, DMSO-d6) δ=7.99 (d, J=8.4 Hz, 2H), 7.79 (d, J=8.4 Hz, 2H), 7.47 (s, 1H). MS calculated for C₁₅H₁₃N₂S (M+H⁺) 262.1, found 262.0.

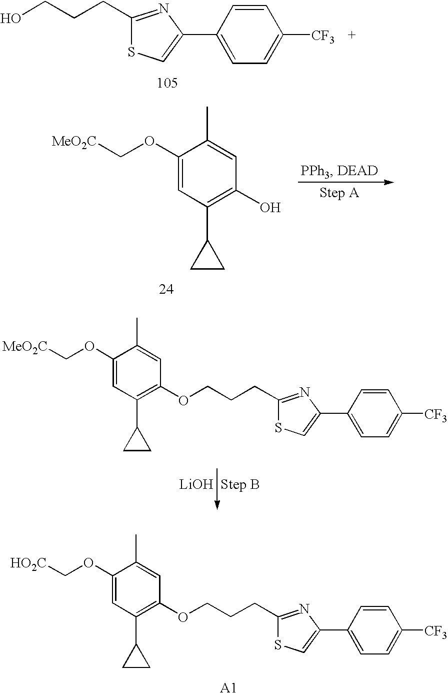

### Example A1

### (5-Cyclopropyl-2-methyl-4-{3-(4-(4-trifluoromethyl-phenyl)-thiazol-2-yl-propoxy}-phenoxy)-acetic Acid

Step A: Intermediate 105 (25 mg, 0.09 mmol), intermediate 24 (26 mg, 0.11 mmol) and triphenylphosphine (35 mg, 0.14 mmol) are dissolved in dry DCM (1 mL) and cooled to 0° C. After the slow addition of diethyl azodicarboxylate (29 μL, 0.18 mmol) the solution is stirred at room temperature overnight. The solvent is removed to afford crude (5-cyclopropyl-2-methyl-4-{3-[4-(4-trifluoromethyl-phenyl)-thiazol-2-yl]-propoxy}-phenoxy)-acetic acid methyl ester which is used without further purification in step B.

Step B: The crude (5-cyclopropyl-2-methyl-4-{3-[4-(4-trifluoromethyl-phenyl)-thiazol-2-yl]-propoxy}-phenoxy)-acetic acid methyl ester is dissolved in THF (1.8 mL), a solution of 1 M LiOH in H₂O (0.6 mL) is added and the mixture is stirred overnight at room temperature. The mixture is acidified with 1 M HCl (0.7 mL), DCM (10 mL) is added and the organic layer washed with H₂O (3×5 mL). The organic layer is dried (MgSO₄), filtered, concentrated and purified on reverse phase HPLC (H₂O/MeCN gradient) to afford the title compound A1 as a colorless glass: ¹H-NMR (400 MHz, CD₃OD) δ=7.77 (d, J=7.9 Hz, 2H), 7.44 (d, J=7.9 Hz, 2H), 7.24 (s, 1H), 6.45 (s, 1H), 6.09 (s, 1H), 4.36 (s, 2H), 3.86 (t, J=6.0 Hz, 2H), 3.11 (t, J=7.7 Hz, 2H), 2.16 (m, 2H), 2.01 (s, 3H), 1.91 (m, 1H), 0.69 (m, 2H), 0.37 (m, 2H). MS calcd. for C₂₅H₂₅F₃NO₄S (M+H⁺) 492.1, found 492.2.

By repeating the procedures described in the above examples, using appropriate starting materials, the following compounds of Formula I, as identified in Table 1, are obtained.

TABLE 1

| Compound | Compound | Physical Data |
|---|---|---|
| Number | Structure | ¹H NMR and/or MS (m/z) |
| A2 | 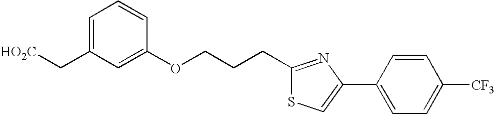 | ¹H-NMR (400 MHz, CDCl₃) δ = 7.75 (d, J = 7.9 Hz, 2H), 7.48 (d, J = 7.9 Hz, 2H), 7.06 (s, 1H), 7.05 (t, J = 7.5 Hz, 1H), 6.65 (m, 3H), 3.89 (t, J = 5.4 Hz, 2H), 3.43 (s, 2H), 3.15 (t, J = 7.1 Hz, 2H), 2.16 (m, 2H). MS calcd. for C₂₁H₁₉F₃NO₃S (M + H⁺) 422.1, found 422.1. |
| A3 | 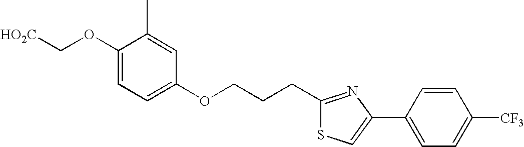 | MS calcd. for C₂₂H₂₁F₃NO₄s (M + H⁺) 452.1, found 452.1. |
| A4 | 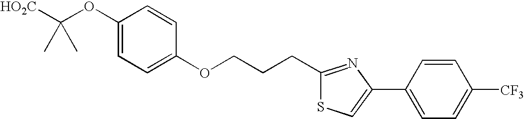 | ¹H-NMR (400 MHz, CDCl₃) δ = 7.90 (d, J = 7.7 Hz, 2H), 7.62 (d, J = 7.7 Hz, 2H), 7.42 (s, 1H), 6.85 (d, J = 7.0 Hz, 2H), 6.76 (d, J = 7.0 Hz, 2H), 4.00 (t, J = 4.6 Hz, 2H), 3.28 (t, J = 6.9 Hz, 2H), 2.29 (m, 2H), 1.48 (s, 6H). MS calcd. for C₂₃H₂₃F₃NO₄S (M + H⁺) 466.1, found 466.2. |
| A5 | 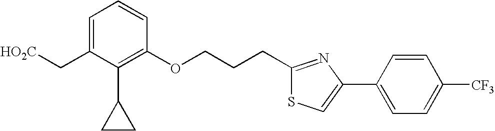 | ¹H-NMR (400 MHz, CDCl₃) δ = 8.01 (d, J = 8.0 Hz, 2H), 7.71 (d, J = 8.0 Hz, 2H), 7.50 (s, 1H), 7.18 (t, J = 8.0 Hz, 1H), 6.88 (d, J = 8.0 Hz, 1H), 6.83 (d, J = 8.0 Hz, 1H), 4.11 (t, J = 5.7 Hz, 2H), 3.99 (s ,2H), 3.43 (t, J = 7.4 Hz, 2H), 2.43 (m, 2H), 1.65 (m, 1H), 1.05 (m, 2H), 0.71 (m, 2H). MS calcd. for C₂₄H₂₃F₃NO₃s (M + H⁺) 462.1, found 462.2. |
| A6 | 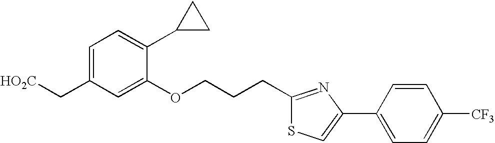 | ¹H-NMR (400 MHz, CDCl₃) δ = 7.86 (d, J = 8.1 Hz, 2H), 7.59 (d, J = 8.1 Hz, 2H), 7.37 (s, 1H), 6.69 (m, 2H), 4.04 (t, J = 5.9 Hz, 2H), 3.51 (s, 2H), 3.31 (t, J = 7.5 Hz, 2H), 2.32 (m, 2H), 2.03 (m, 1H), 0.82 (m, 2H), 0.56 (m, 2H). MS calcd. for C₂₄H₂₃F₃NO₃S (M + H⁺) 462.1, found 462.2. |
| A7 | 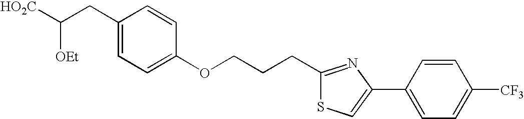 | ¹H-NMR (400 MHz, CDCl₃) δ = 7.97 (d, J = 7.9 Hz, 2H), 7.66 (d, J = 7.9 Hz, 2H), 7.45 (s, 1H), 7.14 (d, J = 7.2 Hz, 2H), 6.82 (d, J = 7.2 Hz, 2H), 4.04 (m, 3H), 3.58 (m, 1H), 3.46 (m, 3H), 3.28 (t, J = 7.0 Hz, 2H), 3.07 (m, 1H), 2.95 (m, 1H), 2.34 (m, 2H), 1.17 (t, J = 7.0 Hz, 3H). MS calcd. for C₂₄H₂₅F₃NO₄S (M + H⁺) 480.1, found 480.2. |
| A8 | 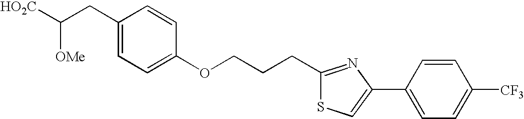 | MS calcd. for C₂₃H₂₃F₃NO₄S (M + H⁺) 466.1, found 466.1. |
| A9 | 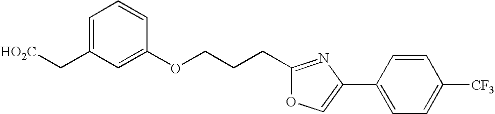 | ¹H-NMR (400 MHz, CDCl₃) δ = 7.90 (s, 1H), 7.81 (d, J = 8.1 Hz, 2H), 7.64 (d, J = 8.1 Hz, 2H), 7.23 (t, J = 7.9 Hz, 1H), 6.83 (m, 3H), 4.08 (t, J = 6.0 Hz, 2H), 3.60 (s, 2H), 3.05 (t, J = 7.4 Hz, 2H), 2.31 (m, 2H). MS calcd. for C₂₁H₁₉F₃NO₄ (M + H⁺) 406.1, found 406.1. |
| A10 | 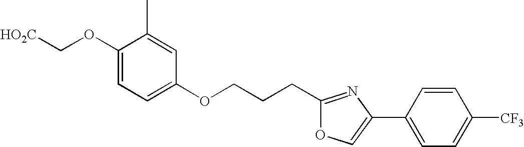 | ¹H-NMR (400 MHz, CDCl₃) δ = 7.95 (s, 1H), 7.81 (d, J = 7.3 Hz, 2H), 7.64 (d, J = 7.3 Hz, 2H), 7.25 (s, 1H), 6.83 (m, 3H), 6.67 (m, 3H), 4.60 (s, 2H), 4.01 (m, 2H), 3.05 (m, 2H), 2.28 (m, 2H), 2.25 (s, 3H). MS calcd. for C₂₂H₂₁F₃NO₅ (M + H⁺) 436.1, found 436.1. |
| A11 | 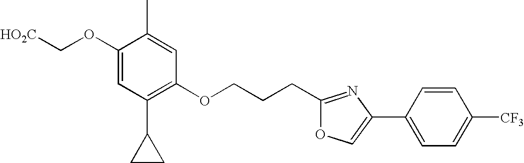 | ¹H-NMR (400 MHz, CDCl₃) δ = 7.91 (s, 1H), 7.82 (d, J = 8.2 Hz, 2H), 7.65 (d, J = 8.2 Hz, 2H), 6.67 (s, 1H), 6.31 (s, 1H), 4.57 (s, 2H), 4.07 (t, J = 5.9 Hz, 2H), 3.09 (t, J = 7.6 Hz, 2H), 2.34 (m, 2H), 2.23 (s, 3H), 2.11 (m, 1H), 0.90 (m, 2H), 0.58 (m, 2H). MS calcd. for C₂₅H₂₅F₃NO₅ (M + H⁺) 476.2, found 476.1. |
| A12 | 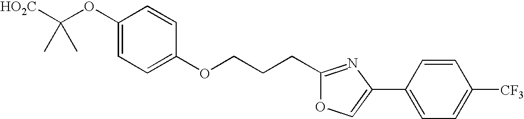 | ¹H-NMR (400 MHz, CDCl₃) δ = 7.91 (s, 1H), 7.82 (d, J = 8.3 Hz, 2H), 7.65 (d, J = 8.3 Hz, 2H), 6.90 (d, J = 9.0 Hz, 2H), 6.80 (d, J = 9.0 Hz, 2H), 4.05 (t, J = 6.0 Hz, 2H), 3.05 (t, J = 7.4 Hz, 2H), 2.31 (m, 2H), 1.53 (s, 6H). MS calcd. for C₂₃H₂₃F₃NO₅ (M + H⁺) 450.2, found 450.1. |
| A13 | 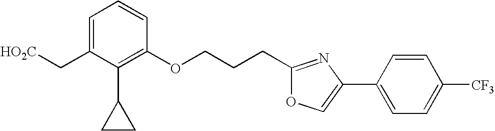 | ¹H-NMR (400 MHz, CDCl₃) δ = 7.91 (s, 1H), 7.82 (d, J = 8.0 Hz, 2H), 7.65 (d, J = 8.0 Hz, 2H), 7.14 (t, J = 8.0 Hz, 1H), 6.84 (d, J = 7.5 Hz, 1H), 6.79 (d, J = 8.0 Hz, 1H), 4.07 (t, J = 5.9 Hz, 2H), 3.94 (s, 2H), 3.12 (t, J = 7.4 Hz, 2H), 2.35 (m, 2H), 1.62 (m, 1H), 1.01 (m, 2H), 0.66 (m, 2H). MS calcd. for C₂₄H₂₃F₃NO₄ (M + H⁺) 446.2, found 446.1. |
| A14 | 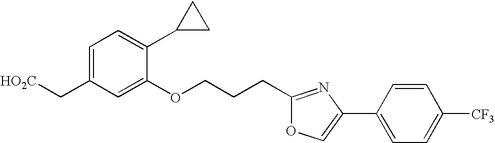 | MS calcd. for C₂₄H₂₃F₃NO₄ (M + H⁺) 446.2, found 446.1. |
| A15 | 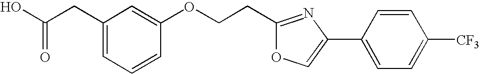 | ¹H-NMR (400 MHz, CDCl₃) δ = 7.99 (s, 1H), 7.88 (d, J = 8.0 Hz, 2H), 7.70 (d, J = 8.0 Hz, 2H), 6.93 (m, 4H), 4.47 (t, J = 6.8 Hz, 2H), 3.68 (s, 2H), 3.39 (t, J = 6.4 Hz, 2H). MS calcd. for C₂₀H₁₇F₃NO₄ (M + H⁺) 392.1, found 392.0. |
| A16 | 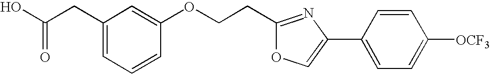 | ¹H-NMR (400 MHz, CDCl₃) δ = 7.78 (s, 1H), 7.67 (d, J = 8.8 Hz, 2H), 7.17 (d, J = 8.8 Hz, 2H), 6.80 (m, 4H), 4.32 (t, J = 6.8 Hz, 2H), 3.56 (s, 2H), 3.25 (t, J = 6.4 Hz, 2H). MS calcd. for C₂₀H₁₇F₃NO₅ (M + H⁺) 408.1, found 408.1. |
| A17 | 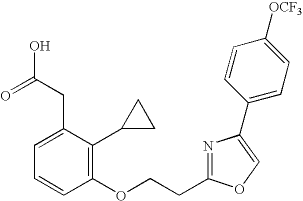 | ¹H-NMR (400 MHz, CDCl₃) δ = 7.78 (s, 1H), 7.66 (d, J = 8.8 Hz, 2H), 7.17 (d, J = 8.8 Hz, 2H), 7.08 (t, J = 7.6 Hz, 1H), 6.76 (t, J = 7.6 Hz, 2H), 4.32 (t, J = 6.4 Hz, 2H), 3.83 (s, 2H), 3.37 (t, J = 6.4 Hz, 2H), 1.45 (m, 1H), 0.79 (m, 2H), 0.43 (m, 2H). MS calcd. for C₂₃H₂₁F₃NO₅ (M + H⁺) 448.1, found 448.1. |
| A18 | 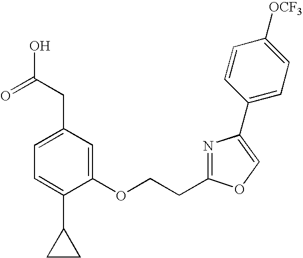 | ¹H-NMR (400 MHz, CDCl₃) δ = 7.78 (s, 1H), 7.67 (d, J = 8.8 Hz, 2H), 7.18 (d, J = 8.4 Hz, 2H), 7.08 (t, J = 7.6 Hz, 1H), 6.73 (m, 3H), 4.37 (t, J = 6.8 Hz, 2H), 3.53 (s, 2H), 3.29 (t, J = 6.8 Hz, 2H), 1.98 (m, 1H), 0.76 (m, 2H), 0.50 (m, 2H). MS calcd. for C₂₃H₂₁F₃NO₅ (M + H⁺) 448.1, found 448.1. |
| A19 | 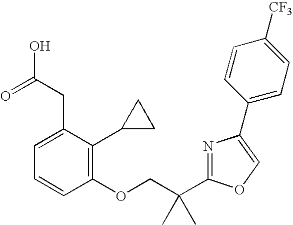 | ¹H-NMR (400 MHz, CDCl₃) δ = 7.90 (s, 1H), 7.84 (d, J = 8.0 Hz, 2H), 7.63 (d, J = 8.4 Hz, 2H), 7.14 (t, J = 7.6 Hz, 1H), 6.81 (t, J = 7.2 Hz, 2H), 4.13 (s, 2H), 3.89 (s, 2H), 1.60 (s, 6H), 1.49 (m, 1H), 0.84 (m, 2H), 0.44 (m, 2H). MS calcd. for C₂₅H₂₅F₃NO₄ (M + H⁺) 460.2, found 460.2. |
| A20 | 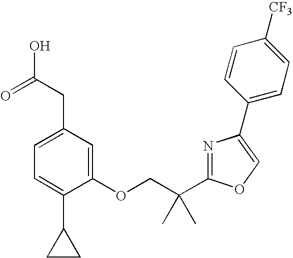 | ¹H-NMR (400 MHz, CDCl₃) δ = 7.78 (s, 1H), 7.72 (d, J = 8.0 Hz, 2H), 7.52 (d, J = 8.4 Hz, 2H), 6.67 (m, 3H), 4.05 (s, 2H), 3.49 (s, 2H), 1.87 (m, 1H), 1.49 (s, 6H), 0.66 (m, 2H), 0.41 (m, 2H). MS calcd. for C₂₅H₂₅F₃NO₄ (M + H⁺) 460.2, found 460.1. |
| A21 | 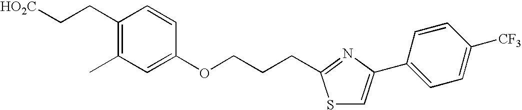 | ¹H-NMR (400 MHz, CDCl₃) δ = 7.99 (d, J = 8.0 Hz, 2H), 7.66 (d, J = 8.0 Hz, 2H), 7.46 (s, 1H), 7.06 (d, J = 8.0 Hz, 1H), 6.73 (s, 1H), 6.70 (d, J = 8.0 Hz, 1H), 4.06 (t, J = 5.8 Hz, 2H), 3.27 (t, J = 7.4 Hz, 2H), 2.90 (t, J = 7.9 Hz, 2H), 2.61 (t, J = 7.7 Hz, 2H), 2.34 (m, 2H), 2.29 (s, 3H). MS calcd. for C₂₃H₂₃F₃NO₃S (M + H⁺) 450.2, found 450.1. |
| A22 | 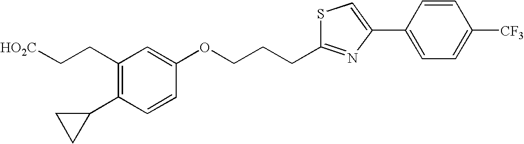 | ¹H-NMR (400 MHz, CDCl₃) δ = 7.98 (d, J = 6.9 Hz, 2H), 7.66 (d, J = 6.9 Hz, 2H), 7.45 (s, 1H), 6.94 (d, J = 8.6 Hz, 1H), 6.78 (d, J = 1.0 Hz, 1H), 6.69 (dd, J = 8.6, 1.0 Hz, 1H), 4.10 (t, J = 5.8 Hz, 2H), 3.25 (t, J = 7.2 Hz, 2H), 3.15 (t, J = 7.2 Hz, 2H), 2.73 (br s, 2H), 2.30 (m, 2H), 1.84 (m, 1H), 0.90 (m, 2H), 0.60 (m, 2H). MS calcd. for C₂₅H₂₅F₃NO₃S (M + H⁺) 477.2, found 477.2. |
| A23 | 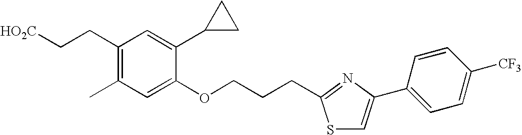 | ¹H-NMR (400 MHz, CDCl₃) δ = 8.00 (d, J = 8.0 Hz, 2H), 7.66 (d, J = 8.0 Hz, 2H), 7.46 (s, 1H), 6.63 (s, 1H), 6.62(s, 1H), 4.10 (t, J = 5.9 Hz, 2H), 3.32 (t, J = 7.5 Hz, 2H), 2.86 (t, J = 7.8 Hz, 2H), 2.58 (t, J = 7.8 Hz, 2H), 2.39 (m, 2H), 2.26 (s, 3H), 2.11 (m, 1H), 0.89 (m, 2H), 0.62 (m, 2H). MS calcd. for C₂₆H₂₇F₃NO₃S (M + H⁺) 490.2, found 490.2. |
| A24 | 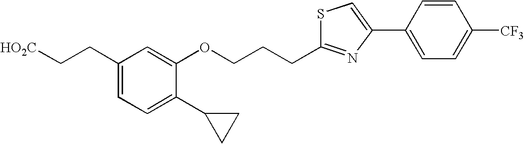 | ¹H-NMR (400 MHz, CDCl₃) δ = 7.97 (d, J = 8.0 Hz, 2H), 7.66 (d, J = 7.8 Hz, 2H), 7.44 (s, 1H), 6.77 (d, J = 8.4 Hz, 2H), 6.73 (s, 1H), 6.72 (d, J = 8.4 Hz, 2H), 4.18 (t, J = 6.0 Hz, 2H), 3.30 (t, J = 7.7 Hz, 2H), 2.92 (t, J = 7.7 Hz, 2H), 2.64 (t, J = 7.4 Hz, 2H), 2.37 (m, 2H), 2.13 (m, 1H), 0.89 (m, 2H), 0.63 (m, 2H). MS calcd. for C₂₅H₂₅F₃NO₃S (M + H⁺) 476.2, found 476.1. |
| A25 | 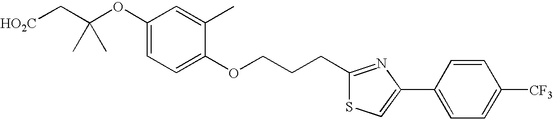 | ¹H-NMR (400 MHz, CDCl₃) (rotomers are present): δ = 7.98 (d, J = 8.2 Hz, 2H), 7.67 (d, J = 8.2 Hz, 2H), 7.46 (s, 1H), 6.81 (m, 1H), 6.70 (m, 2H), 4.05 (m, 2H), 3.30 (m, 2H), 2.37 (m, 2H), 2.22 (d, 3H), 1.55 (s, 3H), 1.53 (s, 3H). MS calcd. for C₂₄H₂₅F₃NO₄S (M + H⁺) 480.2, found 480.2. |
| A26 | 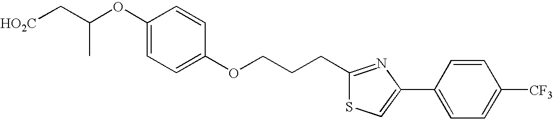 | ¹H-NMR (400 MHz, CDCl₃) δ = 7.99 (d, J = 8.0 Hz, 2H), 7.66 (d, J = 8.3 Hz, 2H), 7.46 (s, 1H), 7.10 (d, J = 8.6 Hz, 2H), 6.84 (d, J = 8.6 Hz, 2H), 4.07 (t, J = 6.0 Hz, 2H), 3.27 (t, J = 7.4 Hz, 2H), 3.00 (dd, J = 13.4, 6.7 Hz, 1H), 2.73 (m, 1H), 2.64 (dd, J = 13.4, 7.7 Hz, 1H), 2.34 (m, 2H), 1.18 (d, J = 6.9 Hz, 3H). MS calcd. for C₂₃H₂₃F₃NO₃S (M + H⁺) 450.2, found 450.1. |
| A27 | 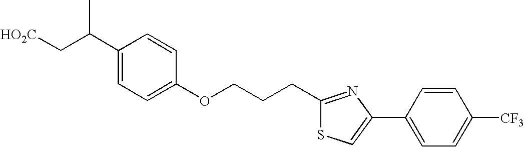 | ¹H-NMR (400 MHz, CDCl₃) δ = 7.99 (d, J = 8.0 Hz, 2H), 7.66 (d, J = 8.3 Hz, 2H), 7.45 (s, 1H), 7.14 (d, J = 7.4 Hz, 2H), 6.85 (d, J = 7.4 Hz, 2H), 4.07 (t, J = 6.0 Hz, 2H), 3.00 (dd, J = 15.4, 7.1 Hz, 1H), 2.56 (dd, J = 15.4, 8.0 Hz, 1H), 2.34 (m, 2H), 1.30 (d, J = 7.0 Hz, 3H). MS calcd. for C₂₃H₂₃F₃NO₃S (M + H⁺) 450.2, found 450.1. |
| A28 | 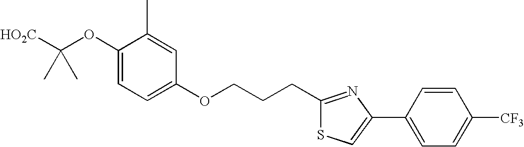 | ¹H-NMR (400 MHz, CDCl₃) δ = 7.94 (d, J = 8.1 Hz, 2H), 7.68 (d, J = 8.2 Hz, 2H), 7.47 (s, 1H), 6.81 (d, J = 8.8 Hz, 1H), 6.73 (d, J = 2.9 Hz, 1H); 6.63 (dd, J = 8.8, 3.0 Hz, 1H), 4.03 (t, J = 6.0 Hz, 2H), 3.32 (t, J = 7.5 Hz, 2H), 2.33 (m, 2H), 2.22 (s, 3H), 1.56 (s, 6H). MS calcd. for C₂₄H₂₅F₃NO₄S (M + H⁺) 480.2, found 480.1. |

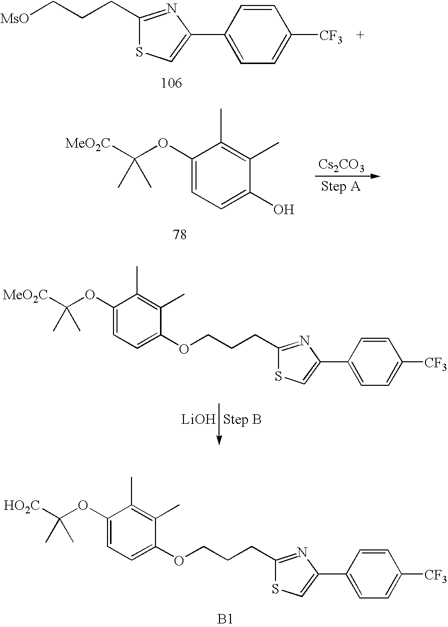

### Example B1

### 2-(2,3-Dimethyl-4-{3-[4-(4-trifluoromethylphenyl)-thiazol-2-yl]-propoxy}-phenoxy)-2-methyl-propionic Acid

Step A: Methanesulfonic acid 3-[4-(4-trifluoromethyl-phenyl)-thiazol-2-yl]-propyl ester 106 (37 mg, 0.10 mmol) and 2-(4-hydroxy-2,3-dimethyl-phenoxy)-2-methyl-propionic acid methyl ester 78 (23.8 mg, 0.10 mmol) are dissolved in dry acetonitrile (2 mL). Powdered cesium carbonate (50 mg, 0.16 mmol) is added and the solution is vigorously stirred at 55° C. for 6 h. Filtration and concentration yielded crude 2-(2,3-dimethyl-4-{3-[4-(4-trifluoromethyl-phenyl)-thiazol-2-yl]propoxy}-phenoxy)-2-methyl-propionic acid methyl ester which is used without further purification in Step B.

Step B: The crude 2-(2,3-dimethyl-4-{3-[4-(4-trifluoromethylphenyl)-thiazol-2-yl]-propoxy}-phenoxy)-2-methyl-propionic acid methyl ester is dissolved in dioxane (1.5 mL). Lithium hydroxide monohydrate (0.10 g, excess) and water (0.5 mL) are added. The mixture is stirred at 60° C. for 2 h. Cooling to room temperature, concentration, acidification with 1 N HCl, and purification on reversed-phase HPLC (H₂O/MeCN gradient) afforded the title compound B1 as a colorless oil: ¹H-NMR (400 MHz, CDCl₃) δ=7.99 (d, J=8.1 Hz, 2H), 7.67 (d, J=8.1 Hz, 2H), 7.46 (s, 1H), 6.72 (d, J=8.8 Hz, 1H), 6.61 (d, J=8.7 Hz, 1H), 4.05 (t, J=6.0 Hz, 2H), 3.30 (t, J=7.4 Hz, 2H), 2.37 (m, 2H), 2.18 (s, 6H), 1.54 (s, 6H). ¹⁹F-NMR (376 MHz, CDCl₃) δ=−62.49. MS calcd. for C₂₅H₂₇F₃NO₄S (M+H⁺) 494.2, found 494.2.

By repeating the procedures described in the above examples, using appropriate starting materials, the following compounds of Formula I, as identified in Table 2, are obtained.

TABLE 2

| Compound | Compound | Physical Data |
|---|---|---|
| Number | Structure | ¹H NMR and/or MS (m/z) |
| B2 | 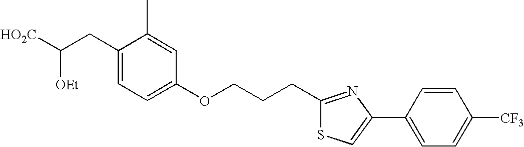 | ¹H-NMR (400 MHz, CDCl₃) δ = 7.99 (d, J = 8.1 Hz, 2H), 7.67 (d, J = 8.1 Hz, 2H), 7.46 (s, 1H), 7.11 (d, J = 8.3 Hz, 1H), 6.73 (d, J = 2.6 Hz, 1H), 6.69 (dd, J =8.3, 2.6 Hz, 1H), 4.07 (t, J = 6.0 Hz, 2H), 4.02 (dd, J = 8.5, 4.1 Hz, 1H), 3.54 (m, 1H), 3.36 (m, 1H), 3.28 (t, J = 7.5 Hz, 2H), 3.12 (dd, J = 14.3, 4.0 Hz, 1H), 2.94 (dd, J = 14.4, 8.4 Hz, 1H), 2.35 (m, 2H), 2.33 (s, 3H), 1.14 (t, J = 7.0 Hz, 3H). ¹⁹F- NMR (376 MHz, CDCl₃) δ = −62.50. MS calcd. for C₂₅H₂₇F₃NO₄S (M + H⁺) 494.2, found 494.2. |
| B3 | 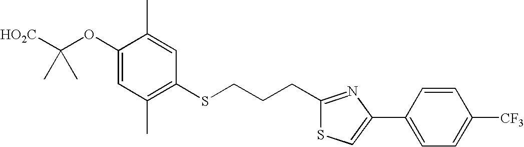 | ¹H-NMR (400 MHz, CDCl₃) δ = 7.97 (d, J = 8.1 Hz, 2H), 7.67 (d, J = 8.1 Hz, 2H), 7.45 (s, 1H), 7.16 (s, 1H), 6.68 (s, 1H), 3.20 (t, J = 6.0 Hz, 2H), 2.96 (t, J = 7.4 Hz, 2H), 2.34 (s, 3H), 2.17 (s, 3H), 2.12 (m, 2H), 1.60 (s, 6H). ¹⁹F-NMR (376 MHz, CDCl₃) δ = −62.51. MS calcd. for C₂₅H₂₇F₃NO₃S₂ (M + H⁺) 510.2, found 510.1. |
| B4 | 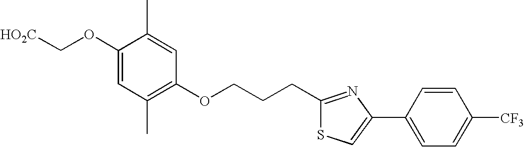 | ¹H-NMR (400 MHz, CDCl₃) δ = 7.96 (d, J = 8.1 Hz, 2H), 7.67 (d, J = 8.1 Hz, 2H), 7.46 (s, 1H), 6.65 (s, 1H), 6.61 (s, 1H), 4.61 (s, 2H), 4.04 (t, J = 6.0 Hz, 2H), 3.32 (t, J = 7.4 Hz, 2H), 2.35 (m, 2H), 2.24 (s, 3H), 2.19 (s, 3H). ¹⁹F-NMR (376 MHz, CDCl₃) δ = −62.54. MS calcd. for C₂₃H₂₃F₃NO₄S (M + H⁺) 466.2, found 466.1. |
| B5 | 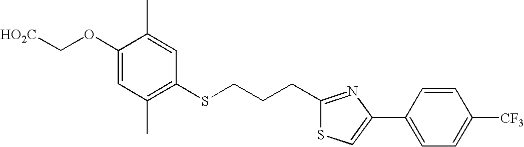 | ¹H-NMR (400 MHz, CDCl₃) δ = 7.94 (d, J = 8.1 Hz, 2H), 7.67 (d, J = 8.1 Hz, 2H), 7.45 (s, 1H), 7.20 (s, 1H), 6.59 (s, 1H), 4.67 (s, 2H), 3.23 (t, J = 6.0 Hz, 2H), 2.91 (t, J = 7.4 Hz, 2H), 2.40 (m, 2H), 2.22 (s, 3H), 2.12 (s, 3H). ¹⁹F-NMR (376 MHz, CDCl₃) δ = −62.54. MS calcd. for C₂₃H₂₃F₃NO₄S (M + H⁺) 466.2, found 466.1. |
| B6 | 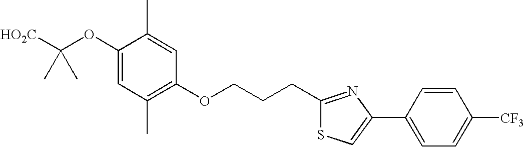 | ¹H-NMR (400 MHz, CDCl₃) δ = 7.98 (d, J = 8.1 Hz, 2H), 7.67 (d, J = 8.1 Hz, 2H), 7.46 (s, 1H), 6.71 (s, 1H), 6.62 (s, 1H), 4.05 (t, J = 6.0 Hz, 2H), 3.30 (t, J = 7.4 Hz, 2H), 2.37 (s, 3H), 2.20 (s, 3H), 2.17 (m, 2H), 1.54 (s, 6H). ¹⁹F-NMR (376 MHz, CDCl₃) δ = −62.51. MS calcd. for C₂₅H₂₇F₃NO₄S (M + H⁺) 494.2, found 494.1. |
| B7 | 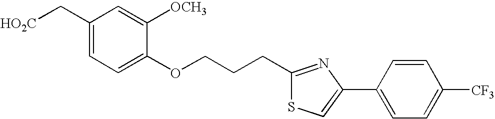 | ¹H-NMR (400 MHz, CDCl₃) δ = 7.96 (d, J = 8.1 Hz, 2H), 7.68 (d, J = 8.1 Hz, 2H), 7.48 (s, 1H), 6.84 (m, 3H), 4.39 (t, J = 6.3 Hz, 2H), 3.87 (s, 3H), 3.63 (s, 3H), 3.25 (t, J = 7.5 Hz, 2H), 2.34 (m, 2H). ¹⁹F-NMR (376 MHz, CDCl₃) δ = −62.56. MS calcd. for C₂₂H₂₁F₃NO₄S (M + H⁺) 452.2, found 452.2. |
| B8 | 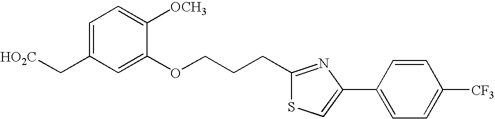 | ¹H-NMR (400 MHz, CDCl₃) δ = 7.97 (d, J = 8.1 Hz, 2H), 7.67 (d, J = 8.1 Hz, 2H), 7.46 (s, 1H), 6.85 (m, 3H), 4.16 (t, J = 6.3 Hz, 2H), 3.86 (s, 3H), 3.59 (s, 3H), 3.31 (t, J = 7.5 Hz, 2H), 2.40 (m, 2H). ¹⁹F-NMR (376 MHz, CDCl₃) δ = −62.51. MS calcd. for C₂₂H₂₁F₃NO₄S (M + H⁺) 452.2, found 452.2. |
| B9 | 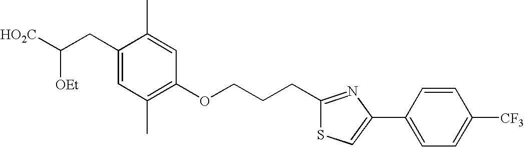 | MS calcd. for C₂₆H₂₉F₃NO₄S (M + H⁺) 508.2, found 508.1. |
| B10 | 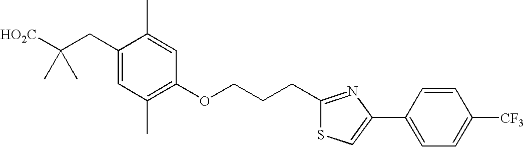 | ¹H-NMR (400 MHz, CDCl₃) δ = 7.92 (d, J = 8.2 Hz, 2H), 7.69 (d, J = 8.2 Hz, 2H), 7.47 (s, 1H), 6.88 (s, 1H), 6.60 (s, 1H), 4.06 (t, J = 6.0 Hz, 2H), 3.38 (t, J = 7.4 Hz, 2H), 2.87 (s, 2H), 2.36 (m, 2H), 2.26 (s, 3H), 2.14 (s, 3H), 1.21 (s, 6H). ¹⁹F-NMR (376 MHz, CDCl₃) δ = −62.63. MS calcd. for C₂₆H₂₈F₃NO₃S (M + H⁺) 492.2, found 492.1. |
| B11 | 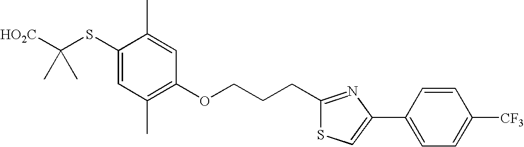 | ¹H-NMR (400 MHz, CDCl₃) δ = 7.93 (d, J = 8.2 Hz, 2H), 7.68 (d, J = 8.2 Hz, 2H), 7.47 (s, 1H), 7.24 (s, 1H), 6.69 (s, 1H), 4.06 (t, J = 6.0 Hz, 2H), 3.35 (t, J = 7.4 Hz, 2H), 2.44 (s, 3H), 2.38 (m, 2H), 2.14 (s, 3H), 1.47 (s, 6H). ¹⁹F-NMR (376 MHz, CDCl₃) δ = −62.59. MS calcd. for C₂₅H₂₇F₃NO₃S₂ (M + H⁺) 510.2, found 510.2. |

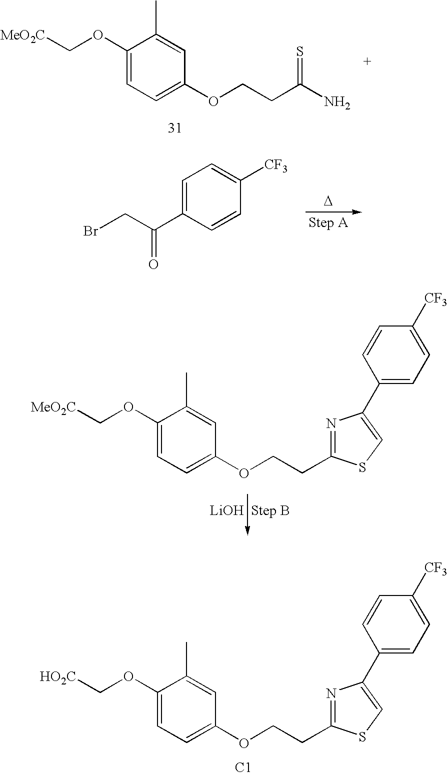

### Example C1

### (2-Methyl-4-{2-[4-(4-trifluoromethyl-phenyl)-thiazol-2-yl]-ethoxy}-phenoxy)-acetic Acid

Step A: [2-Methyl-4-(2-thiocarbamoyl-ethoxy)-phenoxy]-acetic acid methyl ester 31 (22 mg, 0.08 mmol) and 2-bromo-1-(4-trifluoromethyl-phenyl)-ethanone (20 mg, 0.075 mmol) are dissolved in MeOH (1 mL) and heated at 160° C. by subjection to microwave irradiation in a sealed tube for 10 minutes to afford crude (2-methyl-4-{2-[4-(4-trifluoromethyl-phenyl)thiazol-2-yl]-ethoxy}-phenoxy)-acetic acid methyl ester which is used without further purification in step B.

Step B: To the crude (2-methyl-4-(2-[4-(4-trifluoromethylphenyl)-thiazol-2-yl]-ethoxy)-phenoxy)-acetic acid methyl ester in MeOH is added THF (2 mL) and 1 N LiOH (1 mL). The mixture is stirred at rt for 16 h, then acidified with 1 N HCl and extracted with EtOAc (10 mL). The organic layer is dried (MgSO₄), filtered, concentrated and purified on reverse phase HPLC (H₂O/MeCN gradient) to afford the title compound Cl as a colorless glass: ¹H-NMR (400 MHz, CDCl₃) δ=8.21 (d, J=7.6 Hz, 2H), 7.88 (d, J=7.6 Hz, 2H), 7.71 (s, 1H), 7.03 (s, 1H), 6.93 (s, 2H), 4.84 (s, 2H), 4.55 (t, J=6.4 Hz, 2H), 3.74 (t, J=6.4 Hz, 2H), 2.39 (s, 3H). MS calcd. for C₂₁H₁₈F₃NO₄S (M+H⁺) 438.1, found 438.1.

By repeating the procedures described in the above examples, using appropriate starting materials, the following compounds of Formula I, as identified in Table 3, are obtained.

TABLE 3

| | | Physical Data |
|---|---|---|
| Compound | Compound | ¹H NMR 400 MHz (DMSO-d₆) |
| Number | Structure | and/or MS (m/z) |
| C2 | 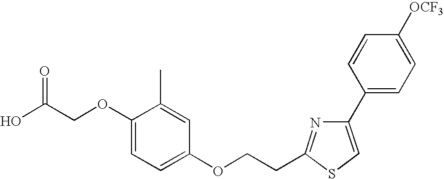 | ¹H-NMR (400 MHz, CDCl₃) δ = 7.78 (d, J = 8.8 Hz, 2 H), 7.27 (s, 1 H), 7.14 (d, J = 8.8 Hz, 2 H), 6.69 (s, 1 H), 6.59 (s, 2 H), 4.51 (s, 2 H), 4.21 (t, J = 6.4 Hz, 2 H), 3.44 (t, J = 6.4 Hz, 2 H), 2.16 (s, 3 H). MS calcd. for C₂₁H₁₉F₃NO₅S (M + H⁺) 454.1, found 454.1. |
| C3 | 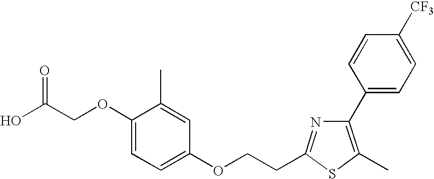 | ¹H-NMR (400 MHz, CDCl₃) δ = 7.66 (s, 4 H), 6.75 (s, 1 H), 6.65 (s, 2 H), 4.57 (s, 2 H), 4.22 (t, J = 6.0 Hz, 2 H), 3.48 (t, J = 6.0 Hz, 2 H), 2.49 (s, 3 H), 2.22 (s, 3 H). MS calcd. for C₂₂H₂₁F₃NO₄S (M + H⁺) 452.1, found 452.1. |
| C4 | 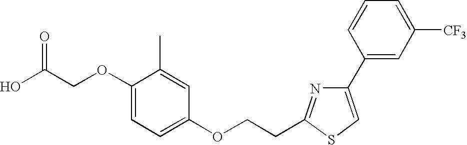 | ¹H-NMR (400 MHz, CDCl₃) δ = 7.97 (s, 1 H), 7.90 (d, J = 7.2 Hz, 1 H), 7.47 (d, J = 7.6 Hz, 1 H), 7.42 (t, J = 7.2 Hz, 1 H), 7.35 (s, 1 H), 6.67 (s, 1 H), 6.57 (s, 2 H), 4.49 (s, 2 H), 4.20 (t, J = 6.0 Hz, 2 H), 3.45 (t, J = 6.0 Hz, 2 H), 2.14 (s, 3 H). MS calcd. for C₂₁H₁₉F₃NO₄S (M + H⁺) 438.1, found 452.1. |
| C5 | 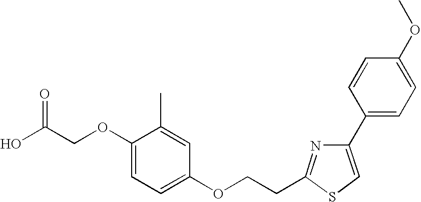 | ¹H-NMR (400 MHz, CDCl₃) δ = 8.03 (d, J = 8.8 Hz, 1 H), 7.89 (d, J = 8.4 Hz, 2 H), 7.02 (m, 3 H), 6.87 (s, 1 H), 6.77 (s, 2 H), 4.69 (s, 2 H), 4.39 (t, J = 6.0 Hz, 2 H), 3.92 (s, 3 H), 3.67 (t, J = 6.0 Hz, 2 H), 2.34 (s, 3 H). MS calcd. for C₂₁H₂₂NO₅S (M + H⁺) 400.1, found 400.2. |
| C6 | 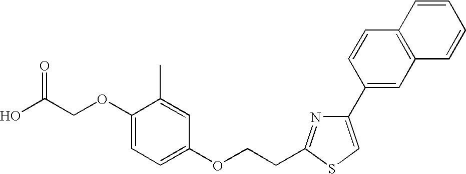 | ¹H-NMR (400 MHz, CDCl₃) δ = 8.44 (s, 1 H), 7.88 (m, 4 H), 7.48 (m, 3 H), 6.80 (s, 1 H), 6.70 (s, 2 H), 4.61 (s, 2 H), 4.34 (t, J = 6.0 Hz, 2 H), 3.62 (t, J = 6.0 Hz, 2 H), 2.26 (s, 3 H). MS calcd. for C₂₄H₂₂NO₄S (M + H⁺) 420.1, found 420.2. |
| C7 | 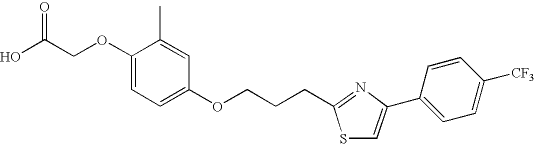 | ¹H-NMR (400 MHz, CDCl₃) δ = 7.87 (d, J = 7.6 Hz, 2 H), 7.54 (d, J = 7.6 Hz, 2 H), 7.34 (s, 1 H), 6.65 (s, 1 H), 6.57 (m, 2 H), 4.50 (s, 2 H), 3.92 (t, J = 6.0 Hz, 2 H), 3.16 (t, J = 6.0 Hz, 2 H), 2.21 (t, J = 6.4 Hz, 2 H), 2.15 (s, 3 H). MS calcd. for C₂₂H₂₁F₃NO₄S (M + H⁺) 452.1, found 452.1. |
| C8 | 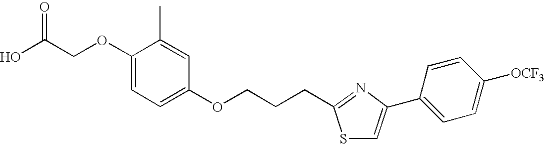 | ¹H-NMR (400 MHz, CDCl₃) δ = 8.10 (d, J = 8.4 Hz, 2 H), 7.58 (s, 1 H), 7.49 (d, J = 8.4 Hz, 2 H), 6.98 (s, 1 H), 6.92 (m, 2 H), 4.85 (s, 2 H), 4.26 (t, J = 6.0 Hz, 2 H), 3.54 (t, J = 6.0 Hz, 2 H), 2.55 (t, J = 6.8 Hz, 2 H), 2.49 (s, 3 H). MS calcd. for C₂₂H₂₁F₃NO₅S (M + H⁺) 468.1, found 468.1. |
| C9 | 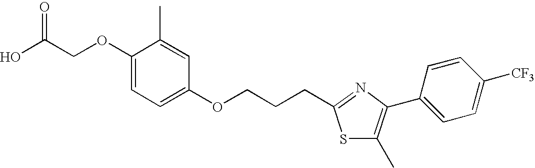 | ¹H-NMR (400 MHz, CDCl₃) δ = 7.55 (s, 4 H), 6.58 (s, 1 H), 6.50 (m, 2 H), 4.45 (s, 2 H), 3.85 (t, J = 5.2 Hz, 2 H), 3.14 (t, J = 7.2 Hz, 2 H), 2.38 (s, 3 H), 2.10 (m, 5 H). MS calcd. for C₂₃H₂₃F₃NO₄S (M + H⁺) 466.1, found 466.1. |
| C10 | 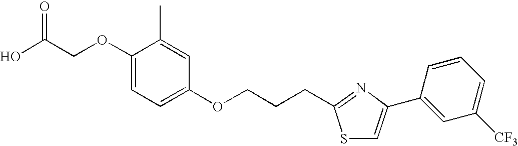 | ¹H-NMR (400 MHz, CDCl₃) δ = 7.84 (s, 1 H), 7.75 (d, J = 7.6 Hz, 1 H), 7.36 (d, J = 7.6 Hz, 1 H), 7.31 (t, J = 7.6 Hz, 1 H), 7.20 (s, 1 H), 6.51 (d, J = 2.4 Hz, 1 H), 6.44 (m, 2 H), 4.38 (s, 2 H), 3.79 (t, J = 6.0 Hz, 2 H), 3.08 (t, J = 6.0 Hz, 2 H), 2.08 (t, J = 6.8 Hz, 2 H), 2.02 (s, 3 H). MS calcd. for C₂₂H₂₁F₃NO₄S (M + H⁺) 452.1, found 452.1. |
| C11 | 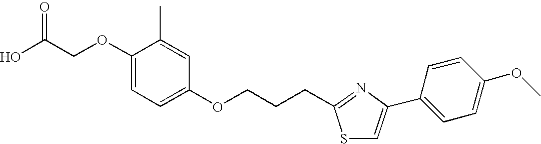 | ¹H-NMR (400 MHz, CDCl₃) δ = 7.83 (d, J = 8.4 Hz, 2 H), 7.24 (s, 1 H), 6.97 (d, J = 8.4 Hz, 2 H), 6.77 (s, 1 H), 6.68 (m, 2 H), 4.62 (s, 2 H), 4.04 (t, J = 5.6 Hz, 2 H), 3.87 (s, 3 H), 3.34 (t, J = 6.8 Hz, 2 H), 2.34 (t, J = 6.8 Hz, 2 H), 2.28 (s, 3 H). MS calcd. for C₂₂H₂₄NO₅S (M + H⁺) 414.1, found 414.2. |
| C12 | 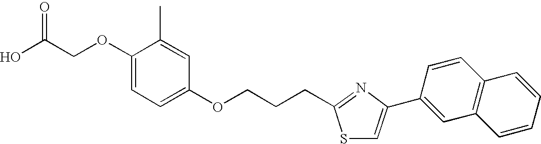 | ¹H-NMR (400 MHz, CDCl₃) δ = 8.24 (s, 1 H), 7.67 (m, 5 H), 7.30 (m, 3 H), 6.57 (s, 1 H), 6.49 (s, 2 H), 4.40 (s, 2 H), 3.86 (t, J = 5.6 Hz, 2 H), 3.19 (t, J = 6.8 Hz, 2 H), 2.17 (t, J = 6.0 Hz, 2 H), 2.06 (s, 3 H). MS calcd. for C₂₅H₂₃NO₄S (M + H⁺) 434.1, found 434.1. |
| C13 | 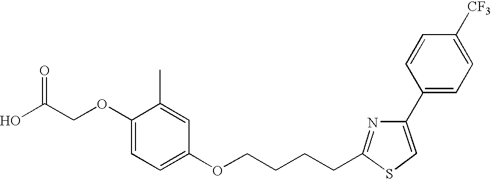 | ¹H-NMR (400 MHz, CDCl₃) δ = 8.01 (d, J = 8.0 Hz, 2 H), 7.69 (d, J = 8.0 Hz, 2 H), 7.48 (s, 1 H), 6.77 (s, 1 H), 6.69 (m, 2 H), 4.63 (s, 2 H), 4.00 (t, J = 6.0 Hz, 2 H), 3.19 (t, J = 7.2 Hz, 2 H), 2.28 (s, 3 H), 2.05 (m, 2 H), 1.94 (m, 2 H). MS calcd. for C₂₃H₂₂F₃NO₄S (M + H⁺) 466.1, found 466.2. |
| C14 | 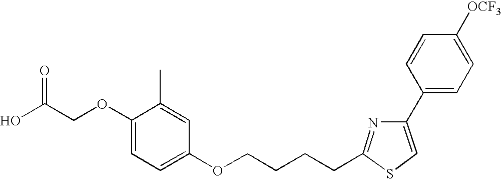 | ¹H-NMR (400 MHz, CDCl₃) δ = 7.65 (d, J = 8.4 Hz, 2 H), 7.10 (s, 1 H), 7.01 (d, J = 8.4 Hz, 2 H), 6.50 (s, 1 H), 6.43 (m, 2 H), 4.36 (s, 2 H), 3.71 (t, J = 6.0 Hz, 2 H), 2.91 (t, J = 7.2 Hz, 2 H), 2.02 (s, 3 H), 1.80 (m, 2 H), 1.67 (m, 2 H). MS calcd. for C₂₃H₂₃F₃NO₅S (M + H⁺) 482.1, found 482.2. |
| C15 | 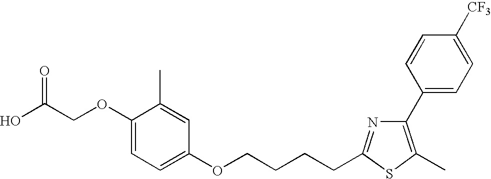 | ¹H-NMR (400 MHz, CDCl₃) δ = 7.73 (d, J = 8.0 Hz, 2 H), 7.67 (d, J = 8.0 Hz, 2 H), 6.73 (s, 1 H), 6.65 (m, 2 H), 4.57 (s, 2 H), 3.93 (t, J = 6.0 Hz, 2 H), 3.08 (t, J = 6.8 Hz, 2 H), 2.53 (s, 3 H), 2.25 (s, 3 H), 1.98 (m, 2 H), 1.88 (m, 2 H). MS calcd. for C₂₄H₂₅F₃NO₄S (M + H⁺) 480.1, found 480.2. |
| C16 | 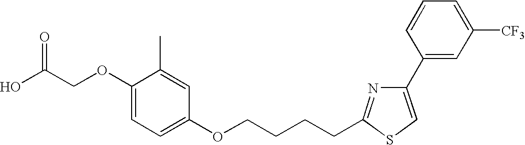 | ¹H-NMR (400 MHz, CDCl₃) δ = 8.12 (s, 1 H), 8.04 (d, J = 7.6 Hz, 1 H), 7.59 (d, J = 7.2 Hz, 1 H), 7.54 (t, J = 8.4 Hz, 1 H), 7.43 (s, 1 H), 6.75 (s, 1 H), 6.66 (m, 2 H), 4.61 (s, 2 H), 3.96 (t, J = 6.0 Hz, 2 H), 3.18 (t, J = 7.2 Hz, 2 H), 2.26 (s, 3 H), 2.04 (m, 2 H), 1.91 (m, 2 H). MS calcd. for C₂₃H₂₃F₃NO₄S (M + H⁺) 466.1, found 466.2. |
| C17 | 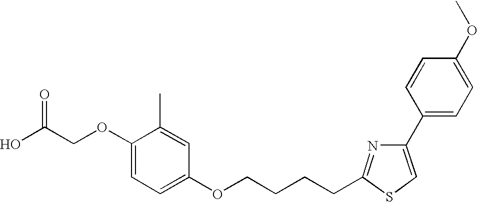 | ¹H-NMR (400 MHz, CDCl₃) δ = 7.59 (d, J = 8.4 Hz, 2 H), 6.99 (s, 1 H), 6.73 (d, J = 8.8 Hz, 2 H), 6.53 (s, 1 H), 6.46 (m, 2 H), 4.38 (s, 2 H), 3.74 (t, J = 6.0 Hz, 2 H), 3.63 (s, 3 H), 2.95 (t, J = 7.6 Hz, 2 H), 2.05 (s, 3 H), 1.80 (m, 2 H), 1.69 (m, 2 H). MS calcd. for C₂₃H₂₆NO₅S (M + H⁺) 428.2, found 428.2. |
| C18 | 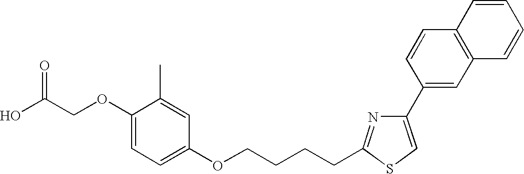 | ¹H-NMR (400 MHz, CDCl₃) δ = 8.36 (s, 1 H), 7.81 (m, 4 H), 7.41 (m, 3 H), 6.69 (s, 1 H), 6.60 (m, 2 H), 4.52 (s, 2 H), 3.91 (t, J = 6.0 Hz, 2 H), 3.16 (t, J = 7.2 Hz, 2 H), 2.19 (s, 3 H), 2.00 (m, 2 H), 1.87 (m, 2 H). MS calcd. for C₂₆H₂₆NO₄S (M + H⁺) 448.2, found 448.2. |
| C19 | 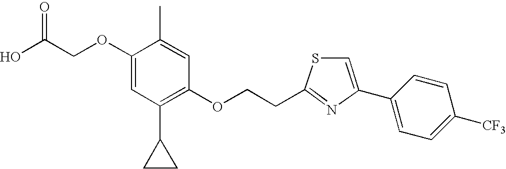 | ¹H-NMR (400 MHz, DMSO) δ = 8.23 (s, 1 H), 8.17 (d, J = 8.8 Hz, 2 H), 7.79 (d, J = 8.8 Hz, 2 H), 6.82 (s, 1 H), 6.25 (s, 1 H), 4.58 (s, 2 H), 4.33 (t, J = 6.0 Hz, 2 H), 3.51 (t, J = 6.0 Hz, 2 H), 2.14 (s, 3 H), 2.06 (m, 1 H), 0.76 (m, 2 H), 0.54 (m, 2 H). MS calcd. for C₂₄H₂₃F₃NO₄S (M + H⁺) 478.1, found 478.1. |
| C20 | 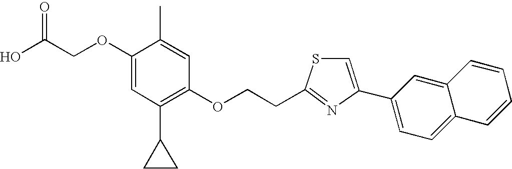 | ¹H-NMR (400 MHz, CDCl₃) δ = 8.41 (s, 1 H), 7.87 (m, 4 H), 7.49 (m, 3 H), 6.72 (s, 1 H), 6.30 (s, 1 H), 4.58 (s, 2 H), 4.40 (t, J = 6.0 Hz, 2 H), 3.64 (t, J = 6.0 Hz, 2 H), 2.24 (s, 3 H), 2.15 (m, 1 H), 0.88 (m, 2 H), 0.56 (m, 2 H). MS calcd. for C₂₇H₂₆NO₄S (M + H⁺) 460.2, found 460.2. |
| C21 | 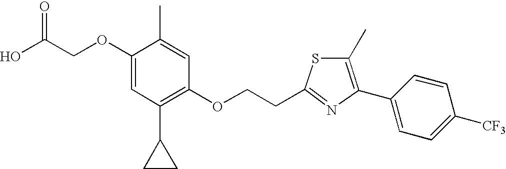 | ¹H-NMR (400 MHz, CDCl₃) δ = 7.51 (d, J = 8.0 Hz, 2 H), 7.44 (d, J = 8.0 Hz, 2 H), 6.45 (s, 1 H), 6.06 (s, 1 H), 4.33 (s, 2 H), 4.08 (t, J = 6.0 Hz, 2 H), 3.27 (t, J = 6.0 Hz, 2 H), 2.30 (s, 3 H), 1.99 (s, 3 H), 1.91 (m, 1 H), 0.65 (m, 2 H), 0.33 (m, 2 H). MS calcd. for C₂₅H₂₅F₃NO₄S (M + H⁺) 492.1, found 492.1. |
| C22 | 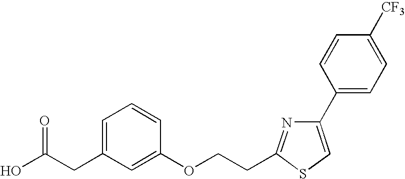 | ¹H-NMR (400 MHz, CDCl₃) δ = 7.75 (d, J = 8.4 Hz, 2 H), 7.43 (d, J = 8.8 Hz, 2 H), 7.03 (m, 2 H), 6.65 (m, 3 H), 4.15 (t, J = 6.4 Hz, 2 H), 3.39 (s, 2 H), 3.32 (t, J = 6.0 Hz, 2 H). MS calcd. for C₂₀H₁₇F₃NO₃S (M + H⁺) 408.1, found 408.1. |
| C23 | 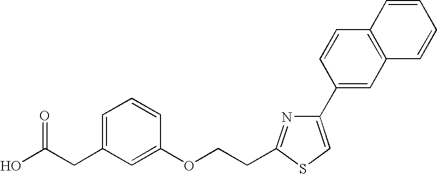 | ¹H-NMR (400 MHz, CDCl₃) δ = 8.58 (s, 1 H), 8.06 (m, 4 H), 7.64 (m, 4 H), 7.04 (m, 3 H), 4.56 (t, J = 6.0 Hz, 2 H), 3.78 (m, 4 H). MS calcd. for C₂₃H₂₀NO₃S (M + H⁺) 390.1, found 390.1. |
| C24 | 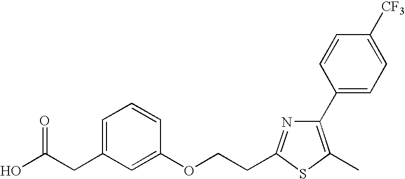 | ¹H-NMR (400 MHz, CDCl₃) δ = 7.52 (d, J = 8.4 Hz, 2 H), 7.47 (d, J = 8.4 Hz, 2 H), 7.04 (m, 1 H), 6.65 (m, 3 H), 4.12 (t, J = 6.0 Hz, 2 H), 3.42 (s, 2 H), 3.32 (t, J = 6.0 Hz, 2 H), 2.32 (s, 3 H). MS calcd. for C₂₁H₁₉F₃NO₃S (M + H⁺) 422.1, found 422.1. |
| C25 | 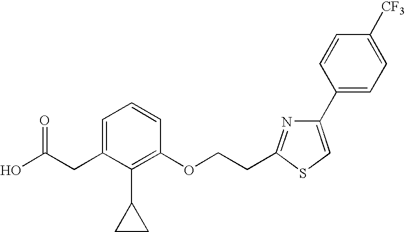 | ¹H-NMR (400 MHz, CDCl₃) δ = 7.88 (d, J = 8.0 Hz, 2 H), 7.55 (d, J = 8.0 Hz, 2 H), 7.38 (s, 1 H), 7.04 (t, J = 8.0 Hz, 1 H), 6.72 (t, J = 8.0 Hz, 2 H), 4.28 (t, J = 6.0 Hz, 2 H), 3.80 (s, 2 H), 3.48 (t, J = 6.0 Hz, 2 H), 1.47 (m, 1 H), 0.80 (m, 2 H), 0.45 (m, 2 H). MS calcd. for C₂₃H₂₁F₃NO₃S (M + H⁺) 448.1, found 448.1. |
| C26 | 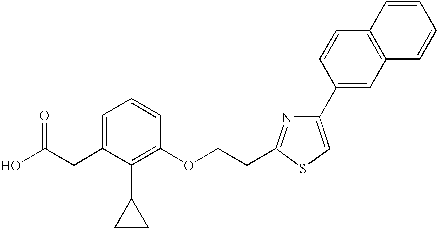 | ¹H-NMR (400 MHz, CDCl₃) δ = 8.35 (s, 1 H), 7.82 (m, 4 H), 7.41 (m, 3 H), 7.08 (t, J = 8.0 Hz, 1 H), 6.76 (d, J = 8.0 Hz, 2 H), 4.34 (t, J = 6.0 Hz, 2 H), 3.84 (s, 2 H), 3.55 (t, J = 6.0 Hz, 2 H), 1.51 (m, 1 H), 0.87 (m, 2 H), 0.50 (m, 2 H). MS calcd. for C₂₆H₂₄NO₃S (M + H⁺) 430.1, found 430.1. |
| C27 | 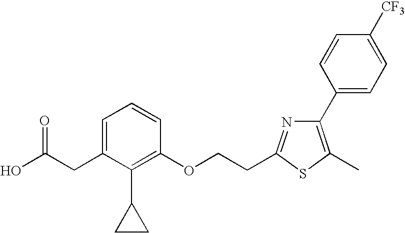 | ¹H-NMR (400 MHz, CDCl₃) δ = 7.76 (d, J = 8.0 Hz, 2 H), 7.69 (d, J = 8.0 Hz, 2 H), 7.15 (t, J = 8.0 Hz, 1 H), 6.83 (m, 2 H), 4.34 (t, J = 6.0 Hz, 2 H), 3.93 (s, 2 H), 3.54 (t, J = 6.0 Hz, 2 H), 2.55 (s, 3 H), 1.61 (m, 1 H), 0.96 (m, 2 H), 0.60 (m, 2 H). MS calcd. for C₂₄H₂₃F₃NO₃S (M + H⁺) 462.1, found 462.1. |
| C28 | 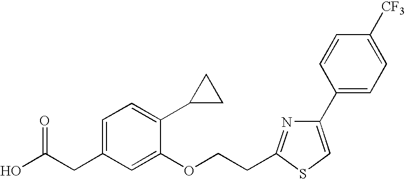 | ¹H-NMR (400 MHz, CDCl₃) δ = 7.93 (d, J = 8.4 Hz, 2 H), 7.61 (d, J = 8.4 Hz, 2 H), 7.43 (s, 1 H), 6.74 (m, 3 H), 4.36 (t, J = 6.0 Hz, 2 H), 3.55 (t, J = 6.0 Hz, 2 H), 3.53 (s, 2 H), 2.10 (m, 1 H), 0.82 (m, 2 H), 0.55 (m, 2 H). MS calcd. for C₂₃H₂₁F₃NO₃S (M + H⁺) 448.1, found 448.1. |
| C29 | 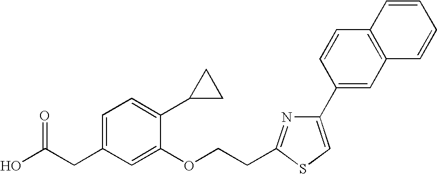 | ¹H-NMR (400 MHz, CDCl₃) δ = 8.38 (s, 1 H), 7.85 (m, 4 H), 7.45 (m, 3 H), 6.76 (m, 3 H), 4.40 (t, J = 6.0 Hz, 2 H), 3.62 (t, J = 6.0 Hz, 2 H), 3.56 (s, 2 H), 2.15 (m, 1 H), 0.85 (m, 2 H), 0.58 (m, 2 H). MS calcd. for C₂₆H₂₄NO₃S (M + H⁺) 430.1, found 430.1. |
| C30 | 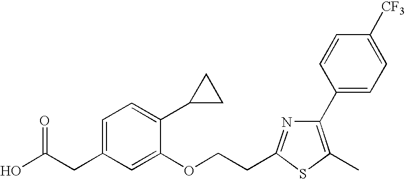 | ¹H-NMR (400 MHz, CDCl₃) δ = 7.75 (d, J = 8.0 Hz, 2 H), 7.68 (d, J = 8.0 Hz, 2 H), 6.78 (m, 3 H), 4.36 (t, J = 6.0 Hz, 2 H), 3.58 (s, 2 H), 3.55 (t, J = 6.0 Hz, 2 H), 2.18 (m, 1 H), 0.89 (m, 2 H), 0.62 (m, 2 H). MS calcd. for C₂₄H₂₃F₃NO₃S (M + H⁺) 462.1, found 462.1. |
| C31 | 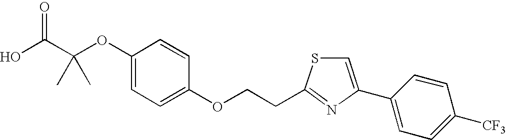 | ¹H-NMR (400 MHz, CDCl₃) δ = 7.99 (d, J = 8.4 Hz, 2 H), 7.66 (d, J = 8.4 Hz, 2 H), 7.49 (s, 1 H), 6.92 (d, J = 8.8 Hz, 2 H), 6.87 (d, J = 8.8 Hz, 2 H), 4.36 (t, J = 5.6 Hz, 2 H), 3.54 (t, J = 6.0 Hz, 2 H), 1.54 (s, 6 H). MS calcd. for C₂₂H₂₁F₃NO₄S (M + H⁺) 452.1, found 452.1. |
| C32 | 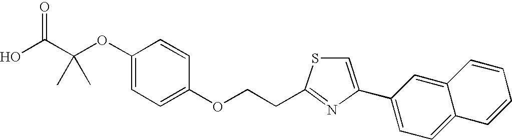 | ¹H-NMR (400 MHz, CDCl₃) δ = 8.42 (s, 1 H), 7.90 (m, 4 H), 7.49 (m, 3 H), 7.27 (s, 1 H), 6.93 (d, J = 8.8 Hz, 2 H), 6.88 (d, J = 8.8 Hz, 2 H), 4.39 (t, J = 5.6 Hz, 2 H), 3.59 (t, J = 6.0 Hz, 2 H), 1.55 (s, 6 H). MS calcd. for C₂₅H₂₄NO₄S (M + H⁺) 434.1, found 434.1. |
| C33 | 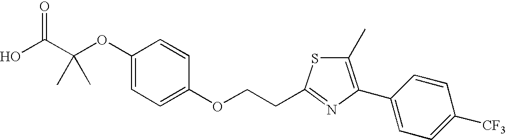 | ¹H-NMR (400 MHz, CDCl₃) δ = 7.74 (d, J = 8.4 Hz, 2 H), 7.68 (d, J = 8.4 Hz, 2 H), 6.92 (d, J = 8.8 Hz, 2 H), 6.85 (d, J = 8.8 Hz, 2 H), 4.30 (t, J = 6.0 Hz, 2 H), 3.46 (t, J = 6.0 Hz, 2 H), 2.55 (s, 3 H), 1.53 (s, 6 H). MS calcd. for C₂₃H₂₃F₃NO₄S (M + H⁺) 466.1, found 466.1. |

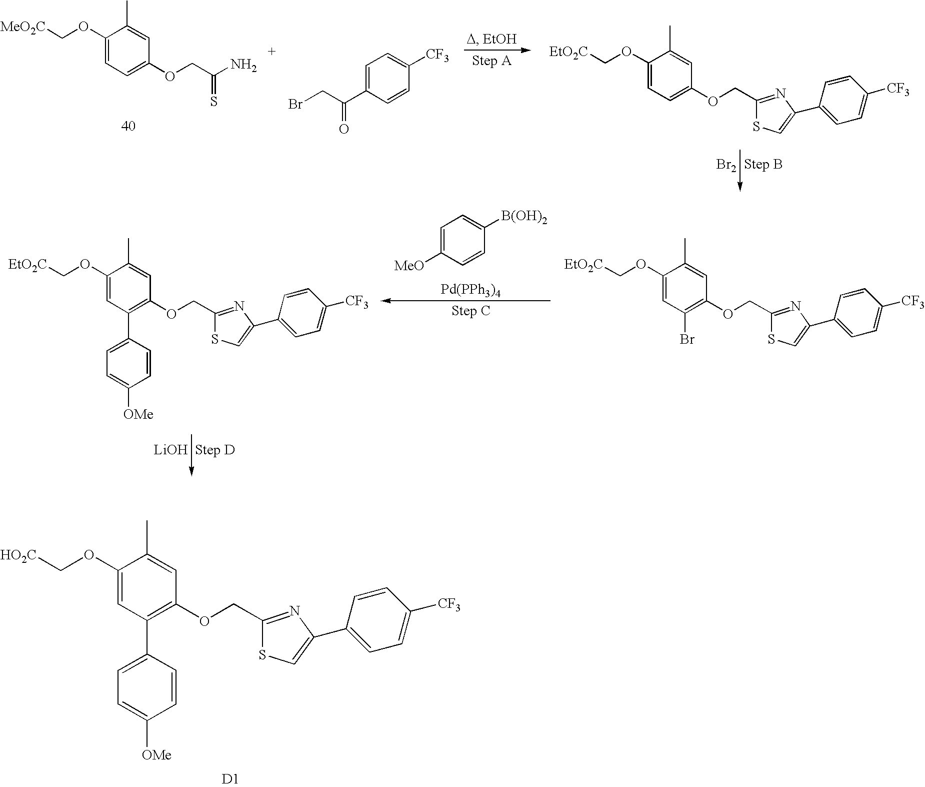

### Example D1

### {4′-Methoxy-4-methyl-6-[4-(4-trifluoromethylphenyl)-thiazol-2-ylmethoxy]-biphenyl-3-yloxy}-acetic Acid

Step A: (2-Methyl-4-thiocarbamoylmethoxy-phenoxy)-acetic acid methyl ester 40 (315 mg, 1.17 mmol) and 2-bromo-1-(4-trifluoromethyl-phenyl)-ethanone (298 mg, 1.11 mmol) are dissolved in EtOH (5 mL) and heated at reflux for 2 h. The mixture is cooled and the resulting precipitate is collected by filtration, washed with MeOH, and dried under vacuum to give {2-Methyl-4-[4-(4-trifluoromethyl-phenyl)-thiazol-2-ylmethoxy]-phenoxy}-acetic acid ethyl ester: ¹H-NMR (400 MHz, CDCl₃) 8.01 (d, J=8.0 Hz, 2H), 7.68 (d, J=8.0 Hz, 2H), (s, 1H), 6.89 (d, J=2.4 Hz, 1H), 6.76 (dd, J=2.8, 8.4 Hz, 1H), 6.67 (d, J=8.8 Hz, 1H), 5.37 (s, 2H), 4.59 (s, 2H), (q, J=7.2 Hz, 2H), 2.29 (s, 3H), 1.29 (t, J=7.2 Hz, 3H). MS calcd. for C₂₂H₂₁F₃NO₄S (M+H⁺) 452.1, found 452.4.

Step B: {2-Methyl-4-[4-(4-trifluoromethyl-phenyl)-thiazol-2-ylmethoxy]-phenoxy}-acetic acid ethyl ester (220 mg, 0.48 mmol) is dissolved in dichloromethane (3 mL). Bromine (62 μL, 1.2 mmol) is predissolved in AcOH (0.2 mL), then is added to the solution. The mixture is stirred at rt for 3 h, then is poured into water (10 mL) and extracted with dichloromethane (10 mL). The organic layer is washed successively with saturated solutions of NaHCO₃ and sodium bisulfite, dried (MgSO₄), filtered and concentrated. The residue is triturated with hexanes and filtered to afford {5-bromo-2-methyl-4-[4-(4-trifluoromethyl-phenyl)-thiazol-2-ylmethoxy]-phenoxy}-acetic acid ethyl ester: ¹H-NMR (400 MHz, CDCl₃) 7.95 (d, J=8.0 Hz, 2H), 7.62 (d, J=8.0 Hz, 2H), 7.58 (s, 1H), 6.88 (s, 1H), 6.83 (s, 1H), 5.37 (s, 2H), 4.53 (s, 2H), 4.20 (q, J=7.2 Hz, 2H), 2.19 (s, 3H), 1.24 (t, J=7.2 Hz, 3H). MS calcd. for C₂₂H₂₀BrF₃NO₄S (M+H⁺) 530.0, found 530.2.

Step C: {5-Bromo-2-methyl-4-[4-(4-trifluoromethyl-phenyl)-thiazol-2-ylmethoxy]-phenoxy}-acetic acid ethyl ester (20 mg, 0.04 mmol), 4-methoxyphenylboronic acid (7.2 mg, 0.05 mmol) and sodium carbonate (13 mg, 0.12 mmol) are dissolved in a mixture of water (120 μL), ethanol (90 μL) and 1,2-dimethoxyethane (360 μL). The mixture is degassed with Argon for 2 minutes. Pd(PPh₃)₄ (10 mol %) is added and the mixture is subjected to microwave irradiation (180° C.) for 5 min in a sealed tube to give crude {4′-methoxy-4-methyl-6-[4-(4-trifluoromethyl-phenyl)-thiazol-2-ylmethoxy]-biphenyl-3-yloxy}-acetic acid ethyl ester, which is used without further purification in Step B. MS calcd. for C₂₉H₂₇F₃NO₅S (M+H⁺) 558.2, found 558.3

Step D: To the solution of {4-[4-(4-methoxy-phenyl)-5-(4-trifluoromethoxy-phenyl)-thiazol-2-ylmethoxy]-2-methyl-phenoxy}-acetic acid methyl ester from Step C is added THF (1 mL) and a solution of 1 M LiOH in H₂O (0.2 mL). The mixture is stirred for 1 h at rt, then it is acidified with 1 M HCl (0.3 mL). EtOAc (20 mL) is added and the organic layer is washed with brine (10 mL). The organic layer is dried (MgSO₄), filtered, concentrated and purified on reverse phase HPLC (H₂O/MeCN gradient) to afford the title compound D1 as a white solid: ¹H-NMR (400 MHz, CDCl₃) δ=7.97 (d, J=8.0 Hz, 2H), (d, J=8.0 Hz, 2H), 7.55 (5, 1H), 7.49 (d, J=8.4 Hz, 2H), 6.98 (s, 1H), 6.94 (d, J=8.4 Hz, 2H), 6.78 (s, 1H), (5, 2H), 4.69 (s, 2H), 3.85 (s, 3H), 2.33 (s, 3H). MS calcd. for C₂₇H₂₃F₃NO₅S (M+H⁺) 530.1, found 530.3.

By repeating the procedures described in the above examples, using appropriate starting materials, the following compounds of Formula I, as identified in Table 4, are obtained.

TABLE 4

| | | Physical Data |
|---|---|---|
| Compound | Compound | ¹H NMR 400 MHz (DMSO-d₆) |
| Number | Structure | and/or MS (m/z) |
| D2 | 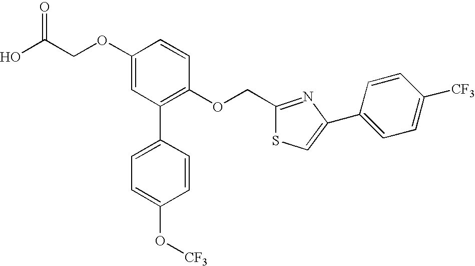 | ¹H-NMR (400 MHz, CDCl₃) δ = 7.97 (d, J = 8.0 Hz, 2 H), 7.67 (d, J = 8.0 Hz, 2 H), 7.56 (m, 3 H), 7.27 (d, J = 8.8 Hz, 2 H), 6.96 (s, 1 H), 6.77 (s, 1 H), 5.32 (s, 2 H), 4.70 (s, 2 H), 2.35 (s, 3 H). MS calcd. for C₂₇H₁₉F₆NO₅S (M + H⁺) 584.1, found 584.2. |
| D3 | 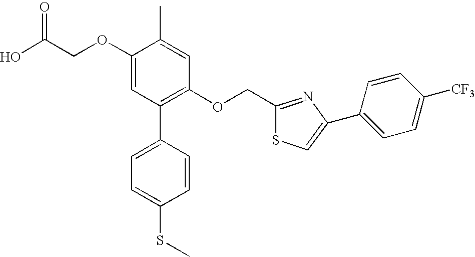 | ¹H-NMR (400 MHz, CDCl₃) δ = 7.97 (d, J = 8.0 Hz, 2 H), 7.67 (d, J = 8.4 Hz, 2 H), 7.56 (s, 1 H), 7.49 (d, J = 8.4 Hz, 2 H), 7.31 (d, J = 8.0 Hz, 2 H), 6.95 (s, 1 H), 6.78 (s, 1 H), 5.31 (s, 2 H), 4.69 (s, 2 H), 2.53 (s, 3 H), 2.33 (s, 3 H). MS calcd. for C₂₇H₂₃F₃NO₄S₂ (M + H⁺) 546.1, found 546.3. |
| D4 | 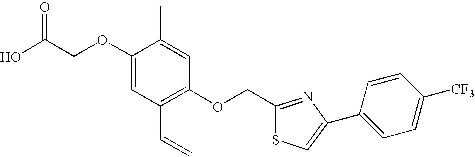 | ¹H-NMR (400 MHz, CDCl₃) δ = 8.01 (d, J = 8.0 Hz, 2 H), 7.68 (d, J = 8.4 Hz, 2 H), 7.62 (s, 1 H), 6.94 (s, 1 H), 6.83 (s, 1 H), 5.70 (d, J = 17.6 Hz, 1 H), 5.38 (s, 2 H), 5.30 (d, J = 11.2 Hz, 1 H), 4.68 (s, 2 H), 2.29 (s, 3 H). MS calcd. for C₂₂H₁₉F₃NO₄S (M + H⁺) 450.1, found 450.1 |
| D5 | 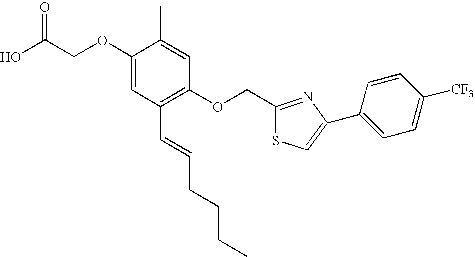 | ¹H-NMR (400 MHz, CDCl₃) δ = 8.01 (d, J = 8.0 Hz, 2 H), 7.68 (d, J = 8.4 Hz, 2 H), 7.62 (s, 1 H), 6.88 (s, 1 H), 6.81 (s, 1 H), 5.72 (d, J = 16.0 Hz, 1 H), 5.38 (s, 2 H), 6.19 (dt, J = 7.2, 15.6 Hz, 1 H), 5.37 (s, 2 H), 4.68 (s, 2 H), 2.27 (s, 3 H), 2.24 (m, 2 H), 1.46 (m, 2 H), 1.37 (m, 2 H), 0.92 (t, J = 7.2 Hz, 3 H). MS calcd. for C₂₆H₂₇F₃NO₄S (M + H⁺) 506.1, found 506.2. |
| D6 | 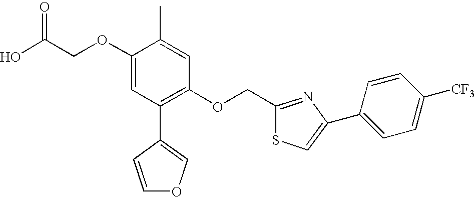 | ¹H-NMR (400 MHz, CDCl₃) δ = 8.03 (s, 1 H), 8.02 (d, J = 8.4 Hz, 2 H), 7.69 (d, J = 8.4 Hz, 2 H), 7.62 (s, 1 H), 7.48 (s, 1 H), 6.94 (s, 1 H), 6.93 (s, 1 H), 6.75 (s, 1 H), 5.44 (s, 2 H), 4.70 (s, 2 H), 2.32 (s, 3 H). MS calcd. for C₂₄H₁₉F₃NO₅S (M + H⁺) 490.1, found 490.0. |
| D7 | 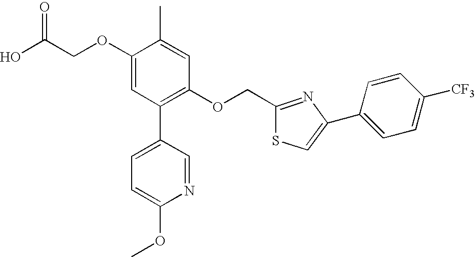 | ¹H-NMR (400 MHz, CDCl₃) δ = 8.38 (d, J = 2.0 Hz, 1 H), 8.00 (d, J = 2.4 Hz, 1 H), 7.96 (d, J = 8.0 Hz, 2 H), 7.67 (d, J = 8.0 Hz, 2 H), 7.54 (s, 1 H), 6.95 (s, 1 H), 6.87 (d, J = 8.4 Hz, 1 H), 6.76 (s, 1 H), 5.32 (s, 2 H), 4.69 (s, 2 H), 2.33 (s, 3 H). MS calcd. for C₂₆H₂₂F₃N₂O₃S (M + H⁺) 531.1, found 531.1. |
| D8 | 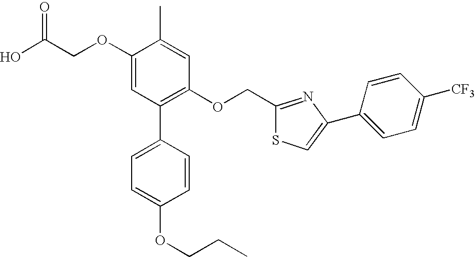 | ¹H-NMR (400 MHz, CDCl₃) δ = 7.97 (d, J = 8.0 Hz, 2 H), 7.67 (d, J = 8.0 Hz, 2 H), 7.55 (s, 1 H), 7.48 (d, J = 8.8 Hz, 2 H), 6.96 (d, J = 8.8 Hz, 2 H), 6.93 (s, 1 H), 6.78 (s, 1 H), 5.29 (s, 2 H), 4.69 (s, 2 H), 3.96 (t, J = 6.8 Hz, 2 H), 2.33 (s, 3 H), 1.83 (m, 2 H), 1.06 (t, J = 7.2 Hz, 3 H). MS calcd. for C₂₉H₂₇F₃NO₅S (M + H⁺) 558.1, found 558.0. |
| D9 | 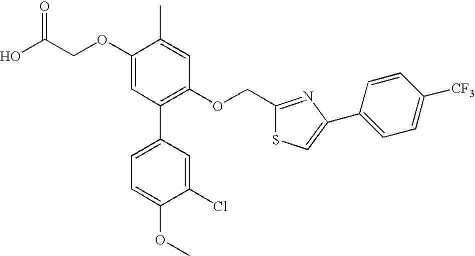 | ¹H-NMR (400 MHz, DMSO- d6) δ = 8.40 (s, 1 H), 8.20 (d, J = 8.4 Hz, 2 H), 7.84 (d, J = 8.4 Hz, 2 H), 7.70 (d, J = 2.4 Hz, 1 H), 7.52 (dd, J = 2.4, 8.4 Hz, 1 H), 7.23 (d, J = 8.8 Hz, 2 H), 7.21 (s, 1 H), 6.86 (s, 1 H), 5.47 (s, 2 H), 4.74 (s, 2 H), 3.92 (s, 3 H), 2.28 (s, 3 H). MS calcd. for C₂₇H₂₂ClF₃NO₅S (M + H⁺) 564.1, found 563.9. |
| D10 | 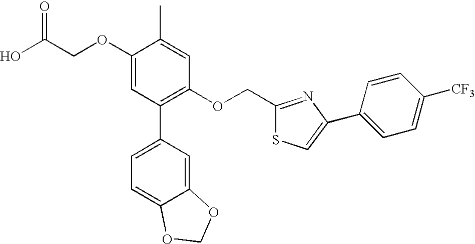 | ¹H-NMR (400 MHz, CDCl₃) δ = 7.96 (d, J = 8.4 Hz, 2 H), 7.67 (d, J = 8.4 Hz, 2 H), 7.56 (s, 1 H), 7.07 (d, J = 1.6 Hz, 1 H), 7.00 (dd, J = 1.6, 8.0 Hz, 1 H), 6.93 (s, 1 H), 6.87 (d, J = 8.0 Hz, 1 H), 6.75 (s, 1 H), 5.30 (s, 2 H), 4.69 (s, 2 H), 2.32 (s, 3 H). MS calcd. for C₂₇H₂₁F₃NO₆S (M + H⁺) 544.1, found 543.9. |
| D11 | 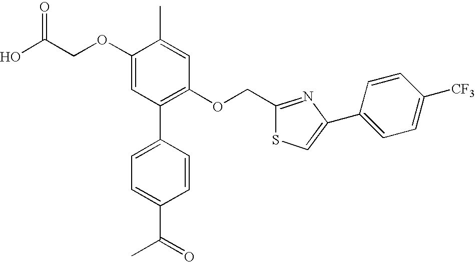 | ¹H-NMR (400 MHz, DMSO- d6) δ = 8.32 (s, 1 H), 8.15 (d, J = 8.0 Hz, 2 H), 8.01 (d, J = 8.4 Hz, 2 H), 7.80 (d, J = 8.4 Hz, 2 H), 7.73 (d, J = 8.4 Hz, 2 H), 7.23 (s, 1 H), 6.89 (s, 1 H), 5.47 (s, 2 H), 4.75 (s, 2 H), 2.61 (s, 3 H), 2.27 (s, 3 H). MS calcd. for C₂₈H₂₃F₃NO₅S (M + H⁺) 542.1, found 542.0. |
| D12 | 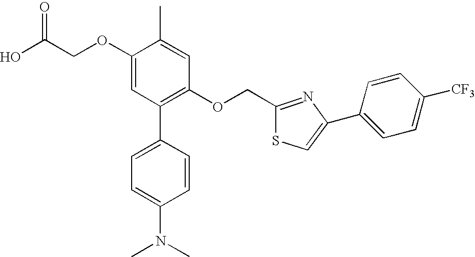 | ¹H-NMR (400 MHz, DMSO- d6) δ = 8.35 (s, 1 H), 8.17 (d, J = 8.4 Hz, 2 H), 7.82 (d, J = 8.4 Hz, 2 H), 7.46 (d, J = 8.4 Hz, 2 H), 7.12 (s, 1 H), 6.76 (s, 1 H), 5.40 (s, 2 H), 4.72 (s, 2 H), 2.96 (s, 6 H), 2.23 (s, 3 H). MS calcd. for C₂₈H₂₆F₃N₂O₄S (M + H⁺) 543.2, found 543.0. |
| D13 | 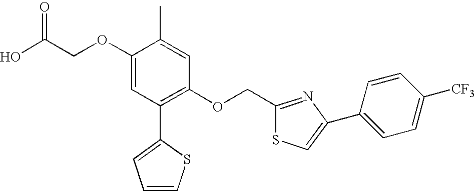 | ¹H-NMR (400 MHz, CDCl₃) δ = 7.93 (d, J = 8.4 Hz, 2 H), 7.61 (d, J = 8.4 Hz, 2 H), 7.36 (s, 1 H), 7.39 (dd, J = 1.2, 8.0 Hz, 1 H), 7.27 (dd, J = 1.2, 8.8 Hz, 1 H), 7.03 (m, 1 H), 6.97 (s, 1 H), 6.89 (s, 1 H), 5.38 (s, 2 H), 4.64 (s, 2 H), 2.24 (s, 3 H). MS calcd. for C₂₄H₁₉F₃NO₄S₂ (M + H⁺) 506.1, found 506.0. |
| D14 | 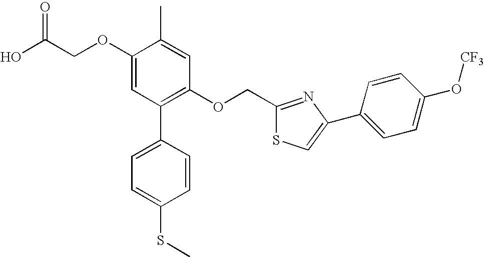 | ¹H-NMR (400 MHz, CDCl₃) δ 8.10 (d, J = 8.4 Hz, 2 H), 7.70 (d, J = 8.4 Hz, 2 H), 7.66 (s, 1 H), 7.52 (d, J = 8.0 Hz, 2 H), 7.48 (d, J = 8.0 Hz, 2 H), 7.16 (s, 1 H), 6.99 (s, 1 H), 5.52 (s, 2 H), 4.90 (s, 2 H), 2.74 (s, 3 H), 2.55 (s, 3 H). MS calcd. for C₂₇H₂₃F₃NO₅S₂ (M + H⁺) 562.1, found 562.2. |
| D15 | 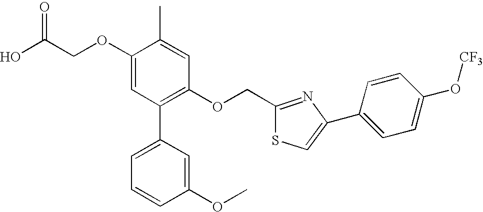 | ¹H-NMR (400 MHz, CDCl₃) δ = 7.91 (d, J = 8.4 Hz, 2 H), 7.46 (s, 1 H), 7.38 (t, J = 8.0 Hz, 1 H), 7.29 (d, J = 8.4 Hz, 2 H), 7.16 (d, J = 8.0 Hz, 1 H), 7.14 (s, 1 H), 6.98 (s, 1 H), 6.94 (dd, J = 2.8, 8.0 Hz, 1 H), 6.84 (s, 1 H), 5.32 (s, 2 H), 4.72 (s, 2 H), 3.86 (s, 3 H), 2.37 (s, 3 H). MS calcd. for C₂₇H₂₃F₃NO₆S (M + H⁺) 546.1, found 546.3. |
| D16 | 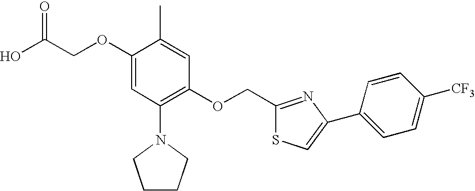 | ¹H-NMR (400 MHz, CDCl₃) δ = 7.93 (d, J = 8.0 Hz, 2 H), 7.64 (d, J = 8.0 Hz, 2 H), 7.61 (s, 1 H), 7.35 (s, 1 H), 6.96 (s, 1 H), 5.43 (s, 2 H), 4.61 (s, 2 H), 3.75 (s, 4 H), 2.23 (s, 3 H), 2.12 (s, 4 H). MS calcd. for C₂₇H₂₃F₃NO₆S (M + H⁺) 493.1, found 493.1. |
| D17 | 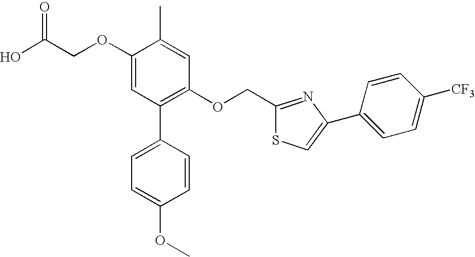 | MS calcd. for C₂₈H₂₅F₃NO₅S (M + H⁺) 544.1, found 544.1. |
| D18 | 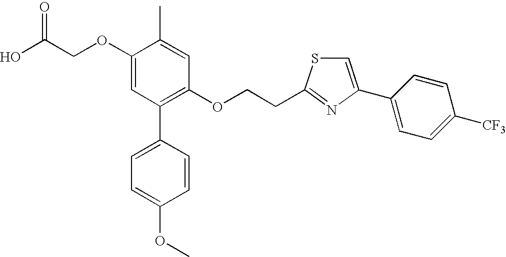 | ¹H-NMR (400 MHz, DMSO- d6) δ = 8.34 (s, 1 H), 8.17 (d, J = 8.0 Hz, 2 H), 7.80 (d, J = 8.4 Hz, 2 H), 7.25 (d, J = 8.8 Hz, 2 H), 6.99 (s, 1 H), 6.75 (d, J = 8.8 Hz, 2 H), 6.71 (s, 1 H), 4.68 (s, 2 H), 4.34 (t, J = 6.0 Hz, 2 H), 3.63 (s, 3 H), 3.42 (t, J = 6.0 Hz, 2 H), 2.22 (s, 3 H). MS calcd. for C₂₈H₂₅F₃NO₅S (M + H⁺) 544.1, found 544.1. |
| D19 | 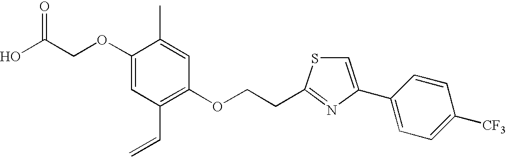 | ¹H-NMR (400 MHz, DMSO- D6) δ = 8.16 (s, 1 H), 8.09 (d, J = 8.0 Hz, 2 H), 7.72 (d, J = 8.4 Hz, 2 H), 6.90 (s, 1 H), 6.84 (s, 1 H), 6.79 (dd, J = 12.0, 18.0 Hz, 1 H), 5.63 (d, J = 16.4 Hz, 1 H), 5.04 (d, J = 12.0 Hz, 1 H), 4.60 (s, 2 H), 4.26 (t, J = 6.0 Hz, 2 H), 3.44 (t, J = 6.0 Hz, 2 H), 2.10 (s, 3 H). MS calcd. for C₂₃H₂₁F₃NO₄S (M + H⁺) 464.1, found 464.1. |

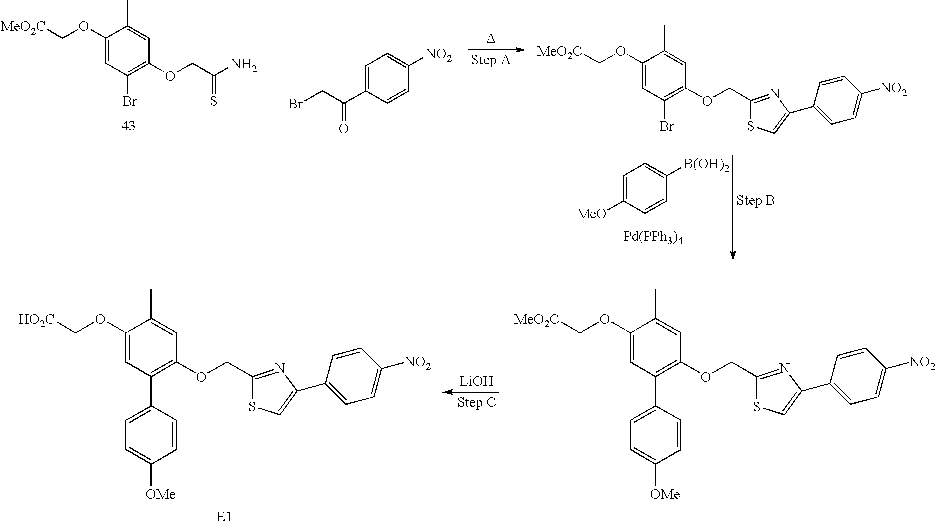

### Example E1

### {4′-Methoxy-4-methyl-6-[4-(4-nitro-phenyl)-thiazol-2-ylmethoxy]-biphenyl-3-yloxy}-acetic Acid

Step A: (5-Bromo-2-methyl-4-thiocarbamoylmethoxy-phenoxy)-acetic acid methyl ester 43 (50 mg, 0.14 mmol) and 2-bromo-1-(4-nitro-phenyl)-ethanone (40 mg, 0.17 mmol) are dissolved in EtOH (1 mL) and heated at reflux for 2 h. The mixture is cooled, the resulting precipitate is filtered and washed to afford {5-bromo-2-methyl-4-[4-(4-nitro-phenyl)-thiazol-2-ylmethoxy]-phenoxy}-acetic acid ethyl ester as a white powder which is used in Step B without further purification. MS calcd. for C₂₁H₂₀BrN₂O₆S (M+H⁺) 507.1, found 506.9.

Step B: {5-Bromo-2-methyl-4-[4-(4-nitro-phenyl)-thiazol-2-ylmethoxy]-phenoxy}-acetic acid ethyl ester (26 mg, 0.053 mmol), 4-methoxyphenylboronic acid (8 mg, 0.053 mmol) and sodium carbonate (17 mg, 0.16 mmol) are dissolved in a mixture of water (120 μL), ethanol (90 μL) and 1,2-dimethoxyethane (360 μL). The mixture is degassed with Argon for 2 minutes. Pd(PPh₃)₄ (10 mol %) is added and the mixture is subjected to microwave irradiation (180° C.) for 5 min in a sealed tube to give crude {4′-methoxy-4-methyl-6-[4-(4-nitro-phenyl)-thiazol-2-ylmethoxy]-biphenyl-3-yloxy}-acetic acid ethyl ester, which is used directly in Step C. MS calcd. for C₂₈H₂₇N₂O₇S (M+H⁺) 535.2, found 535.1.

Step C: To the solution of {4′-methoxy-4-methyl-6-[4-(4-nitrophenyl)-thiazol-2-ylmethoxy]-biphenyl-3-yloxy}-acetic acid ethyl ester from Step A is added THF (1 mL) and a solution of 1 M LiOH in H₂O (0.2 mL). The mixture is stirred for 1 h at rt. Then it is acidified with 1 M HCl (0.25 mL), EtOAc (10 mL) is added and the organic layer is washed with brine. The organic layer is dried (MgSO₄), filtered, concentrated and purified on reverse phase HPLC (H₂O/MeCN gradient) to afford the title compound E1 as a white solid: ¹H-NMR (400 MHz, DMSO-d6) δ=8.21 (s, 1H), 8.07 (d, J=8.8 Hz, 2H), 7.97 (d, J=8.8 Hz, 2H), 7.26 (d, J=8.4 Hz, 2H), 6.91 (s, 1H), 6.75 (d, J=8.8 Hz, 2H), 6.55 (s, 1H), 5.18 (s, 2H), 4.48 (s, 2H), 3.55 (s, 3H), 1.99 (s, 3H). MS calcd. for C₂₆H₂₃N₂O₇S (M+H⁺) 507.1, found 507.1.

By repeating the procedures described in the above examples, using appropriate starting materials, the following compounds of Formula I, as identified in Table 5, are obtained.

TABLE 5

| | | Physical Data |
|---|---|---|
| Compound | Compound | ¹H NMR 400 MHz (DMSO-d₆) |
| Number | Structure | and/or MS (m/z) |
| E2 | 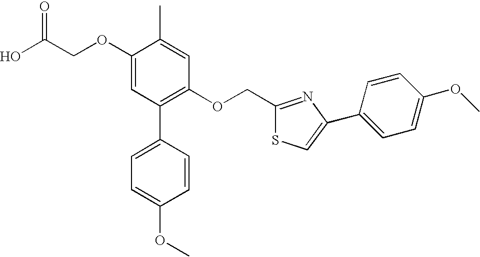 | ¹H-NMR (400 MHz, DMSO- d6) δ = 7.92 (s, 1 H), 7.87 (d, J = 8.8 Hz, 2 H), 7.51 (d, J = 8.8 Hz, 2 H), 7.14 (s, 1 H), 7.00 (m, 4 H), 6.79 (s, 1 H), 5.38 (s, 2 H), 4.72 (s, 2 H), 3.80 (s, 6 H), 2.24 (s, 3 H). MS calcd. for C₂₇H₂₆NO₆S (M + H⁺) 492.1, found 492.2. |
| E3 | 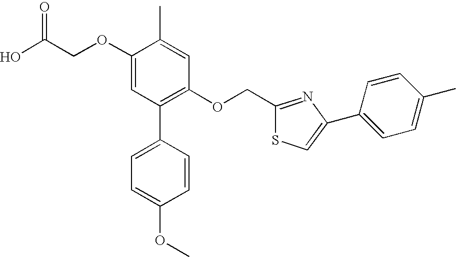 | ¹H-NMR (400 MHz, DMSO- d6) δ = 8.01 (s, 1 H), 7.83 (d, J = 8.4 Hz, 2 H), 7.51 (d, J = 8.8 Hz, 2 H), 7.25 (d, J = 8.0 Hz, 2 H), 7.15 (s, 1 H), 6.99 (d, J = 8.8 Hz, 2 H), 6.79 (s, 1 H), 5.38 (s, 2 H), 4.72 (s, 2 H), 3.79 (s, 3 H), 2.33 (s, 3 H), 2.24 (s, 3 H). MS calcd. for C₂₇H₂₆NO₅S (M + H⁺) 476.1, found 476.1. |
| E4 | 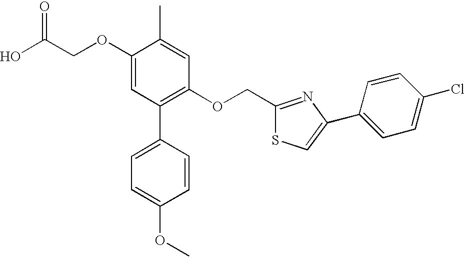 | ¹H-NMR (400 MHz, DMSO- d6) δ = 8.16 (s, 1 H), 7.96 (d, J = 8.4 Hz, 2 H), 7.51 (m, 4 H), 7.14 (s, 1 H), 6.99 (d, J = 8.8 Hz, 2 H), 6.79 (s, 1 H), 5.39 (s, 2 H), 4.72 (s, 2 H), 3.79 (s, 3 H), 2.24 (s, 3 H). MS calcd. for C₂₆H₂₃ClNO₅S (M + H⁺) 496.1, found 496.0. |
| E5 | 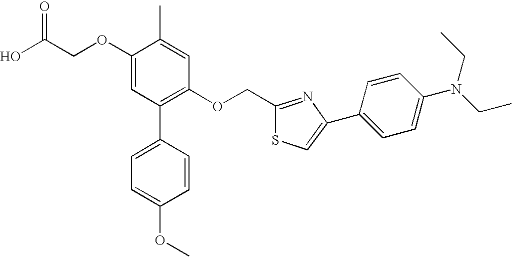 | ¹H-NMR (400 MHz, CDCl₃) δ = 7.98 (d, J = 8.4 Hz, 2H), 7.56 (d, J = 8.4 Hz, 2 H), 7.50 (s, 1 H), 6.45 (d, J = 8.8 Hz, 2 H), 6.93 (m, 3 H), 6.76 (s, 1 H), 5.25 (s, 2 H), 4.68 (s, 2 H), 3.51 (m, 4 H), 2.32 (s, 2 H), 1.14 (t, J = 7.2 Hz, 6 H). MS calcd. for C₂₆H₂₃ClNO₅S (M + H⁺) 533.2, found 533.2. |
| E6 | 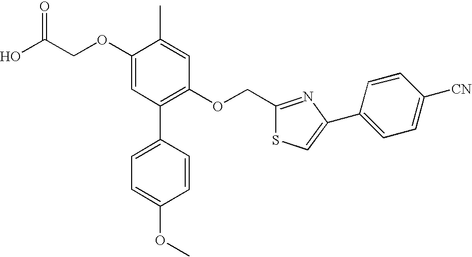 | ¹H-NMR (400 MHz, CDCl₃) δ = 7.90 (d, J = 8.4 Hz, 2 H), 7.63 (d, J = 8.8 Hz, 2 H), 7.51 (s, 1 H), 7.42 (d, J = 8.8 Hz, 2 H), 6.90 (d, J = 8.8 Hz, 2 H), 6.87 (s, 1 H), 6.72 (s, 1 H), 5.21 (s, 2 H), 4.62 (s, 2 H), 3.78 (s, 3 H), 2.26 (s, 3 H). MS calcd. for C₂₇H₂₃N₂O₅S (M + H⁺) 487.1, found 487.0. |
| E7 | 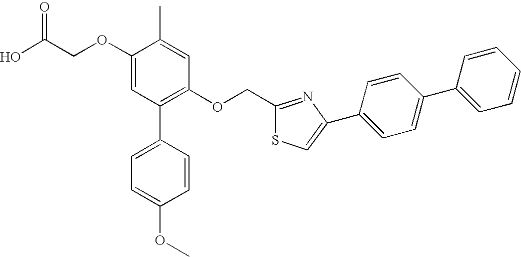 | MS calcd. for C₃₂H₂₈NO₅S (M + H⁺) 538.2, found 538.1. |
| E8 | 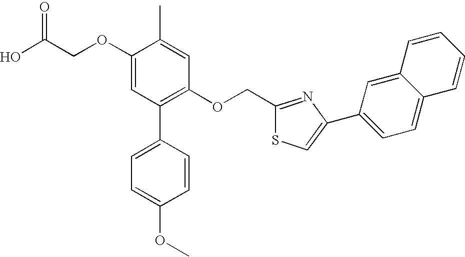 | ¹H-NMR (400 MHz, DMSO- d6) δ = 8.52 (s, 1 H), 8.25 (s, 1 H), 8.09 (d, J = 8.8 Hz, 1 H), 8.00 (m, 2 H), 7.94 (m, 1 H), 7.53 (d, J = 8.8 Hz, 4 H), 7.18 (s, 1 H), 7.00 (d, J = 8.8 Hz, 2 H), 6.80 (s, 1 H), 5.44 (s, 2 H), 4.73 (s, 2 H), 3.79 (s, 3 H), 2.25 (s, 3 H). MS calcd. for C₃₀H₂₆NO₅S (M + H⁺) 512.1, found 512.1. |
| E9 | 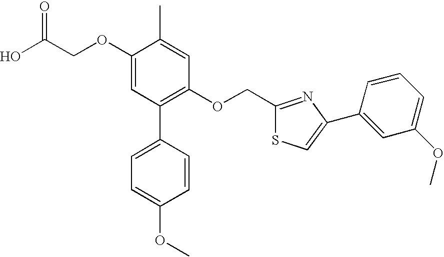 | ¹H-NMR (400 MHz, CDCl₃) δ = 7.49 (d, J = 8.8 Hz, 2 H), 7.43 (m, 3 H), 7.33 (t, J = 8.0 Hz, 1 H), 6.97 (m, 3 H), 6.90 (dd, J = 2.0, 7.6 Hz, 1 H), 6.78 (s, 1 H), 5.32 (s, 2 H). 4.68 (s, 2 H), 3.88 (s, 3 H), 3.85 (s, 3 H), 2.33 (s, 3 H). MS calcd. for C₂₇H₂₆NO₆S (M + H⁺) 492.1, found 492.1. |

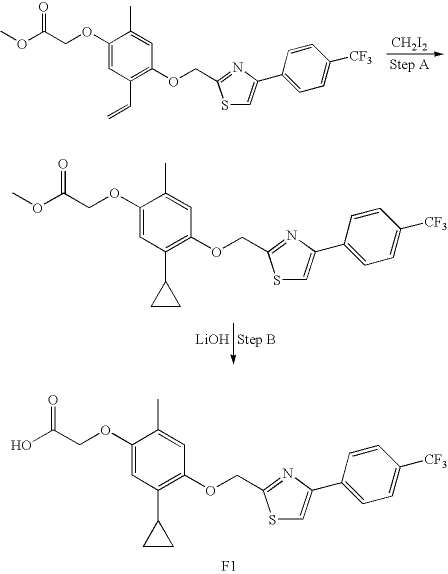

### Example F1

### {5-Cyclopropyl-2-methyl-4-[4-(4-trifluoromethylphenyl)-thiazol-2-ylmethoxy]-phenoxy}-acetic Acid

Step A: Copper powder (19 mg, 0.29 mmol) is slurried in toluene (4 mL). Iodine (0.8 mg, 0.0032 mmol) is added and stirred at rt until the brown color disappeared (˜10 min). Diiodomethane (10.4 μL, 0.129 mmol) and {2-methyl-4-[4-(4-trifluoromethyl-phenyl)-thiazol-2-ylmethoxy]-5-vinyl-phenoxy}-acetic acid methyl ester (30 mg, 0.06 mmol, see example D4) are added and the mixture is heated at reflux for 3 h. Then it is cooled, filtered and concentrated to afford {5-cyclopropyl-2-methyl-4-[4-(4-trifluoromethyl-phenyl)-thiazol-2-ylmethoxy]-phenoxy}-acetic acid methyl ester which is used in Step B without further purification. MS calcd. for C₂₄H₂₃F₃NO₄S (M+H⁺) 478.1, found 478.1.

Step B: {5-Cyclopropyl-2-methyl-4-[4-(4-trifluoromethylphenyl)-thiazol-2-ylmethoxy]-phenoxy}-acetic acid methyl ester is dissolved in THF (1 mL) and a solution of 1 M LiOH in H₂O (0.2 mL) is added. The mixture is stirred for 1 h at rt. Then it is acidified with 1 M HCl (0.25 mL), EtOAc (10 mL) is added and the organic layer is washed with brine. The organic layer is dried (MgSO₄), filtered, concentrated and purified on reverse phase HPLC (H₂O/MeCN gradient) to afford the title compound F1 as a white solid: ¹H-NMR (400 MHz, DMSO-d6) δ=8.39 (s, 1H), 8.19 (d, J=8.0 Hz, 2H), 7.83 (d, J=8.4 Hz, 2H), 6.98 (s, 1H), 6.35 (s, 1H), 5.46 (s, 2H), 4.63 (s, 2H), 2.15 (s, 3H), 0.91 (m, 2H), 0.64 (m, 2H). MS calcd. for C₂₃H₂₁F₃NO₄S (M+H⁺) 464.1, found 464.0.

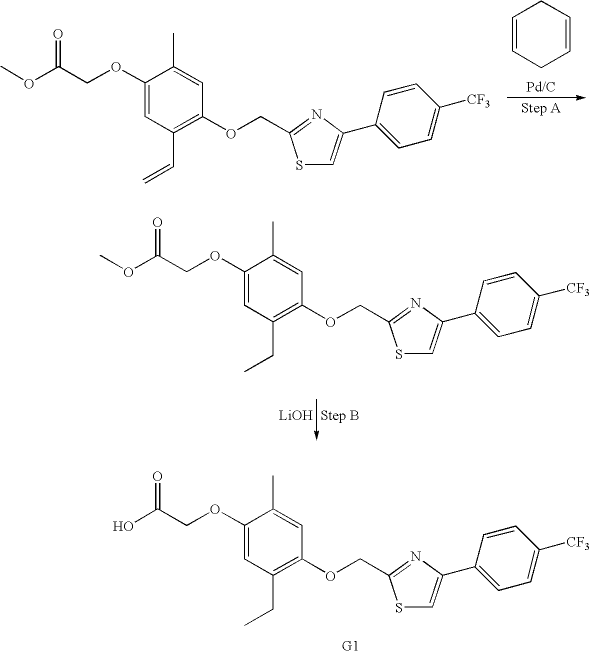

### Example G1

### {5-Ethyl-2-methyl-4-[4-(4-trifluoromethylphenyl)-thiazol-2-ylmethoxy]-phenoxy}-acetic Acid

Step A: {2-Methyl-4-[4-(4-trifluoromethyl-phenyl)-thiazol-2-ylmethoxy]-5-vinyl-phenoxy}-acetic acid methyl ester (40 mg, 0.09 mmol) and 1,4-cyclohexadiene (40 μL, 0.43 mmol) are dissolved in dry MeOH (5 mL). Palladium on charcoal (10 mg) is added and the mixture is stirred at rt for 16 h. The mixture is filtered and concentrated to afford {5-Ethyl-2-methyl-4-[4-(4-trifluoromethyl-phenyl)-thiazol-2-ylmethoxy]-phenoxy}-acetic acid methyl ester which is used in Step B without further purification. MS calcd. for C₂₃H₂₃F₃NO₄S (M+H⁺) 466.1, found 466.1.

Step B: {5-Ethyl-2-methyl-4-[4-(4-trifluoromethyl-phenyl)-thiazol-2-ylmethoxy]-phenoxy}-acetic acid methyl ester is dissolved in THF (1 mL) and a solution of 1 M LiOH in H₂O (0.2 mL) is added. The mixture is stirred for 1 h at rt. Then it is acidified with 1 M HCl (0.25 mL), EtOAc (10 mL) is added and the organic layer is washed with brine. The organic layer is dried (MgSO₄), filtered, concentrated and purified on reverse phase HPLC (H₂O/MeCN gradient) to afford the title compound G1 as a white solid: ¹H-NMR (400 MHz, DMSO-d6) δ=8.38 (s, 1H), 8.19 (d, J=8.0 Hz, 2H), 7.82 (d, J=8.4 Hz, 2H), 6.98 (s, 1H), 6.72 (s, 1H), 5.44 (s, 2H), 4.63 (s, 2H), 2.60 (q, J=7.2 Hz, 2H), 2.17 (s, 3H), 1.16 (t, J=7.2 Hz, 3H). MS calcd. for C₂₂H₂₁F₃NO₄S (M+H⁺) 452.1, found 452.1.

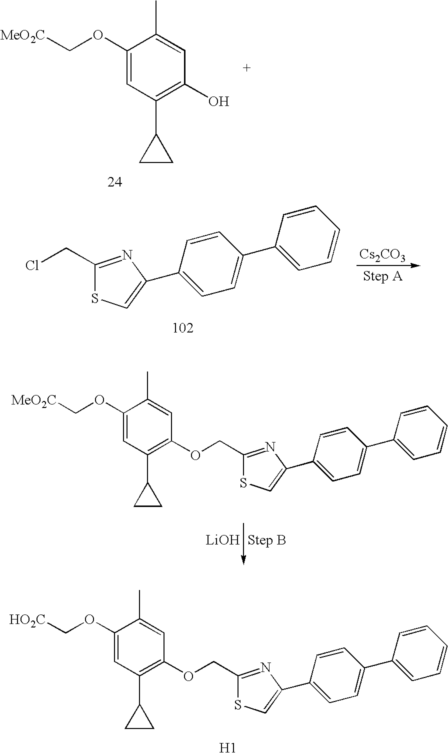

### Example H1

### [4-(4-Biphenyl-4-yl-thiazol-2-ylmethoxy)-5-cyclopropyl-2-methyl-phenoxy]-acetic Acid

Step A: (5-Cyclopropyl-4-hydroxy-2-methyl-phenoxy)-acetic acid methyl ester 24 (40 mg, 0.17 mmol) and 4-Biphenyl-4-yl-2-chloromethyl-thiazole 102 (48 mg, 0.17 mmol) are dissolved in dry acetonitrile (5 mL). Cs₂CO₃ (110 mg, 0.34 mmol) is added and the mixture is heated at reflux for 1 h to afford [4-(4-biphenyl-4-yl-thiazol-2-ylmethoxy)-5-cyclopropyl-2-methyl-phenoxy]-acetic acid methyl ester, which is used in Step B without further purification. MS calcd. for C₂₉H₂₈NO₄S (M+H⁺) 486.2, found 486.1.

Step B: To the solution of [4-(4-Biphenyl-4-yl-thiazol-2-ylmethoxy)-5-cyclopropyl-2-methyl-phenoxy]-acetic acid methyl ester from Step A is added THF (1 mL) and a solution of 1 M LiOH in H₂O (0.2 mL). The mixture is stirred for 1 h at rt. Then it is acidified with 1 M HCl (0.25 mL), EtOAc (10 mL) is added and the organic layer is washed with brine. The organic layer is dried (MgSO₄), filtered, concentrated and purified on reverse phase HPLC (H₂O/MeCN gradient) to afford the title compound H1 as a white solid: ¹H-NMR (400 MHz, CDCl₃) δ=7.98 (d, J=8.4 Hz, 2H), 7.67 (d, J=8.4 Hz, 2H), 7.65 (d, J=7.6 Hz, 2H), 7.54 (s, 1H), 7.46 (t, J=7.2 Hz, 2H), 7.36 (t, J=7.2 Hz, 1H), 6.81 (s, 1H), 6.38 (s, 1H), 5.43 (s, 2H), 4.61 (s, 2H), 2.26 (s, 3H), 2.21 (m, 1H), 0.97 (m, 2H), 0.64 (m, 2H). MS calcd. for C₂₈H₂₆NO₄S (M+H⁺) 472.2, found 472.1.

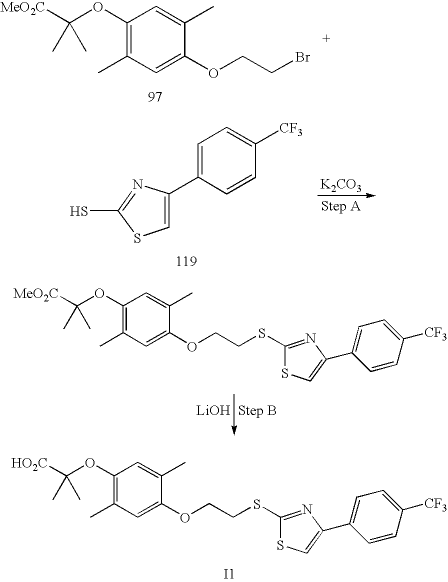

### Example I1

### 2-(2,5-Dimethyl-4-{2-[4-(4-trifluoromethylphenyl)-thiazol-2-ylsulfanyl]-ethoxy}-phenoxy)-2-methyl-propionic Acid

Step A: The mercaptothiazole 119 (0.65 g, 2.49 mmol), the bromide 97 (0.55 g, 1.66 mmol) and K₂CO₃ (0.28 g, 1.99 mmol) are suspended in dry acetonitrile (15 mL) in a sealed tube. The mixture is stirred vigorously and heated to reflux overnight. Then the reaction mixture is cooled to room temperature, filtered and the solvent is removed in vacuo. The remainder is dissolved in EtOAc and washed with water twice, the organic layer is dried (MgSO₄) and concentrated. The remainder is purified by flash chromatography (silica, DCM/MeOH gradient) to afford the 2-(2,5-dimethyl-4-{2-[4-(4-trifluoromethyl-phenyl)-thiazol-2-ylsulfanyl]-ethoxy}-phenoxy)-2-methyl-propionic acid methyl ester as a colourless oil.

Step B: THF (3 mL) and 1 N LiOH (1 mL) are added to the solution derived from step A. The mixture is stirred at 50° C. for 5 h, then acidified with 1 N HCl (˜1.5 mL). The reaction mixture is extracted with DCM (3 mL), the organic layer is separated and concentrated in vacuo. The remainder is taken up in DMSO (1 mL) and purified on reverse phase HPLC (H₂O/MeCN gradient) to afford the title compound I1 as a white solid: ¹H-NMR (600 MHz, CDCl₃) δ=7.96 (d, J=8.0 Hz, 2H), 7.66 (d, J=8.4 Hz, 2H), 7.47 (s, 1H), 6.68 (s, 1H), 6.64 (s, 1H), 4.32 (t, J=6.4 Hz, 2H), 3.71 (t, J=6.4 Hz, 2H), 2.16 (s, 3H), 2.14 (s, 3H), 1.54 (s, 6H). MS calculated for C₂₄H₂₅F₃NO₄S₂ (M+H⁺) 512.1, found 512.0.

By repeating the procedures described in the above examples, using appropriate starting materials, the following compounds of Formula I, as identified in Table 6, are obtained.

TABLE 6

| | | Physical Data |
|---|---|---|
| Compound | Compound | ¹H NMR 400 MHz (DMSO-d₆) |
| Number | Structure | and/or MS (m/z) |
| I2 | 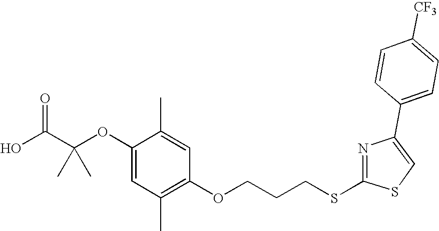 | ¹H-NMR (600 MHz, CDCl₃) δ = 7.96 (d, J = 8.0 Hz, 2 H), 7.66 (d, J = 8.0 Hz, 2 H), 7.46 (s, 1 H), 6.70 (s, 1 H), 6.61 (s, 1 H), 4.08 (t, J = 5.6 Hz, 2 H), 3.50 (t, 3 = 7.2 Hz, 2 H), 2.33 (m, 2 H), 2.18 (s, 3 H), 2.16 (s, 3 H), 1.54 (s, 6 H). MS calculated for C₂₅H₂₇F₃NO₄S₂ (M + H⁺) 526.1, found 526.1. |
| I3 | 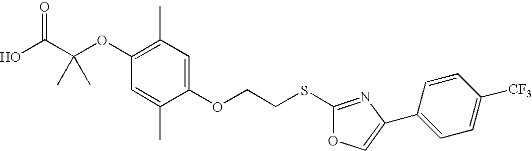 | ¹H-NMR (600 MHz, CDCl₃) δ = 7.92 (d, J = 8.4 Hz, 2 H), 7.64 (d, J = 8.8 Hz, 2 H), 7.01 (s, 1 H), 6.70 (s, 1 H), 6.67 (s, 1 H), 4.87 (t, J = 4.8 Hz, 2 H), 4.33 (t, J = 4.8 Hz, 2 H), 2.21 (s, 3 H), 2.15 (s, 3 H), 1.54 (s, 6 H). MS calculated for C₂₄H₂₅F₃NO₅S (M + H⁺) 496.1, found 496.1. |

### Transcriptional Assay

Transfection assays are used to assess the ability of compounds of the invention to modulate the transcriptional activity of the PPARs. Briefly, expression vectors for chimeric proteins containing the DNA binding domain of yeast GAL4 fused to the ligand-binding domain (LBD) of either PPARδ, PPARα or PPARγ are introduced via transient transfection into mammalian cells, together with a reporter plasmid where the luciferase gene is under the control of a GAL4 binding site. Upon exposure to a PPAR modulator, PPAR transcriptional activity varies, and this can be monitored by changes in luciferase levels. If transfected cells are exposed to a PPAR agonist, PPAR-dependent transcriptional activity increases and luciferase levels rise.

93T human embryonic kidney cells (8×10⁶) are seeded in a 175 cm² flask a day prior to the start of the experiment in 10% FBS, 1% Penicillin/Streptomycin/Fungizome, DMEM Media. The cells are harvested by washing with PBS (30 ml) and then dissociating using trypsin (0.05%; 3 ml). The trypsin is inactivated by the addition of assay media (DMEM, CA-dextran fetal bovine serum (5%). The cells are spun down and resuspended to 170,000 cells/ml. A Transfection mixture of GAL4-PPAR LBD expression plasmid (1 μg), UAS-luciferase reporter plasmid (1 μg), Fugene (3:1 ratio; 6 μL) and serum-free media (200 μL) was prepared and incubated for 15-40 minutes at room temperature. Transfection mixtures are added to the cells to give 0.16M cells/mL, and cells (50 μl/well) are then plated into 384 white, solid-bottom, TC-treated plates. The cells are further incubated at 37° C., 5.0% CO₂ for 5-7 hours. A 12-point series of dilutions (3 fold serial dilutions) are prepared for each test compound in DMSO with a starting compound concentration of 10 μM. Test compound (500 nl) is added to each well of cells in the assay plate and the cells are incubated at 37° C., 5.0% CO₂ for 18-24 hours. The cell lysis/luciferase assay buffer, Bright-Glo™ (25%; 25 μl; Promega), is added to each well. After a further incubation for 5 minutes at room temperature, the luciferase activity is measured.

Raw luminescence values are normalized by dividing them by the value of the DMSO control present on each plate. Normalized data is analyzed and dose-response curves are fitted using Prizm graph fitting program. EC50 is defined as the concentration at which the compound elicits a response that is half way between the maximum and minimum values. Relative efficacy (or percent efficacy) is calculated by comparison of the response elicited by the compound with the maximum value obtained for a reference PPAR modulator.

Compounds of Formula I, in free form or in pharmaceutically acceptable salt form, exhibit valuable pharmacological properties, for example, as indicated by the in vitro tests described in this application. Compounds of the invention preferably have an EC50 for PPARδ and/or PPARα and/or PPARγ, of less than 5 μM, more preferably less than 1 μM, more preferably less than 500 nm, more preferably less than 100 nM. Compounds of the invention preferably have an EC50 for PPARδ that is less than or equal to PPARα which in turn has an EC50 that is at least 10-fold less than PPARγ.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 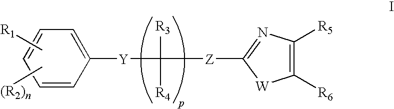
in which
n is selected from 0, 1, 2 and 3;
p is selected from 0, 1, 2 and 3;
Y is selected from O, S, NR₇ₐ and CR₇ₐR_{7b}; wherein R₇ₐ and R_{7b} are independently selected from hydrogen and C₁₋₆alkyl;
Z is selected from CR₈ₐR_{8b} and S; wherein R₈ₐ and R_{8b} are independently selected from hydrogen and C₁₋₆alkyl;
W is selected from O and S;
R₁ is selected from —X₁CR₉R₁₀X₂CO₂R₁₁, —X₁SCR₉R₁₀X₂CO₂R₁₁ and —X₁OCR₉R₁₀X₂CO₂R₁₁; wherein X₁ and X₂ are independently selected from a bond and C₁₋₄alkylene; and R₉ and R₁₀ are independently selected from hydrogen, C₁₋₄alkyl and C₁₋₄alkoxy; or R₉ and R₁₀ together with the carbon atom to which R₉ and R₁₀ are attached form C₃₋₁₂cycloalkyl; and R₁₁ is selected from hydrogen and C₁₋₆alkyl; each
R₂ is independently selected from halo, C₁₋₆alkyl, C₂₋₆alkenyl, C₁₋₄alkoxy, C₁₋₄alkylthio, C₃₋₁₂cycloalkyl, C₃₋₈heterocycloalkyl, C₆₋₁₀aryl and C₅₋₁₀heteroaryl; wherein any aryl, heteroaryl, cycloalkyl or heterocycloalkyl of R₂ is optionally substituted with 1 to 3 radicals independently selected from halo, C₁₋₆alkyl, C₁₋₆alkoxy, C₂₋₆alkenyl, C₁₋₆alkylthio, halo-substituted-C₁₋₆alkyl, halo-substituted-C₁₋₆alkoxy, —C(O)R₁₄ₐ and NR₁₄ₐR_{14b}; wherein R₁₄ₐ and R_{14b} are independently selected from hydrogen and C₁₋₆alkyl;
R₃ and R₄ are independently selected from hydrogen and C₁₋₆alkyl;
R₅ and R₆ are independently selected from hydrogen, C₁₋₆alkyl, C₃₋₁₂cycloalkyl, C₃₋₈heterocycloalkyl, C₆₋₁₀aryl and C₅₋₁₃heteroaryl;
wherein any aryl, heteroaryl, cycloalkyl and heterocycloalkyl of R₅ and R₆ is optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈heterocycloalkyl, C₆₋₁₀aryl, C₅₋₁₃heteroaryl, —XS(O)₀₋₂R¹², —XS(O)₀₋₂XR¹³, —XNR¹²R¹², —XNR¹²S(O)₀₋₂R¹², —XNR¹²C(O)R¹², —XC(O)NR¹²R¹², —XNR¹²C(O)R¹³, —XC(O)NR¹²R¹³, —XC(O)R¹³, —XNR¹²XR¹³ and —XOXR¹³; wherein any aryl, heteroaryl, cycloalkyl or heterocycloalkyl substituent is further optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl and halo-substituted-C₁₋₆alkoxy; wherein X is a bond or C₁₋₄alkylene; R¹² is selected from hydrogen and C₁₋₆alkyl; and R¹³ is selected from C₃₋₁₂cycloalkyl, C₃₋₈heterocycloalkyl, C₆₋₁₀aryl and C₅₋₁₀heteroaryl; wherein any aryl, heteroaryl, cycloalkyl or heterocycloalkyl of R¹³ is optionally substituted with 1 to 3 radicals independently selected from halo, nitro, cyano, C₁₋₆alkyl, C₁₋₆alkoxy, halo-substituted-C₁₋₆alkyl and halo-substituted-C₁₋₆alkoxy; with the proviso that either R₅ or R₆, but not both R₅ and R₆, must be hydrogen or methyl; and the pharmaceutically acceptable salts, hydrates, solvates, isomers and prodrugs thereof.

2. The compound of claim 1 in which:
n is selected from 0, 1, 2 and 3;
p is selected from 0, 1 and 2;
Y is selected from O and S;
Z is selected from CR₈ₐR_{8b} and S; wherein R₈ₐ and R_{8b} are independently selected from hydrogen and C₁₋₆alkyl;
W is selected from O and S;
R₁ is selected from —X₁CR₉R₁₀X₂CO₂R₁₁, —X₁SCR₉R₁₀X₂CO₂R₁₁ and —X₁OCR₉R₁₀X₂CO₂R₁₁; wherein X₁ and X₂ are independently selected from a bond and C₁₋₄alkylene; and R₉ and R₁₀ are independently selected from hydrogen, C₁₋₄alkyl and C₁₋₄alkoxy; or R₉ and R₁₀ together with the carbon atom to which R₉ and R₁₀ are attached form C₃₋₁₂cycloalkyl; and R₁₁ is selected from hydrogen and C₁alkyl; each
R₂ is independently selected from C₁₋₆alkyl, C₂₋₆alkenyl, C₁₋₄alkoxy, C₁₋₄alkylthio, C₃₋₁₂cycloalkyl, C₃₋₈heterocycloalkyl, C₆₋₁₀aryl and C₅₋₁₀heteroaryl; wherein any aryl, heteroaryl, cycloalkyl or heterocycloalkyl of R₂ is optionally substituted with 1 to 3 radicals independently selected from halo, C₁₋₆alkoxy, C₁₋₆alkylthio, halo-substituted-C₁₋₆alkoxy, —C(O)R₁₄ₐ and NR₁₄ₐR_{14b}; wherein R₁₄ₐ and R_{14b} are independently selected from hydrogen and C₁₋₆alkyl;
R₃ and R₄ are independently selected from hydrogen and C₁₋₆alkyl;
R₅ is C₆₋₁₀aryl optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈heterocycloalkyl, C₆₋₁₀aryl, C₅₋₁₃heteroaryl and —XNR¹²R¹²; wherein R¹² is selected from hydrogen and C₁₋₆alkyl; and
R₆ is selected from hydrogen and methyl.

3. The compound of claim 2 in which R₁ is selected from —CH₂CR₅R₆CO₂H, —OCR₅R₆CO₂H, —SCR₅R₆CO₂H, —CR₅R₆CH₂CO₂H and —CR₅R₆CO₂H; wherein R₅ and R₆ are independently selected from hydrogen, methyl, methoxy and ethoxy; or R₅ and R₆ together with the carbon atom to which R₅ and R₆ are attached form cyclopentyl.

4. The compound of claim 3 in which each R₂ is independently selected from methyl, ethyl, cyclopropyl, methoxy, furanyl, phenyl, pyridinyl, thienyl, pyrrolidinyl and benzo[1,3]dioxolyl; wherein said pyridinyl or phenyl of R₂ is optionally substituted with 1 to 3 radicals independently selected from halo, methyl-carbonyl, dimethyl-amino, methoxy, halo-substituted-methoxy, methyl-thio, ethenyl, hexenyl and propyloxy.

5. The compound of claim 1 selected from: (5-Cyclopropyl-2-methyl-4-{3-[4-(4-trifluoromethyl-phenyl)-thiazol-2-yl]-propoxy}-phenoxy)-acetic acid; (3-{3-[4-(4-Trifluoromethyl-phenyl)-thiazol-2-yl]-propoxy}-phenyl)-acetic acid; (2-Methyl-4-{3-[4-(4-trifluoromethyl-phenyl)-thiazol-2-yl]-propoxy}-phenoxy)-acetic acid; 2-Methyl-2-(4-{3-[4-(4-trifluoromethyl-phenyl)-thiazol-2-yl]-propoxy}-phenoxy)-propionic acid; (2-Cyclopropyl-3-{3-[4-(4-trifluoromethyl-phenyl)-thiazol-2-yl]-propoxy}-phenyl)-acetic acid; (4-Cyclopropyl-3-{3-[4-(4-trifluoromethyl-phenyl)-thiazol-2-yl]-propoxy}-phenyl)-acetic acid; (±)-2-Ethoxy-3-(4-{3-[4-(4-trifluoromethyl-phenyl)-thiazol-2-yl]-propoxy}-phenyl)propionic acid; (±)-2-Methoxy-3-(4-{3-[4-(4-trifluoromethyl-phenyl)-thiazol-2-yl]-propoxy}-phenyl)-propionic acid; (3-{3-[4-(4-Trifluoromethyl-phenyl)-oxazol-2-yl]propoxy}-phenyl)-acetic acid; (2-Methyl-4-{3-[4-(4-trifluoromethyl-phenyl)-oxazol-2-yl]-propoxy}-phenoxy)-acetic acid; (5-Cyclopropyl-2-methyl-4-{3-[4-(4-trifluoromethylphenyl)-oxazol-2-yl]-propoxy}-phenoxy)-acetic acid; 2-Methyl-2-(4-{3-[4-(4-trifluoromethyl-phenyl)-oxazol-2-yl]-propoxy}-phenoxy)-propionic acid; (2-Cyclopropyl-3-{3-[4-(4-trifluoromethyl-phenyl)-oxazol-2-yl]-propoxy}-phenyl)-acetic acid; (4-Cyclopropyl-3-{3-[4-(4-trifluoromethyl-phenyl)-oxazol-2-yl]-propoxy}-phenyl)-acetic acid; (3-{2-[4-(4-Trifluoromethyl-phenyl)-oxazol-2-yl]-ethoxy}-phenyl)-acetic acid; (3-{2-[4-(4-Trifluoromethoxy-phenyl)-oxazol-2-yl]-ethoxy}-phenyl)-acetic acid; (2-Cyclopropyl-3-{2-[4-(4-trifluoromethoxy-phenyl)-oxazol-2-yl]-ethoxy}-phenyl)-acetic acid; (4-Cyclopropyl-3-{2-[4-(4-trifluoromethoxy-phenyl)-oxazol-2-yl]-ethoxy}-phenyl)-acetic acid; (2-Cyclopropyl-3-{2-methyl-2-[4-(4-trifluoromethyl-phenyl)-oxazol-2-yl]-propoxy}-phenyl)acetic acid; (4-Cyclopropyl-3-{2-methyl-2-[4-(4-trifluoromethyl-phenyl)-oxazol-2-yl]-propoxy}-phenyl)-acetic acid; 3-(2-Methyl-4-{3-[4-(4-trifluoromethyl-phenyl)-thiazol-2-yl]-propoxy}-phenyl)-propionic acid; 3-(2-Cyclopropyl-5-{3-[4-(4-trifluoromethyl-phenyl)-thiazol-2-yl]-propoxy}-phenyl)-propionic acid; 3-(5-Cyclopropyl-2-methyl-4-{3-[4-(4-trifluoromethyl-phenyl)-thiazol-2-yl]-propoxy}-phenyl)-propionic acid; 3-(4-Cyclopropyl-3-{3-[4-(4-trifluoromethyl-phenyl)-thiazol-2-yl]-propoxy}-phenyl)-propionic acid; 2-Methyl-2-(3-methyl-4-{3-[4-(4-trifluoromethyl-phenyl)-thiazol-2-yl]-propoxy}-phenoxy)-propionic acid; (±)-2-Methyl-3-(4-{3-[4-(4-trifluoromethyl-phenyl)-thiazol-2-yl]-propoxy}-phenyl)propionic acid; (±)-3-(4-{3-[4-(4-Trifluoromethyl-phenyl)-thiazol-2-yl]-propoxy}-phenyl)-butyric acid; 2-Methyl-2-(2-methyl-4-{3-[4-(4-trifluoromethyl-phenyl)-thiazol-2-yl]-propoxy}-phenoxy)-propionic acid; 2-(2,3-Dimethyl-4-{3-[4-(4-trifluoromethyl-phenyl)thiazol-2-yl]-propoxy}-phenoxy)-2-methyl-propionic acid; (±)-2-Ethoxy-3-(2-methyl-4-{3-[4-(4-trifluoromethyl-phenyl)-thiazol-2-yl]-propoxy}-phenyl)-propionic acid; 2-(2,5-Dimethyl-4-{3-[4-(4-trifluoromethyl-phenyl)-thiazol-2-yl]-propylsulfanyl}-phenoxy)-2-methyl-propionic acid; (2,5-Dimethyl-4-{3-[4-(4-trifluoromethyl-phenyl)-thiazol-2-yl]-propoxy}-phenoxy)-acetic acid; (2,5-Dimethyl-4-{3-[4-(4-trifluoromethyl-phenyl)-thiazol-2-yl]-propylsulfanyl}-phenoxy)-acetic acid; 2-(2,5-Dimethyl-4-{3-[4-(4-trifluoromethylphenyl)-thiazol-2-yl]-propoxy}-phenoxy)-2-methyl-propionic acid; (3-Methoxy-4-{3-[4-(4-trifluoromethyl-phenyl)-thiazol-2-yl]-propoxy}-phenyl)-acetic acid; (4-Methoxy-3-{3-[4-(4-trifluoromethyl-phenyl)-thiazol-2-yl]-propoxy}-phenyl)-acetic acid; (±)-3-(2,5-Dimethyl-4{3-[4-(4-trifluoromethyl-phenyl)-thiazol-2-yl]-propoxy}-phenyl)-2-ethoxy-propionic acid; 3-(2,5-Dimethyl-4-{3-[4-(4-trifluoromethyl-phenyl)-thiazol-2-yl]-propoxy}-phenyl)-2,2-dimethyl-propionic acid; 2-(2,5-Dimethyl-4-{3-[4-(4-trifluoromethyl-phenyl)-thiazol-2-yl]-propoxy}-phenylsulfanyl)-2-methyl-propionic acid; (2-Methyl-4-{2-[4-(4-trifluoromethylphenyl)-thiazol-2-yl]-ethoxy}-phenoxy)-acetic acid; (2-Methyl-4-{2-[4-(4-trifluoromethoxyphenyl)-thiazol-2-yl]-ethoxy}-phenoxy)-acetic acid; (2-Methyl-4-{2-[5-methyl-4-(4-trifluoromethyl-phenyl)-thiazol-2-yl]-ethoxy}-phenoxy)-acetic acid; (2-Methyl-4-{2-[4-(3-trifluoromethyl-phenyl)-thiazol-2-yl]-ethoxy}-phenoxy)-acetic acid; (4-{2-[4-(4-Methoxyphenyl)-thiazol-2-yl]-ethoxy}-2-methyl-phenoxy)-acetic acid; {2-Methyl-4-[2-(4-naphthalen-2-yl-thiazol-2-yl)-ethoxy]-phenoxy}-acetic acid; (2-Methyl-4-{3-[4-(4-trifluoromethyl-phenyl)-thiazol-2-yl]-propoxy}-phenoxy)-acetic acid; (2-Methyl-4-{3-[4-(4-trifluoromethoxy-phenyl)-thiazol-2-yl]-propoxy}-phenoxy)-acetic acid; (2-Methyl-4-{3-[5-methyl-4-(4-trifluoromethyl-phenyl)-thiazol-2-yl]-propoxy}-phenoxy)-acetic acid; (2-Methyl-4-{3-[4-(3-trifluoromethyl-phenyl)-thiazol-2-yl]-propoxy}-phenoxy)-acetic acid; (4-{3-[4-(4-Methoxy-phenyl)-thiazol-2-yl]-propoxy}-2-methyl-phenoxy)-acetic acid; {2-Methyl-4-[3-(4-naphthalen-2-yl-thiazol-2-yl)-propoxy]-phenoxy}-acetic acid; (2-Methyl-4-{4-[4-(4-trifluoromethyl-phenyl)-thiazol-2-yl]-butoxy}-phenoxy)-acetic acid; (2-Methyl-4-{4-[4-(4-trifluoromethoxy-phenyl)-thiazol-2-yl]-butoxy}-phenoxy)-acetic acid; (2-Methyl-4-{4-[5-methyl-4-(4-trifluoromethyl-phenyl)-thiazol-2-yl]-butoxy}-phenoxy)-acetic acid; (2-Methyl-4-{4-[4-(3-trifluoromethyl-phenyl)-thiazol-2-yl]-butoxy}-phenoxy)-acetic acid; (4-{4-[4-(4-Methoxy-phenyl)-thiazol-2-yl]-butoxy}-2-methyl-phenoxy)-acetic acid; {2-Methyl-4-[4-(4-naphthalen-2-yl-thiazol-2-yl)-butoxy]-phenoxy}-acetic acid; (5-Cyclopropyl-2-methyl-4-{2-[4-(4-trifluoromethyl-phenyl)-thiazol-2-yl]-ethoxy}-phenoxy)-acetic acid; {5-Cyclopropyl-2-methyl-4-[2-(4-naphthalen-2-yl-thiazol-2-yl)-ethoxy]-phenoxy}-acetic acid; (5-Cyclopropyl-2-methyl-4-{2-[5-methyl-4-(4-trifluoromethyl-phenyl)-thiazol-2-yl]-ethoxy}-phenoxy)-acetic acid; (3-{2-[4-(4-Trifluoromethyl-phenyl)-thiazol-2-yl]-ethoxy}-phenyl)-acetic acid; {3-[2-(4-Naphthalen-2-yl-thiazol-2-yl)-ethoxy]-phenyl}-acetic acid; (3-{2-[5-Methyl-4-(4-trifluoromethyl-phenyl)-thiazol-2-yl]-ethoxy}-phenyl)-acetic acid; (2-Cyclopropyl-3-{2-[4-(4-trifluoromethyl-phenyl)-thiazol-2-yl]-ethoxy}-phenyl)-acetic acid; {2-Cyclopropyl-3-[2-(4-naphthalen-2-yl-thiazol-2-yl)-ethoxy]-phenyl}-acetic acid; (2-Cyclopropyl-3-{2-[5-methyl-4-(4-trifluoromethyl-phenyl)-thiazol-2-yl]-ethoxy}-phenyl)-acetic acid; (4-Cyclopropyl-3-{2-[4-(4-trifluoromethyl-phenyl)-thiazol-2-yl]-ethoxy}-phenyl)-acetic acid; {4-Cyclopropyl-3-[2-(4-naphthalen-2-yl-thiazol-2-yl)-ethoxy]-phenyl}-acetic acid; (4-Cyclopropyl-3-{2-[5-methyl-4-(4-trifluoromethyl-phenyl)-thiazol-2-yl]-ethoxy}-phenyl)-acetic acid; 2-Methyl-2-(4-{2-[4-(4-trifluoromethyl-phenyl)-thiazol-2-yl]-ethoxy}-phenoxy)-propionic acid; 2-Methyl-2-{4-[2-(4-naphthalen-2-yl-thiazol-2-yl)ethoxy]-phenoxy}-propionic acid; 2-Methyl-2-(4-{2-[5-methyl-4-(4-trifluoromethylphenyl)-thiazol-2-yl]-ethoxy}-phenoxy)-propionic acid; {4′-Methoxy-4-methyl-6-[4-(4-trifluoromethyl-phenyl)-thiazol-2-ylmethoxy]-biphenyl-3-yloxy}-acetic acid; {4-Methyl-4′-trifluoromethoxy-6-[4-(4-trifluoromethyl-phenyl)-thiazol-2-ylmethoxy]-biphenyl-3-yloxy}-acetic acid; {4-Methyl-4′-methylsulfanyl-6-[4-(4-trifluoromethyl-phenyl)-thiazol-2-ylmethoxy]-biphenyl-3-yloxy}-acetic acid; {2-Methyl-4-[4-(4-trifluoromethyl-phenyl)thiazol-2-ylmethoxy]-5-vinyl-phenoxy}-acetic acid; {5-Hex-1-enyl-2-methyl-4-[4-(4-trifluoromethyl-phenyl)-thiazol-2-ylmethoxy]-phenoxy}-acetic acid; {5-Furan-3-yl-2-methyl-4-[4-(4-trifluoromethyl-phenyl)-thiazol-2-ylmethoxy]-phenoxy}-acetic acid; {5-(6-Methoxy-pyridin-3-yl)-2-methyl-4-[4-(4-trifluoromethyl-phenyl)-thiazol-2-ylmethoxy]-phenoxy}-acetic acid; {4-Methyl-4′-propoxy-6-[4-(4-trifluoromethyl-phenyl)-thiazol-2-ylmethoxy]-biphenyl-3-yloxy}-acetic acid; {3′-Chloro-4′-methoxy-4-methyl-6-[4-(4-trifluoromethyl-phenyl)-thiazol-2-ylmethoxy]-biphenyl-3-yloxy}-acetic acid; {5-Benzo[1,3]dioxol-5-yl-2-methyl-4-[4-(4-trifluoromethyl-phenyl)-thiazol-2-ylmethoxy]-phenoxy}-acetic acid; {4′-Acetyl-4-methyl-6-[4-(4-trifluoromethyl-phenyl)-thiazol-2-ylmethoxy]-biphenyl-3-yloxy}-acetic acid; {4′-Dimethylamino-4-methyl-6-[4-(4-trifluoromethyl-phenyl)-thiazol-2-ylmethoxy]-biphenyl-3-yloxy}-acetic acid; {2-Methyl-5-thiophen-2-yl-4-[4-(4-trifluoromethyl-phenyl)-thiazol-2-ylmethoxy]-phenoxy}-acetic acid; {4-Methyl-4′-methylsulfanyl-6-[4-(4-trifluoromethoxy-phenyl)-thiazol-2-ylmethoxy]-biphenyl-3-yloxy}-acetic acid; {3′-Methoxy-4-methyl-6-[4-(4-trifluoromethoxy-phenyl)thiazol-2-ylmethoxy]-biphenyl-3-yloxy}-acetic acid; {2-Methyl-5-pyrrolidin-1-yl-4-[4-(4-trifluoromethyl-phenyl)-thiazol-2-ylmethoxy]-phenoxy}-acetic acid; (±)-(4′-Methoxy-4-methyl-6-{1-[4-(4-trifluoromethyl-phenyl)-thiazol-2-yl]-ethoxy}-biphenyl-3-yloxy)-acetic acid; (4′-Methoxy-4-methyl-6-{2-[4-(4-trifluoromethyl-phenyl)-thiazol-2-yl]-ethoxy}-biphenyl-3-yloxy)-acetic acid; (2-Methyl-4-{2-[4-(4-trifluoromethyl-phenyl)-thiazol-2-yl]-ethoxy}-5-vinyl-phenoxy)-acetic acid; {4′-Methoxy-4-methyl-6-[4-(4-nitro-phenyl)-thiazol-2-ylmethoxy]-biphenyl-3-yloxy}-acetic acid; {4′-Methoxy-6-[4-(4-methoxy-phenyl)-thiazol-2-ylmethoxy]-4-methyl-biphenyl-3-yloxy}-acetic acid; [4′-Methoxy-4-methyl-6-(4-p-tolyl-thiazol-2-ylmethoxy)-biphenyl-3-yloxy]-acetic acid; {6-[4-(4-Chloro-phenyl)-thiazol-2-ylmethoxy]-4′-methoxy-4-methyl-biphenyl-3-yloxy}-acetic acid; {6-[4-(4-Diethylaminophenyl)-thiazol-2-ylmethoxy]-4′-methoxy-4-methyl-biphenyl-3-yloxy}-acetic acid; {6-[4-(4-Cyano-phenyl)-thiazol-2-ylmethoxy]-4′-methoxy-4-methyl-biphenyl-3-yloxy}-acetic acid; [6-(4-Biphenyl-4-yl-thiazol-2-ylmethoxy)-4′-methoxy-4-methyl-biphenyl-3-yloxy]-acetic acid; [4′-Methoxy-4-methyl-6-(4-naphthalen-2-yl-thiazol-2-ylmethoxy)-biphenyl-3-yloxy]-acetic acid; {4′-Methoxy-6-[4-(3-methoxy-phenyl)-thiazol-2-ylmethoxy]-4-methyl-biphenyl-3-yloxy}-acetic acid; {5-Cyclopropyl-2-methyl-4-[4-(4-trifluoromethyl-phenyl)-thiazol-2-ylmethoxy]-phenoxy}-acetic acid; {5-Ethyl-2-methyl-4-[4-(4-trifluoromethyl-phenyl)thiazol-2-ylmethoxy]-phenoxy}-acetic acid; [4-(4-Biphenyl-4-yl-thiazol-2-ylmethoxy)-5-cyclopropyl-2-methyl-phenoxy]-acetic acid; 2-(2,5-Dimethyl-4-{2-[4-(4-trifluoromethylphenyl)-thiazol-2-ylsulfanyl]-ethoxy}-phenoxy)-2-methyl-propionic acid; and 2-(2,5-Dimethyl-4-{3-[4-(4-trifluoromethyl-phenyl)-thiazol-2-ylsulfanyl]-propoxy}-phenoxy)-2-methyl-propionic acid.

6. A method for treating a disease or disorder in an animal in which modulation of PPAR activity can prevent, inhibit or ameliorate the pathology and/or symptomology of the disease, which method comprises administering to the animal a therapeutically effective amount of a compound of claim 1.

7. The method of claim 6 in which the PPAR activity is at least one PPAR selected from PPARα, PPARδ and PPARγ.

8. The method of claim 7 in which the PPAR activity is both PPARα and PPARδ.

9. The method of claim 6 in which the disease or disorder is selected from the treatment of prophylaxis, dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, cachexia, inflammation, arthritis, cancer, anorexia, anorexia nervosa, bulimia, Alzheimer's disease, skin disorders, respiratory diseases, ophthalmic disorders, irritable bowel diseases, ulcerative colitis, Crohn's disease, type-1 diabetes, type-2 diabetes and Syndrome X.

10. The method of claim 6 in which the disease or disorder is selected from HIV wasting syndrome, long term critical illness, decreased muscle mass and/or muscle strength, decreased lean body mass, maintenance of muscle strength and function in the elderly, diminished muscle endurance and muscle function, and frailty in the elderly.

11. The use of a compound according to any of claims 1 to 5 in the manufacture of a medicament for treating a disease in an animal in which PPAR activity contributes to the pathology and/or symptomology of the disease.

12. The use of claim 11 in which the PPAR activity is at least one PPAR selected from PPARα, PPARβ and PPARγ.

13. The use of claim 12 in which the PPAR activity is both PPARα and PPARδ.

14. A pharmaceutical composition comprising a therapeutically effective amount of a compound of any of claim 1 to 5 in combination with one or more pharmaceutically acceptable excipients.

15. A pharmaceutical combination, especially a pharmaceutical composition, comprising: 1) a compound of any of claims 1 to 5 or a pharmaceutical acceptable salt thereof; and 2) at least one active ingredient selected from:
a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; dipeptidyl peptidase IV inhibitors such as DPP728, vildagliptin, MK-0431, saxagliptin, GSK23A; an AGE breaker; a thiazolidone derivative (glitazone) such as pioglitazone, rosiglitazone, or (R)-1-{4-[5-methyl-2-(4-trifluoromethylphenyl)-oxazol-4-ylmethoxy]-benzenesulfonyl}-2,3-dihydro-1H-indole-2-carboxylic acid, a non-glitazone type PPARγ agonist e.g. GI-262570;
b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) an anti-obesity agent or appetite regulating agent such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine or cannabinoid receptor antagonists;
d) anti-hypertensive agents, e.g., loop diuretics such as ethacrynic acid, furosemide and torsemide; diuretics such as thiazide derivatives, chlorithiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors;
e) a HDL increasing compound;
f) a cholesterol absorption modulator such as Zetia® and KT6-971;
g) Apo-A1 analogues and mimetics;
h) thrombin inhibitors such as Ximelagatran;
i) aldosterone inhibitors such as anastrazole, fadrazole, eplerenone;
j) Inhibitors of platelet aggregation such as aspirin, clopidogrel bisulfate;
k) estrogen, testosterone, a selective estrogen receptor modulator, a selective androgen receptor modulator;
l) a chemotherapeutic agent such as aromatase inhibitors e.g. femara, antiestrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib or 4-Methyl-N-[3-(4-methyl-imidazol-1-yl)-5-trifluoromethyl-phenyl]-3-(4-pyridin-3-yl-pyrimidin-2-ylamino)-benzamide; and
m) an agent interacting with a 5-HT₃ receptor and/or an agent interacting with 5-HT₄ receptor such as tegaserod, tegaserod hydrogen maleate, cisapride, cilansetron;
or, in each case a pharmaceutically acceptable salt thereof; and optionally a pharmaceutically acceptable carrier.

16. A pharmaceutical composition according to claim 14 or a combination according to claim 15, for the treatment or prevention of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, IBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.

17. A compound according to any of claims 1 to 5, or a pharmaceutical composition according to claim 10 or a combination according to claim 11, for use as a medicament.

18. Use of a compound according to any of claims 1 to 5, or a pharmaceutical composition according to claim 14 or a combination according to claim 15, for the manufacture of a medicament for the treatment or prevention of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, IBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.
